(19) United States
(12) Patent Application Publication
(10) Pub. No.: US 2026/0110689 A1
(43) Pub. Date: Apr. 23, 2026

(54) METHODS AND COMPOSITIONS RELATED TO MODIFIED FLUOROPHORES

(71) Applicant: Board of Regents, The University of Texas System, Austin, TX (US)

(72) Inventors: Emilia Gomez, San Antonio, TX (US); Syed Muhammad Usama, San Antonio, TX (US)

(21) Appl. No.: 19/364,849

(22) Filed: Oct. 21, 2025

Related U.S. Application Data
(60) Provisional application No. 63/709,546, filed on Oct. 21, 2024.

(51) Int. Cl. G01N 33/53; C09B 23/01; C09B 23/08; C09K 11/06; G01N 33/58

(52) U.S. Cl. CPC G01N 33/582; C09B 23/0008; C09B 23/005; C09B 23/083; C09K 11/06; G01N 33/5306; C09K 2211/1007; C09K 2211/1018

(57) ABSTRACT

The present invention provides a novel one-step synthesis method for producing cyanine-based fluorophores with modified polymethine chains to prevent aggregation, enhancing their utility in biomedical research. These fluorophores, such as unsymmetrical heptamethine cyanines like SAT-IR-746, exhibit improved sensitivity, photostability, and brightness, making them ideal for applications such as live-cell imaging, biomolecule labeling, and near-infrared fluorescence microscopy. The synthesis process involves the preparation of a pyridinium benzoxazole (PyBox) intermediate from a substituted pyridine derivative and 2-chlorobenzoxazole, followed by condensation with sulfonated and substituted indolenines to form asymmetrical cyanine dyes, achieving high yields (e.g., 55-82% for SAT-IR series compounds). By addressing the aggregation issue common in traditional cyanine dyes through β-position substitution on the polymethine chain, this invention enables more accurate and reliable labeling and tracking of biomolecules without quenching, facilitating advanced imaging techniques and deeper insights into biological processes.

### PRIORITY PARAGRAPH

This application claims priority to U.S. Provisional Application 63/709,546 filed Oct. 21, 2024 which is incorporated herein by reference in its entirety.

### STATEMENT REGARDING FEDERALLY FUNDED RESEARCH

None.

### BACKGROUND

Fluorescent dyes, particularly polymethine-based fluorophores such as cyanines and oxonols, are essential tools in biomedical research for applications including cellular imaging, membrane potential measurement, and biomolecule labeling. However, these dyes often aggregate in aqueous or biological environments, leading to self-quenching of fluorescence, reduced photostability, and interference with target binding. Existing mitigation strategies, such as sulfonation or PEGylation, often compromise brightness, synthetic yield, or accessibility due to increased production complexity.

There remains a need for simplified, efficient methods to design non-aggregating fluorophores that retain the desirable properties of oxonol and cyanine dyes for biomedical applications.

### SUMMARY

The present invention provides solutions to the problems described above through methods and compositions providing a novel one-step modification of the polymethine chain in oxonol or cyanine scaffolds resulting in photostable, aggregation-resistant dyes with emission in the visible to near-infrared range. Photostable polymethine fluorophores enable applications in biomedical imaging, membrane potential sensing, and biomolecule labeling without interference from aggregation-induced quenching.

Certain embodiments are directed to methods for synthesizing a non-aggregating unsymmetrical heptamethine cyanine fluorophore. The methods include (a) preparing a pyridinium benzoxazole (PyBox) intermediate by reacting a pyridine derivative substituted at the 3- or 4-position (e.g., 3-phenylpyridine) with 2-chlorobenzoxazole in acetonitrile at 70-100° C. for 12-18 hours precipitating the product in diethyl ether, centrifuging, and drying under vacuum; and (b) synthesizing the cyanine dye by reacting the PyBox intermediate with a sulfonated indolenine and a substituted indolenine in ethanol with sodium acetate at 80° C. for 15-60 minutes. In certain aspects, optionally adding triethylamine. Purifying the product using normal or reverse phase chromatography with a dichloromethane/methanol or acetonitrile/water gradient. In certain aspects the unsymmetrical heptamethine cyanine is substituted at the β-position of the polymethine chain with a phenyl group to disrupt molecular planarity and prevent aggregation, e.g., SAT-IR-746.

Certain embodiments are directed to a non-aggregating unsymmetrical heptamethine cyanine fluorophore produced by the methods described herein. The unsymmetrical heptamethine having a delocalized π-electron system with a modified polymethine chain (n=3) and β-substitution, characterized by absorption/emission in the near-infrared range (˜746-758 nm), extinction coefficient >10\hat{ }5 M⁻¹ cm⁻¹, quantum yield 0.1-0.2, and resistance to aggregation in aqueous environments. In certain aspects the fluorophore can further include a bioconjugation handle selected from the group consisting of NHS ester, maleimide, carboxylic acid, or thiol-reactive group, suitable for labeling biomolecules. In certain aspects the fluorophore is configured for use in live-cell imaging, exhibiting stability in physiological conditions (e.g., >80% retention after 18 hours in 10% FBS or pH 5-7.2) and tissue penetration greater than 4 mm.

Certain embodiments are directed to a composition comprising the non-aggregating unsymmetrical heptamethine cyanine fluorophore and a biologically compatible carrier for use in near-infrared fluorescence microscopy, flow cytometry, or in vivo imaging.

Certain embodiments are directed to methods for labeling a biomolecule, comprising (a) providing the non-aggregating unsymmetrical heptamethine cyanine fluorophore and (b) contacting the biomolecule with the fluorophore under conditions sufficient to form a labeled biomolecule. In certain aspects the labeling achieves a DOL of 1-5 without aggregation-induced quenching. In certain aspects the biomolecule is selected from the group consisting of an antibody, peptide, nucleic acid, or cellular membrane component, and the method is for targeted imaging or multiplexing multiple targets.

Other embodiments are directed to kits for biomedical imaging, comprising a non-aggregating unsymmetrical heptamethine cyanine fluorophore. The Unsymmetric Polymethine Fluorophore Kit offers a comprehensive collection of asymmetric cyanine-based dyes designed for advanced biomolecular labeling and fluorescence imaging applications. These fluorophores, characterized by their non-identical end groups linked via a polymethine chain, provide tunable absorption and emission spectra extending into the near-infrared (NIR) range, enabling deeper tissue penetration and reduced autofluorescence in biological samples. Ideal for researchers in chemistry, biology, and diagnostics, the kit includes reagents for simple one-step conjugation to proteins, peptides, or other amine-containing molecules, with enhanced photostability and minimal dye-dye quenching to support high-resolution imaging techniques like NIR-II fluorescence. This innovative tool empowers precise visualization in live-cell studies, medical diagnostics, and nanotechnology, bridging classic organic synthesis with cutting-edge fluorogenic strategies. The kit can also contain specialized buffers, purification columns, and detailed protocols to ensure efficient and reproducible conjugation. This innovative tool empowers precise visualization in live-cell studies, medical diagnostics, and nanotechnology, bridging classic organic synthesis with cutting-edge fluorogenic strategies.

Any embodiment discussed with respect to one aspect of the invention applies to other aspects of the invention as well and vice versa.

The use of the word “a” or “an” when used in conjunction with the term “comprising” in the claims and/or the specification may mean “one,” but it is also consistent with the meaning of “one or more,” “at least one,” and “one or more than one.”

The term “about” is used to indicate that a value includes the standard deviation of error for the device or method being employed to determine the value.

The term “or” in the claims is used to mean “and/or” unless explicitly indicated to refer to alternatives only or the alternatives are mutually exclusive.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended.

The transitional phrases “consists of” and “consisting of” exclude any element, step, or component not specified. When the phrase “consists of” or “consisting of” appears in a clause of the body of a claim, rather than immediately following the preamble, the phrase “consists of” or “consisting of” limits only the elements (or components or steps) set forth in that clause.

The transitional phrases “consists essentially of” and “consisting essentially of” are used to define a composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

The detailed description and the specific examples while indicating specific embodiments of the invention are given by way of illustration only, since various changes and modifications within the spirit and scope of the invention will become apparent to those skilled in the art from this detailed description. The invention is not limited to the embodiments described herein.

Definitions—For the purposes of this patent application, the following terms shall have the meanings set forth below unless otherwise indicated by the context.

Polymethine Dye: A class of synthetic organic compounds characterized by a conjugated chain of methine groups (—CH═) typically flanked by electron-donating and electron-accepting groups.

Cyanine dyes are characterized by a conjugated polymethine chain with two nitrogen-containing heterocyclic end groups. Their general chemical structure can be represented as: R₂N—(CH═CH)ₙ—CH═N⁺R₂ where R represents alkyl or aryl groups attached to the nitrogen atoms, N atoms are part of heterocyclic systems (e.g., indole, benzothiazole, or quinoline derivatives), n is the number of vinyl (—CH═CH—) units in the polymethine chain, which determines the dye's conjugation length and spectral properties (e.g., n=1 for trimethine, n=2 for pentamethine, n=3 for heptamethine cyanines, etc.). The positive charge is typically delocalized across the conjugated system, and a counterion (e.g., Cl⁻, I⁻) balances the charge. The heterocyclic end groups are often identical (symmetric cyanines) but can differ (asymmetric cyanines). Common heterocycles include indole, benzoxazole, benzothiazole, or quinoline. The length of the polymethine chain and the nature of the heterocycles influence the absorption and emission wavelengths, making cyanines versatile for applications like fluorescence imaging.

Near-Infrared (NIR) Region: The portion of the electromagnetic spectrum ranging from approximately 750 nm to 900 nm, valued in biomedical imaging for its low background autofluorescence and ability to penetrate deep tissue.

Pyridinium Benzoxazole: A heterocyclic compound comprising a pyridine ring fused or conjugated with a benzoxazole moiety, used as a starting material or intermediate in the synthesis of cyanine dyes, capable of undergoing ring-opening reactions to facilitate polymethine chain modification.

Terminal group derivative: Terminal group derivative is a modifiable end group or capping moiety attached to the conjugated polymethine chain in cyanine dyes. These are typically nitrogen-containing heterocyclic systems (or their derivatives) that flank the polymethine bridge, influencing the dye's optical properties (e.g., absorption and emission wavelengths above 850 nm), stability, solubility, and suitability for bio-conjugation.

Indole derivative: Indole derivative is a specific example or subtype of a terminal group derivative, where the end group is based on an indole heterocycle (a fused benzene-pyrrole ring system containing nitrogen). Indole derivatives are commonly used in cyanine synthesis due to their ability to extend conjugation and shift emission into the NIR range, making them ideal for deep-tissue imaging.

Ring-Opening Reaction: A chemical reaction involving the cleavage of a cyclic structure, specifically the pyridinium benzoxazole, through nucleophilic addition, enabling the formation of an extended conjugated system in the synthesis of cyanine dyes.

Nucleophilic Addition: A chemical reaction wherein a nucleophile, such as phenylacetaldehyde, donates an electron pair to an electrophilic site on the pyridinium benzoxazole, resulting in the formation of a new chemical bond and the opening of the cyclic structure.

Fluorophore: A chemical compound that can absorb light at a specific wavelength and emit light at a longer wavelength, can refer to cyanine dyes with fluorescence properties suitable for imaging applications.

LC/MS: Liquid chromatography/mass spectrometry, an analytical technique used to monitor the progress of the synthesis reactions by separating and identifying reaction products based on their mass-to-charge ratio.

Bioconjugation: The process of chemically linking a fluorophore, such as a cyanine dye, to a biomolecule (e.g., antibody, peptide, or nucleic acid) to enable targeted imaging or detection in biological systems.

Aggregation: Refers to the clustering or self-association of dye molecules in aqueous or biological environments. This phenomenon, often observed in dyes like indocyanine green (ICG), leads to the formation of non-fluorescent or weakly fluorescent complexes, causing fluorescence quenching, reduced photostability, and interference with biomolecule interactions.

Cyclized: Refers to a chemical process or structural state in which a molecule forms a cyclic (ring-like) structure through the bonding of atoms within the same molecule. This often involves a reversible transformation between different molecular forms, such as an open-chain and a closed-ring configuration, influenced by environmental factors like solvent or pH. Cyclization can alter the molecule's properties, such as its reactivity, stability, or optical characteristics.

Cyclizing: Refers to the process of forming a cyclic (ring-like) structure within a molecule by creating a bond between two atoms, typically within the same molecular chain or framework. This process can be reversible or irreversible and is often influenced by environmental conditions such as solvent, pH, or temperature.

Spirocyclized: Refers to a specific type of cyclization where a molecule contains a spirocyclic structure, characterized by two or more rings sharing a single common atom, known as the spiro atom. This unique arrangement creates a rigid, three-dimensional framework, often enhancing the molecule's stability or influencing its chemical and physical properties, such as fluorescence or solubility.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1A-1B (A) Representative strategies to prevent aggregation of heptamethine cyanines (Cy7) by installing charged (sulfonate, quaternary ammonium) or PEGylated groups on the indolenine rings, at the meso-position, or around the cyclohexyl moiety. (B) introduce Unsymmetrical heptamethine cyanines are introduced as a general approach to prevent aggregation by disrupting molecular planarity and self-association. The meso-position is defined as the α-carbon (Cα); the adjacent position is referred as the β-carbon (Cβ), followed sequentially by the γ-, δ-, and further positions along the polymethine chain.

FIG. 2 Overview of the synthesis of α-(symmetrical) and β-(unsymmetrical) substituted SAT-IR fluorophores. The synthetic route begins with the preparation of pyridinium intermediates (1A-B), featuring either 3-(A) or 4-(B) phenyl substitution (highlighted in pink). These intermediates are then coupled with 2-chlorobenzoxazole and a sulfonated indolenine (D) to afford the SAT-IR-758 (α-Ph) and SAT-IR-746 (β-Ph) fluorophores.

FIG. 3A-3H Absorbance spectra of SAT-IR-758 (top row) and SAT-IR-746 (bottom row) in methanol (a and e), Mili-Q H₂O (b and f), 0.9% Brine (c and g) and 10% FBS+Mili-Q H₂O (d and h) and at increasing concentrations (0.5 μM-20 μM).

FIG. 4A-4H Effect of different salt concentration (154 mM) absorbance in SAT-IR-758 (20 μM) in (A) sodium salts, (B) potassium salts, (C) other common salts and (D) effect of different range of calcium chloride salt concentration (7.81 mM to 500 mM) and SAT-IR-746 (20 μM) in (E) sodium salts, (F) potassium salts, (G) other common salts and (H) effect of different range of calcium chloride salt concentration (7.81 mM to 500 mM)). Ionic strengths: Na2SO4 (37.5 mM), NaI (150 mM), CH3CO₂Na (150 mM), KCl (150 mM), KI (150 mM), KF (150 mM), CaCl₂) (60 mM), NH4Cl (150 mM), TBABr (teratbutylammonium bromide; 150 mM).

FIG. 5A-5D. Absorbance (0.5-20 μM) in MQ water of a series of indolenine substituted unsymmetric heptamethine fluorophores that contains (A) PEGylated (SAT-IR-745), (B) quaternary amine (SAT-IR-741) and (C) zwitterionic (SAT-IR-747) derivative.

FIG. 6A-6D. Absorbance (0.5-20 μM) in MQ water of a series of polymethine substituted unsymmetric heptamethine fluorophores that contains (A) methyl (aliphatic substituent; SAT-IR-743), (B) methoxy (electron donating group; SAT-IR-761) and (C) carboxy (electron withdrawing group; SAT-IR-748) derivative.

FIG. 7A-7F. (A) Synthesis of carboxylic acid-functionalized SAT-IR-746 and SAT-IR-758 fluorophores derived from 3- and 4-pyridine benzoic acid starting materials. The carboxylic acid groups were subsequently converted to NHS esters and conjugation to lysine residues on IgG antibodies. Degree of labeling (DOL) measured at various dye-to-antibody ratios (1-5) for (B) SAT-IR-746, (C) SAT-IR-758, and (D) IRDye 800CW. (E) ratio of absorbance at maximum wavelength to absorbance at shoulder peak against different DOL of IgG-fluorophore conjugates (10 ug in PBS pH 7.2) and (F) fluorescence intensity of IgG-fluorophore conjugates (10 ug in PBS pH 7.2) plotted against DOL.

FIG. 8A-8B. Quantification of H3S10P immunolabeling of mitotic chromatin. (A) Representative single confocal images of HeLa prometaphase nuclei labeled with anti-H3S10P primary antibody followed by secondary antibodies conjugated to NIR fluorophores of interest, as labelled in the figure. All secondary antibodies were used at 3 μg/mL. The top panel shows the NIR image; the bottom panel shows the merge image with the Hoechst signal in blue, H3S10P in yellow. (B) Quantification of H3S10P mean fluorescence intensity (MFI); n>=20 nuclei for each NIR fluorophore of interest.

FIG. 9A-9F. Ex vivo imaging of tissue penetration profiling for SAT-IR-746.(A) Schematic of the experimental setup for tissue penetration imaging using porcine skin and muscle. (B) Fluorescence images of SAT-IR-746 dye at different concentrations (1. 1000 μg/mL, 2. 500 μg/mL, 3. 200 μg/mL, 4. 100 μg/mL, 5. 50 μg/mL, 6. 10 μg/mL, 7. PBS 1×) at the top and SATIR-746 phantoms in different concentrations at bottom. Quantification of fluorescence radiant efficiency as a function of dye concentration in vials (C) and phantoms (D) SAT-IR-746 phantoms covered with increasing thicknesses (2-8 mm) of porcine muscle (E) and (0-4 mm) of porcine skin (F) captured using the IVIS imaging system with an excitation wavelength of 740 nm and emission collection at 790 nm along corresponding quantification of fluorescence radiant efficiency as a function of tissue thickness.

FIG. 10 Non-aggregating symmetric heptamethine structures in literature: IRDye800CW (Li—COR), ZW800, QuatCy and s775z.

FIG. 11. Solvent stability of SAT-IR-746 for 18 h in 3 different aqueous solvents (pH 5.0, 10% FBS+Mili-Q Water, and PBS pH 7.2). The experiment was repeated thrice. Mean and standard deviation of mean is plotted.

FIG. 12A-12B. Hemocompatibility studies of SAT IR-746 dye with porcine blood. (A) White light image of RBC pellets from each incubated sample: (1) 100 μg/mL, (2) 50 μg/mL, (3) 10 μg/mL, (4) 5 μg/mL, (5) 1 μg/mL, (6) PBS (1×), and (7) DI water. (B) Relative hemolysis percentages of all SAT-IR-746 treatment and control groups.

FIG. 13A-13D. Photophysical properties of fluorophores SAT-IR-746 in (A) MeOH, (B) Mili-Q Water, (C) 0.9% brine, and (D) 10% FBS+Mili-Q Water, at different concentrations (0.5-20 μM).

FIG. 14A-14D. Photophysical properties of fluorophores SAT-IR-758 in (A) MeOH, (B) Mili-Q Water, (C) 0.9% brine, and (D) 10% FBS+Mili-Q Water, at different concentrations (0.5-20 μM).

FIG. 15A-15D. Photophysical properties of fluorophores SAT-IR-741 in (A) MeOH, (B) Mili-Q Water, (C) 0.9% brine, and (D) 10% FBS+Mili-Q Water, at different concentrations (0.5-20 μM).

FIG. 16A-16D. Photophysical properties of fluorophores SAT-IR-745 in a. MeOH, b. Mili-Q Water, c. 0.9% brine, and d. 10% FBS+Mili-Q Water, at different concentrations (0.5-20 μM).

FIG. 17A-17D. Photophysical properties of fluorophores SAT-IR-747 in (A) MeOH, (B) Mili-Q Water, (C) 0.9% brine, and (D) 10% FBS+Mili-Q Water, at different concentrations (0.5-20 μM).

FIG. 18A-18D. Photophysical properties of fluorophores SAT-IR-743 in (A) MeOH, (B) Mili-Q Water, (C) 0.9% brine, and (D) 10% FBS+Mili-Q Water, at different concentrations (0.5-20 μM).

FIG. 19A-19D. Photophysical properties of fluorophores SAT-IR-761 in (A) MeOH, (B) Mili-Q Water, (C) 0.9% brine, and (D) 10% FBS+Mili-Q Water, at different concentrations (0.5-20 μM).

FIG. 20A-20D. Photophysical properties of fluorophores SAT-IR-748 in (A) MeOH, (B) Mili-Q Water, (C) 0.9% brine, and (D) 10% FBS+Mili-Q Water, at different concentrations (0.5-20 μM).

FIG. 21A-21C. (A) absorbance of AlexaFluor 750 NHS Ester in organic solvents (methanol) and in aqueous (PBS pH 7.2, 10% FBS+Mili-Q Water, and) and (B) emission spectra of AlexaFlour750 organic solvents (methanol) and in aqueous (PBS pH 7.2, 10% FBS+Mili-Q Water, and) and (C) emission spectra of AlexaFluore750 NHS Ester conjugated to IgG. The absorbance and fluorescence were measured at concentrations of 1 μM.

FIG. 22A-22C. Bioconjugation of a secondary antibody, donkey anti-rabbit with (A) SAT-IR-746-NHS Ester (145 eq, 5.61 DOL), (B) Alexafluor 750 (20 eq, 4.82 DOL), (C) IRDYE800CWNHS Ester (20 eq, 5.43 DOL) at different equivalences.

FIG. 23. Photophysical properties of Symmetrical Oxonol Fluorophores.

FIG. 24. Photophysical properties of Diverse Pyrazoles containing Oxonol Fluorophores.

FIG. 25. Schematic of chemical reaction depicting how pyridinium benzoxazole enables polymethine modification.

FIG. 26. Schematic of chemical reaction depicting the synthesis of symmetric and unsymmetric Cy7.

FIG. 27A-27B. Photophysical properties of SAT-IR-740. (A) Chart of properties. (B) chemical structure.

FIG. 28A-28C. Absorbance of Cysteine Labeled IgG-SAT-IR-746. (A) Chemical structure of SAT-IR-746. (B) Data corresponding to the graph. (C) Graph of absorbance data.

FIG. 29. Serum stability of fluorogenic probe.

FIG. 30. Activation of dye by absorbance and fluorescence.

FIG. 31A-31B. BK10067 50 μM H₂O₂ Activation. (A) BK10067 activation (50 μM) in PBS 7.2 at 37° C. (B) BK10067 fluorogenic probe is highly selective for H₂O₂ compared to other intracellular analytes.

FIG. 32A-32B. Synthesis of off-dye BK10082 (A) UV-Vis Confirmation of Off-Dye. (B) Stability in Serum, most stable in 10% FBS medium retaining ˜80%, in pH 5.0 it retains ˜60%.

FIG. 33A-33B. Synthesis of off-dye BK10085 (A) UV-Vis Confirmation of Off-Dye. (B) Most stable at pH 5.0.

FIG. 34A-34B. Absorbance and fluorescence emission intensity BK10083. (A) Absorbance BK10083. (B) Fluorescence Emission Intensity BK10083.

FIG. 35A-35C. BK10083 Increase in Concentration of NTR enzyme Vs Absorbance @RT at 0 min. (A) Graph showing absorbance BK10083. (B) Table showing data corresponding to the graph. (C) Bar graph showing BK10083 increase in concentration of NTR enzyme Vs Absorbance @RT at 0 min.

FIG. 36A-36C. BK10083 Increase in Concentration of NTR enzyme Vs Fluorescence @RT at 0 min. (A) Graph showing fluorescence emission intensity of BK10083 (B) Table showing data corresponding to the graph (C) Bar graph showing BK10083 increase in concentration of NTR enzyme fluorescence @RT at 0 min.

FIG. 37A-37C. Increase in Concentration of NTR enzyme Vs Absorbance @heat at 37° C. for 1 hr. (A) Graph showing absorbance BK10083. (B) Table showing data corresponding to the graph. (C) Bar graph showing BK10083 increase in concentration of NTR enzyme Vs Absorbance @heat at 37° C. for 1 hr.

FIG. 38A-38C. Increase in Concentration of NTR enzyme Vs Fluorescence @heat at 37° C. for 1 hr. (A) Graph showing fluorescence emission intensity of BK10083 (B) Table showing data corresponding to the graph (C) Bar graph showing BK10083 increase in concentration of NTR enzyme fluorescence @heat at 37° C. for 1 hr.

FIG. 39A-39B. Absorbance and fluorescence emission intensity BK10085. (A) Absorbance. (B) Fluorescence.

FIG. 40A-40C. Increase in Concentration of NTR enzyme Vs Absorbance @RT at 0 min. (A) Graph showing absorbance BK10085. (B) Table showing data corresponding to the graph. (C) Bar graph showing BK10085 increase in concentration of NTR enzyme Vs Absorbance @RT at 0 min.

FIG. 41A-41C. Increase in Concentration of NTR enzyme Vs Fluorescence @RT at 0 min. (A) Graph showing fluorescence emission intensity of BK10085 (B) Table showing data corresponding to the graph (C) Bar graph showing BK10085 increase in concentration of NTR enzyme fluorescence @RT at 0 min.

FIG. 42A-42C. Increase in Concentration of NTR enzyme Vs Absorbance @heat at 37° C. for 1 hr. (A) Graph showing absorbance BK10085. (B) Table showing data corresponding to the graph. (C) Bar graph showing BK10085 increase in concentration of NTR enzyme Vs Absorbance @heat at 37° C. for 1 hr.

FIG. 43A-43C. Increase in Concentration of NTR enzyme Vs Fluorescence @heat at 37° C. for 1 hr. (A) Graph showing fluorescence emission intensity of BK10085 (B) Table showing data corresponding to the graph (C) Bar graph showing BK10085 increase in concentration of NTR enzyme fluorescence @heat at 37° C. for 1 hr.

FIG. 44A-44C. E.G.1040 stability in various solvents. (A) Data table showing absorbance and emission (B) Absorbance at various wavelengths (C) Fluorescence intensity over time.

FIG. 45. Photostability assay of E.G1040, DYIR800CW, and E.G1070

FIG. 46. General Approach applicable to other indoles depicting other R1 groups

FIG. 47. Photostability Study of SO3 CFXs dyes vs. AF647.

FIG. 48. SO3 CFXs Cy5 dyes Abs vs. time.

FIG. 49. SO3 CFXs emission spectra.

FIG. 50. SO3 CFXs DOL Vs. Fluorescence.

FIG. 51. SO3 CFXs mAb Vs. AF647 mAb.

FIG. 52. Photostability of CFX Cy3 dyes.

FIG. 53. Photostability of sulfonated CF3 and CHF2 Cy3 Dyes.

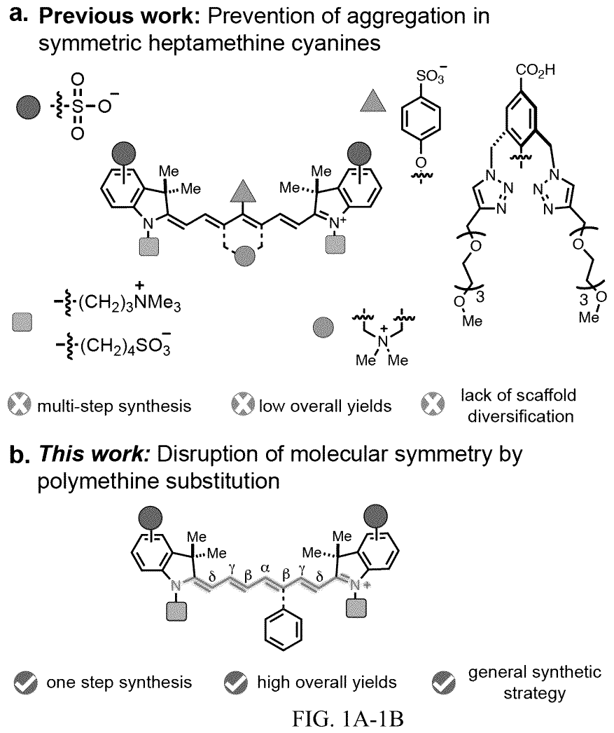

### DESCRIPTION

The following discussion is directed to various embodiments of the invention. The term “invention” is not intended to refer to any particular embodiment or otherwise limit the scope of the disclosure. The embodiments disclosed should not be interpreted, or otherwise used, as limiting the scope of the disclosure, including the claims. One skilled in the art will understand that the following description has broad application and the discussion of any embodiment is meant only to be an example of that embodiment and not intended to imply that the scope of the disclosure, including the claims, is limited to that embodiment.

Fluorophores significantly advance biomedical research by offering enhanced sensitivity and specificity for detecting and imaging cellular and molecular processes, enabling observation of previously undetectable subtle changes. These fluorophores exhibit superior photostability and brightness, yielding clearer, longer-lasting imaging results. They can be engineered to emit light in specific wavelength ranges, facilitating multiplexing for simultaneous imaging of multiple targets in a single sample. Additionally, they enable the design of targeted probes that selectively bind to specific biomolecules or cellular structures, supporting precise localization and visualization.

Oxonol and cyanine dyes share a conjugated polymethine chain (—CH═CH—)ₙ that enables fluorescence in the visible to near-infrared range, making them valuable for biomedical imaging applications like live-cell imaging and bioconjugation. Both are modified using a one-step PyBox-mediated synthesis to prevent aggregation, achieving high yields (e.g., 98% for oxonol BK10029, 55-80% for cyanine SAT-IR-746) and stability in physiological conditions.

A one-step synthesis method has been developed to produce non-aggregating fluorophores by modifying the polymethine chain of cyanine dyes. This approach is broadly applicable for creating non-aggregating fluorophores for biomolecule labeling. Additionally, the modified cyanine dyes can be synthesized with various functional groups across the polymethine chain for tuning of optical properties or bio-conjugation possibilities. The synthetic method is designed for scalability and industrial applicability, leveraging commercially available reagents like phenylacetaldehyde and pyridinium benzoxazole derivatives to ensure cost-effectiveness and accessibility. The method's flexibility in modifying the polymethine chain allows for tailored synthesis of dyes with specific reactive groups for bio-conjugation, enhancing their utility in targeted imaging applications, such as fluorescence-guided surgery, molecular diagnostics, and theranostics.

### I. CYANINES

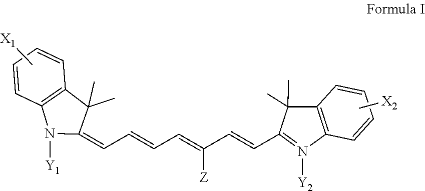

Cyanine dyes have the general structure R₂N(CH═CH)n-CH═NR₂. These dyes can be used as cellular membrane probes, DNA stains, or protein probes. Cyanine dye labels are named according to the number of carbons between the indoline moieties. One example is Formula I (Cy7) where X₁ and X₂ are independently selected from hydrogen (H), SO₃, C1-C3 alkyl, C1-C3 sulfonate; Y₁ and Y₂ are independently selected from C1-C4 alkyl, halogen (I, Br, Fl) substituted C1-C4 alkyl (e.g., CH2F, CHF2, or CF₃)SO₃, C1-C5 sulfonate, C3-C6 ethylene glycol, C1-C4 alkylamine; choline; and Z is a C1-C4 alkyl, C1-C4 alkoxy, carboxy, C1-C4 carboxamide, substituted phenylcarboxy (e.g., carboxyphenyl succinimide), substituted or unsubstituted alky, aryl (e.g., phenyl), or heteroatom moiety, the substituted alky, aryl (e.g., phenyl), or heteroatom moiety can have a substitution selected from halogen (e.g., fluoro, chloro, bromo, or iodo); C1-C6 alkyl, optionally substituted with one or more substituents selected from the group consisting of halogen, hydroxy, C1-C4 alkoxy, cyano, or amino; C2-C6 alkenyl, optionally substituted with one or more substituents selected from the group consisting of halogen, hydroxy, C1-C4 alkoxy, cyano, or amino; C2-C6 alkynyl, optionally substituted with one or more substituents selected from the group consisting of halogen, hydroxy, C1-C4 alkoxy, cyano, or amino; C1-C6 alkoxy, optionally substituted with one or more substituents selected from the group consisting of halogen, hydroxy, C1-C4 alkoxy, cyano, or amino; C1-C6 haloalkyl, including trifluoromethyl, difluoromethyl, or chloromethyl; C1-C6 haloalkoxy, including trifluoromethoxy or difluoromethoxy; hydroxy; cyano; nitro; amino, including —NH₂, —NH(C1-C4 alkyl), or —N(C1-C4 alkyl)₂; C1-C6 acyl, including acetyl or propionyl, optionally substituted with one or more substituents selected from the group consisting of halogen, hydroxy, or C1-C4 alkoxy; C1-C6 acyloxy, including acetoxy or propionyloxy; C1-C6 alkylsulfonyl, including methylsulfonyl or ethylsulfonyl; C1-C6 alkylsulfinyl, including methylsulfinyl or ethylsulfinyl; C1-C6 alkylthio, including methylthio or ethylthio; C6-C10 aryl, optionally substituted with one or more substituents selected from the group consisting of halogen, C1-C4 alkyl, C1-C4 alkoxy, hydroxy, cyano, nitro, or amino; C5-C10 heteroaryl, containing 1 to 4 heteroatoms independently selected from nitrogen, oxygen, or sulfur, optionally substituted with one or more substituents selected from the group consisting of halogen, C1-C4 alkyl, C1-C4 alkoxy, hydroxy, cyano, nitro, or amino; C3-C8 cycloalkyl, optionally substituted with one or more substituents selected from the group consisting of halogen, C1-C4 alkyl, C1-C4 alkoxy, hydroxy, cyano, or amino; C3-C8 heterocyclyl, containing 1 to 3 heteroatoms independently selected from nitrogen, oxygen, or sulfur, optionally substituted with one or more substituents selected from the group consisting of halogen, C1-C4 alkyl, C1-C4 alkoxy, hydroxy, cyano, or amino; Carboxyl, including COOH or salts thereof, C1-C6 alkoxycarbonyl, including methoxycarbonyl or ethoxycarbonyl; Aminocarbonyl, including —C(O)NH₂, —C(O)NH(C1-C4 alkyl), or —C(O)N(C1-C4 alkyl)₂; Sulfonylamino, including —SO₂NH₂, —SO₂NH(C1-C4 alkyl), or —SO₂N(C1-C4 alkyl)₂; and combinations thereof, wherein two or more substituents on the phenyl moiety may optionally be taken together to form a fused C5-C6 cycloalkyl, C5-C6 heterocyclyl, C6-C10 aryl, or C5-C10 heteroaryl ring, each of which may be optionally substituted with one or more substituents selected from the group consisting of halogen, C1-C4 alkyl, C1-C4 alkoxy, hydroxy, cyano, nitro, or amino. In certain embodiments, the phenyl moiety is unsubstituted. In other embodiments, the phenyl moiety is substituted with 1 to 5 substituents, preferably 1 to 3 substituents, selected from the above list. Each substituent is independently selected, and where a substituent is itself substituted, the substitutions are as defined above unless otherwise specified.

### A. Synthesis of Unsymmetrical Heptamethine Cyanines

The unsymmetrical heptamethine cyanines can be synthesized through selective substitution on the polymethine chain to explore the effect of molecular symmetry on aggregation. The San Antonio-near-infrared fluorophores (SAT-IR) series of near-infrared dyes and analogs thereof are typically synthesized through a process involving the initial preparation of indolium or quinolinium intermediates followed by their condensation to form the final polymethine structures. Three- or 4-phenyl substituted pyridines are reacted with 2-chlorobenzoxazole under nucleophilic substitution conditions to form pyridinium benzoxazole intermediates. Intermediates such as 1A, D, S4, S10, S12, S14, 2E, and 2F are generated by combining equimolar or excess amounts of starting compounds (e.g., A with C, S1 with S2 or S3, E with C, F with C) in acetonitrile within a microwave vial, followed by heating at 80-100° C. or reflux for 18 h; reactions were monitored by LC/MS, and products were isolated via precipitation with diethyl ether or purification using normal-phase CombiFlash ISCO chromatography (0-40% DCM:MeOH), yielding colored powders or liquids in 56-95% yields. Some intermediates (e.g., 1, S6, S8) were prepared according to established literature methods. The final dyes (e.g., SAT-IR-746, -745, -741, -747, -758, -743, -748, -761, -746-CO2H, -758-CO2H) were then obtained by condensing these intermediates (typically 1.0 eq of a central unit like 1A, 1B, S10, S12, S14, 2E, or 2F with 2.0-4.0 eq of a flanking component like D, S4, S6, or S8) in the presence of sodium acetate (2.0-4.0 eq) in solvents such as ethanol, acetonitrile, or acetic acid/acetic anhydride mixtures, under heating at 60-80° C. for 15-60 min. Crude mixtures were adsorbed onto silica, evaporated, and purified by normal-phase (0-40% DCM:MeOH) or reverse-phase (5-95% MeCN:H2O+0.05% FA) CombiFlash ISCO chromatography, affording the target compounds as blue or green powders in 52-82% yields. Reactive derivatives like SAT-IR-746-NHS and -758-NHS were further prepared from the corresponding carboxylic acids via activation with TSTU and DIPEA in DMF, followed by coupling with NHS and precipitation in diethyl ether, yielding 50-52%. All compounds were characterized by 1H and 13C NMR spectroscopy, LC/MS, and HRMS, confirming their structures and purities.

Pyridinium benzoxazoles. In certain aspects the pyridinium benzoxazoles have the general structure of Formula II:

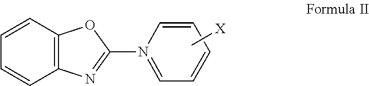

where X can be a substituted or unsubstituted aryl (e.g., phenyl), or heteroaryl moiety, the substituted aryl (e.g., phenyl), or heteroaryl moiety can have 1, 2, 3, 4, or 5 substitutions independently selected from halogen (e.g., fluoro, chloro, bromo, or iodo); C1-C6 alkyl, optionally substituted with one or more substituents selected from the group consisting of halogen, hydroxy, cyano, or amino; C2-C6 alkenyl, optionally substituted with one or more substituents selected from the group consisting of halogen, hydroxy, cyano, or amino; C2-C6 alkynyl, optionally substituted with one or more substituents selected from the group consisting of halogen, hydroxy, cyano, or amino; C1-C6 alkoxy, optionally substituted with one or more substituents selected from the group consisting of halogen, hydroxy, cyano, or amino; C1-C6 haloalkyl, including trifluoromethyl, difluoromethyl, or chloromethyl; C1-C6 haloalkoxy, including trifluoromethoxy or difluoromethoxy, hydroxy, cyano, nitro, amino (including —NH₂, —NH(C1-C4 alkyl), or —N(C1-C4 alkyl)₂); C1-C6 acyl, including acetyl or propionyl, optionally substituted with one or more substituents selected from the group consisting of halogen, hydroxy, or C1-C4 alkoxy; C1-C6 acyloxy, including acetoxy or propionyloxy; C1-C6 alkylsulfonyl, including methylsulfonyl or ethylsulfonyl; C1-C6 alkylsulfinyl, including methylsulfinyl or ethylsulfinyl; C1-C6 alkylthio, including methylthio or ethylthio; C6-C10 aryl, optionally substituted with one or more substituents selected from the group consisting of halogen, C1-C4 alkyl, C1-C4 alkoxy, hydroxy, cyano, nitro, or amino; C5-C10 heteroaryl, containing 1 to 4 heteroatoms independently selected from nitrogen, oxygen, or sulfur, optionally substituted with one or more substituents selected from the group consisting of halogen, C1-C4 alkyl, C1-C4 alkoxy, hydroxy, cyano, nitro, or amino; C3-C8 cycloalkyl, optionally substituted with one or more substituents selected from the group consisting of halogen, C1-C4 alkyl, C1-C4 alkoxy, hydroxy, cyano, or amino; C3-C8 heterocyclyl, containing 1 to 3 heteroatoms independently selected from nitrogen, oxygen, or sulfur, optionally substituted with one or more substituents selected from the group consisting of halogen, C1-C4 alkyl, C1-C4 alkoxy, hydroxy, cyano, or amino; carboxyl, including —COOH or salts thereof, C1-C6 alkoxycarbonyl, including methoxycarbonyl or ethoxycarbonyl; Aminocarbonyl, including —C(O)NH₂, —C(O)NH(C1-C4 alkyl), or —C(O)N(C1-C4 alkyl)₂; Sulfonylamino, including —SO₂NH₂, —SO₂NH(C1-C4 alkyl), or —SO₂N(C1-C4 alkyl)₂; and combinations thereof. In certain embodiments, a phenyl moiety is unsubstituted. In other embodiments, the phenyl moiety is substituted with 1 to 5 substituents, preferably 1 to 3 substituents, selected from the above list. Each substituent is independently selected, and where a substituent is itself substituted, the substitutions are as defined above unless otherwise specified.

Solvent. In certain aspects the solvent used is tetrahydrofuran (THF). THF is a polar aprotic solvent commonly used in organic synthesis due to its ability to dissolve a wide range of polar and non-polar compounds. It has a moderate boiling point (66° C.) and is particularly effective for reactions involving strong bases and supports low-temperature reactions. Alternatives to THF include but are not limited to diethyl Ether (Et₂O); 1,4-dioxane; toluene; dimethylformamide (DMF); or solvents with similar characteristics.

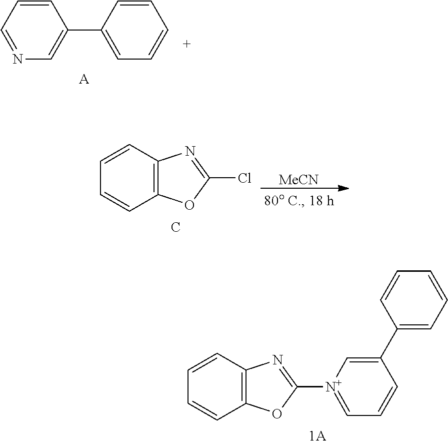

Compounds A (1.0 eq, 1.3 mmol) and C (1.0 eq, 1.3 mmol) were combined in a 20 mL microwave vial, followed by the addition of acetonitrile (3.0 mL). The reaction mixture was refluxed for 18 h. The reaction was precipitated using diethyl ether to give compound 1A as white powder (251.80 mg, 71%). ¹H NMR (500 MHz, DMSO) δ 9.97 (d, J=1.9 Hz, 1H), 9.92 (d, J=6.3 Hz, 1H), 9.43-9.41 (m, 1H), 8.58 (dd, J=8.2, 6.3 Hz, 1H), 8.06 (dq, J=7.4, 1.7 Hz, 4H), 7.71-7.62 (m, 5H). ¹³C NMR (126 MHz, DMSO) δ 153.62, 150.47, 149.25, 141.01, 139.97, 139.91, 139.83, 139.83, 139.74, 132.93, 131.08, 130.12, 129.03, 128.48, 128.37, 128.35, 127.26, 121.59, 112.51. LC/MS calculated for C₁₈H₁₃N₂O⁺=273.1; observed 273.2.

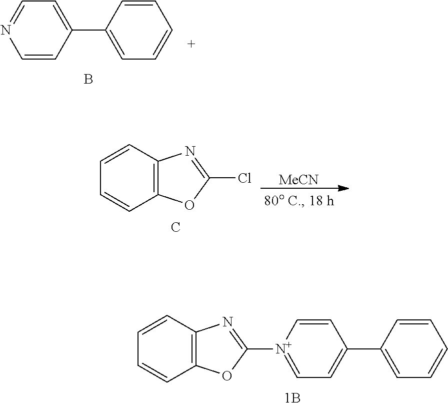

Compound 1B was synthesized according to literature procedure (Usama, J. Am. Chem. Soc. 2021, 143(15), 5674-79).

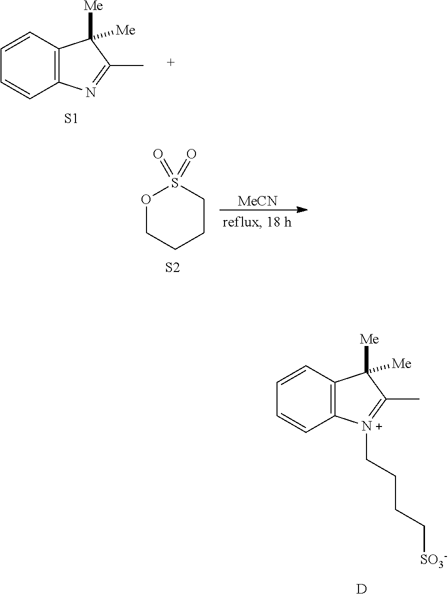

Compounds S1 (1.0 eq, 12.6 mmol) and S2 (3.0 eq, 37.7 mmol) were combined in a 20 mL microwave vial, followed by the addition of acetonitrile (2.0 mL). The reaction mixture was refluxed for 18 h. The reaction was precipitated using diethyl ether and vacuum-dried overnight to give compound D as pink powder (1.99 g, 56%). ¹H NMR (500 MHz, MeOD) δ 8.02-7.98 (m, 1H), 7.83-7.80 (m, 1H), 7.70-7.66 (m, 2H), 4.64-4.60 (m, 2H), 2.92 (t, J=7.3 Hz, 3H), 2.23-2.15 (m, 2H), 1.99 (p, J=7.4 Hz, 2H), 1.65 (s, 6H). ¹³C NMR (125 MHz, MeOD) δ 196.85, 142.06, 141.25, 129.78, 129.21, 123.30, 115.44, 54.60, 50.06, 47.79, 47.62, 26.18, 22.02, 21.58. LC/MS calculated for C₁₅H₂₁NO₃S=295.1; observed (M−1) 294.2.

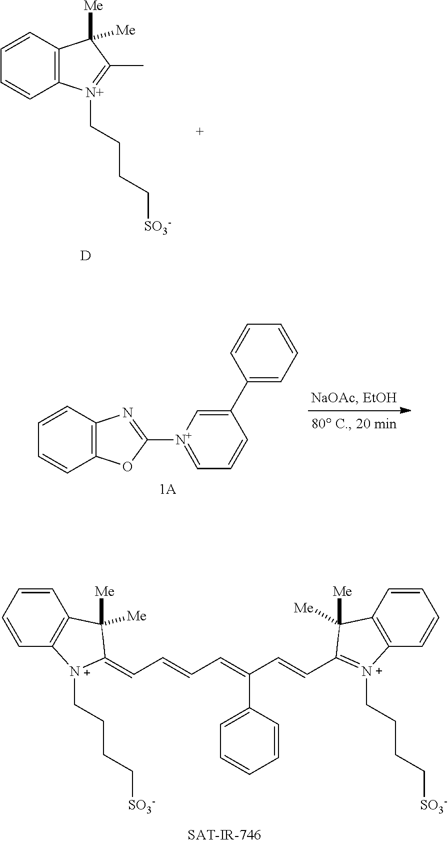

SAT-IR-746. Compounds D (2.5 eq,0.46 mmol) and 1A (1.0 eq, 0.18 mmol) were combined in a 20 mL microwave vial, followed by the addition of sodium acetate (2.5 eq, 0.46 mmol) and ethanol (2.0 mL). The reaction mixture was heated at 80° C. for 20 minutes. Silica was added to the reaction mixture, and the solvent was removed via rotary evaporation. The crude reaction mixture was purified to give compound SAT-IR-746 as blue powder (72 mg, 55%). ¹H NMR (300 MHz, MeOD) δ 8.05 (dd, J=18.6, 13.4 Hz, 2H), 7.79 (d, J=14.1 Hz, 1H), 7.60 (t, J=7.4 Hz, 2H), 7.48 (dd, J=11.4, 7.2 Hz, 3H), 7.43-7.36 (m, 2H), 7.36-7.28 (m, 3H), 7.28-7.18 (m, 3H), 6.32 (d, J=12.9 Hz, 2H), 5.76 (d, J=12.9 Hz, 1H), 4.60 (s, 1H), 4.10 (s, 2H), 3.79 (s, 2H), 3.37 (s, 1H), 2.85 (t, J=6.6 Hz, 2H), 2.77 (d, J=7.1 Hz, 2H), 1.93-1.87 (m, 3H), 1.75 (s, 6H), 1.72 (s, 7H). ¹³C NMR (126 MHz, DMSO) δ 173.45, 170.16, 153.65, 149.14, 142.78, 142.36, 141.00, 136.13, 135.01, 130.23, 129.50, 129.00, 128.86, 128.25, 125.66, 124.56, 123.89, 122.87, 122.77, 112.22, 111.05, 105.92, 100.41, 51.27, 51.24, 49.55, 48.76, 44.14, 43.65, 27.78, 27.66, 27.64, 27.61, 27.54, 27.49, 26.79, 26.28, 22.88, 22.70, 1.62. HRMS (ESI) calculated for C₄₁H₄₇N₂O₆S₂⁻, 727.2881 observed, (M⁻¹) 727.2880

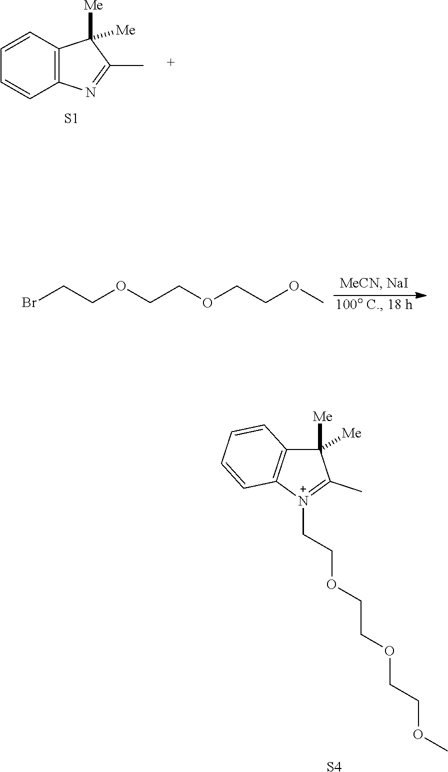

Compounds S1 (1.0 eq, 6.3 mmol) and S3 (1.5 eq, 9.4 mmol) were combined in a 20 mL microwave vial, followed by the addition of acetonitrile (2.0 mL) and sodium iodide (0.5 eq, 3.1 mmol). The reaction mixture was heated at 100° C. for 18 h. The crude reaction mixture was purified using normal phase Combi Flash ISCO purification (0-40% DCM:MeOH) to give compound S4 as wine red liquid (1.2 g, 62%). ¹H NMR (500 MHz, MeOD) δ 7.95-7.87 (m, 1H), 7.83-7.78 (m, 1H), 7.70-7.63 (m, 2H), 4.83-4.75 (m, 2H), 4.04-3.98 (m, 2H), 3.61-3.57 (m, 2H), 3.54-3.52 (m, 2H), 3.49 (s, 2H), 3.44 (s, 2H), 3.32 (d, J=4.3 Hz, 5H), 2.06 (s, 1H), 1.64 (s, 6H). ¹³C NMR (125 MHz, MeOD) δ 198.75, 141.91, 140.95, 129.68, 129.00, 123.25, 115.16, 115.13, 71.46, 71.43, 71.41, 70.14, 70.09, 70.04, 69.75, 69.72, 66.75, 57.63, 57.60, 54.67, 21.57, 21.55, 21.52, 21.50, 21.45, 21.40, 21.38, 21.36. LC/MS calculated for C₁₈H₂₈NO₃⁺=306.2; observed (M−1) 306.3.

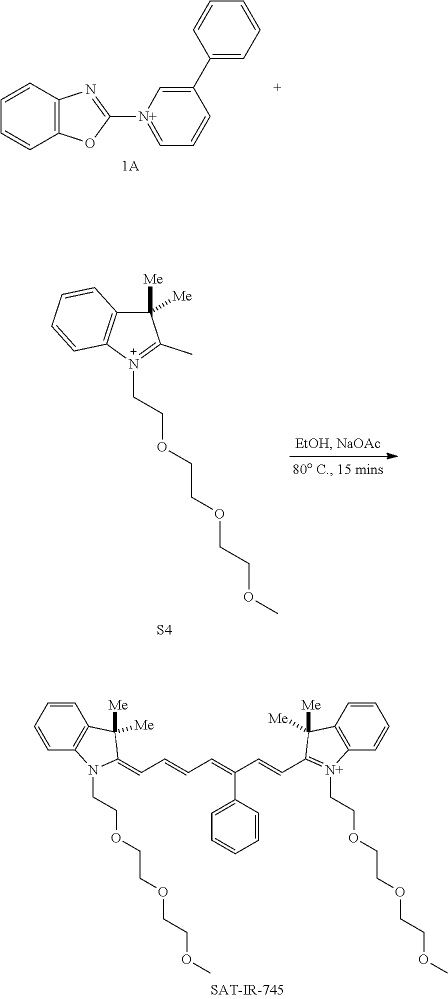

SAT-IR-745. Compounds 1A (1.0 eq, 0.18 mmol) and S4 (2.5 eq, 0.46 mmol) were combined in a 20 mL microwave vial, followed by the addition of sodium acetate (2.5 eq, 0.46 mmol) and ethanol (2.0 mL). The reaction mixture was heated at 80° C. for 15 minutes. Silica was added to the reaction mixture, and the solvent was removed via rotary evaporation. The crude reaction mixture was purified using reversed phase (5-95% MeCN:H₂O+0.05% FA) Combi Flash ISCO purification to give SAT-IR-745 as a blue powder (95 mg, 70%). ¹H NMR (300 MHz, MeOD) δ 8.03 (d, J=13.5 Hz, 2H), 7.78 (d, J=13.1 Hz, 1H), 7.57 (t, J=7.3 Hz, 3H), 7.49 (dd, J=7.3, 5.3 Hz, 4H), 7.44-7.36 (m, 2H), 7.36-7.29 (m, 4H), 7.29-7.20. (m, 3H), 6.35-6.17 (m, 2H), 5.92 (d, J=13.9 Hz, 1H), 4.27 (t, J=5.4 Hz, 1H), 3.99 (t, J=5.5 Hz, 2H), 3.82 (t, J=5.2 Hz, 2H), 3.68 (t, J=5.2 Hz, 2H), 3.54 (dd, J=6.2, 2.9 Hz, 2H), 3.50 (s, 1H), 3.49-3.46 (m, 6H), 3.43 (q, J=3.3 Hz, 4H), 3.38 (dd, J=6.1, 3.1 Hz, 2H), 3.30 (d, J=4.0 Hz, 6H), 1.77 (s, 6H), 1.73 (s, 6H). ¹³C NMR (125 MHz, MeOD) δ 173.58, 171.97, 152.46, 149.95, 142.62, 142.55, 141.11, 140.73, 136.21, 135.70, 129.85, 128.74, 128.21, 128.15, 127.59, 124.92, 124.40, 123.79, 121.83, 111.35, 110.62, 71.56, 71.54, 71.49, 71.46, 71.44, 71.42, 70.65, 70.62, 70.57, 70.45, 70.30, 70.25, 70.19, 70.01, 67.72, 67.27, 57.70, 49.17, 48.77, 44.17, 43.81, 26.71, 26.60, 21.39. HRMS (ESI) calculated for C₄₇H₆₁N₂O₆⁺, 749.4524, observed (M⁺¹) 749.4523.

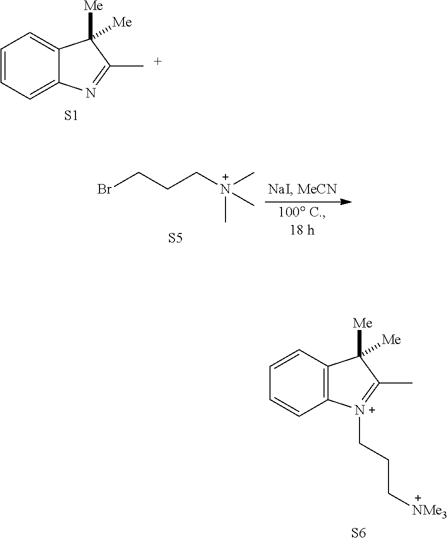

Compound S6 was synthesized using literature procedures (Atkinson, Org. Biomol. Chem. 2021, 19(18), 4100-06).

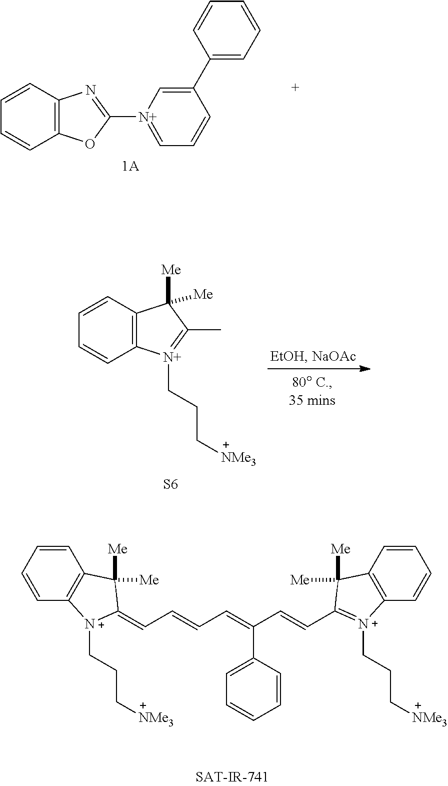

Compounds 1A (2.5 eq, 0.92 mmol) and S6 (1.0 eq, 0.37 mmol) were combined in a 20 mL microwave vial, followed by the addition of sodium acetate (2.5 eq, 0.92 mmol) and ethanol (2.0 mL). The reaction mixture was heated at 80° C. for 35 minutes. Silica was added to the reaction mixture, and the solvent was removed via rotary evaporation. The crude reaction mixture was purified using normal phase Combi Flash ISCO (0-40% DCM:MeOH) to give compound SAT-IR-741 as a blue powder (220 mg, 74%). ¹H NMR (500 MHz, MeOD) δ 8.16 (d, J=13.0 Hz, 1H), 8.13-8.07 (m, 1H), 7.88 (d, J=13.2 Hz, 1H), 7.60 (t, J=7.5 Hz, 2H), 7.56 (d, J=7.5 Hz, 1H), 7.52 (dd, J=7.3, 5.4 Hz, 2H), 7.46 (t, J=7.7 Hz, 1H), 7.40 (d, J=7.8 Hz, 2H), 7.35 (s, 1H), 7.33 (d, J=7.4 Hz, 2H), 7.27 (d, J=3.4 Hz, 1H), 7.27-7.25 (m, 1H), 6.36 (d, J=13.7 Hz, 1H), 6.29 (t, J=12.8 Hz, 1H), 5.72 (d, J=13.6 Hz, 1H), 4.14 (t, J=7.7 Hz, 2H), 3.88 (t, J=7.6 Hz, 2H), 3.53-3.49 (m, 2H), 3.31-3.28 (m, 2H), 3.14 (s, 9H), 3.08 (s, 9H), 2.27-2.20 (m, 2H), 2.11-2.05 (m, 2H), 1.79 (s, 6H), 1.77 (s, 6H). ¹³C NMR (125 MHz, MeOD) δ 174.26, 170.94, 167.98, 154.36, 149.54, 141.94, 141.55, 141.47, 140.65, 134.67, 130.20, 128.67, 128.44, 125.77, 124.65, 124.27, 123.72, 122.32, 122.19, 122.00, 110.91, 109.80, 109.47, 109.24, 105.26, 99.70, 63.17, 62.95, 52.37, 52.34, 52.31, 52.28, 52.25, 52.22, 49.65, 48.80, 48.78, 40.54, 40.04, 26.69, 26.55, 26.49, 20.97, 20.93, 20.47. HRMS (ESI) calculated for C₄₅H₆₁N₄³⁺, 657.4880; observed (M⁺³) 219.1625.

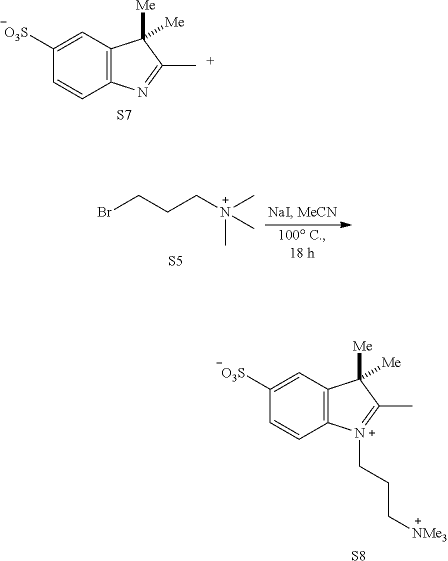

Compound S8 was synthesized using literature procedures (Atkinson, Org. Biomol. Chem. 2021, 19(18), 4100-06).

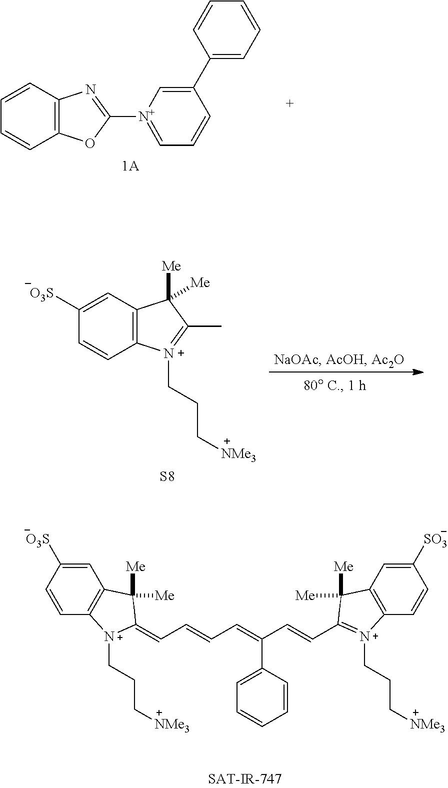

SAT-IR-747. Compounds 1A (1.0 eq, 0.73 mmol) and S8 (4.0 eq, 2.92 mmol) were combined in a 20 mL microwave vial, followed by the addition of sodium acetate (4.0 eq, 2.92 mmol) and Acetic acid and Acetic anhydride (1 mL each). The reaction mixture was heated at 60° C. for 60 minutes. The reaction was mixed with silica and the solvent was removed to get a powder form mixture. The crude powder reaction mixture was purified using reversed phase Combi Flash ISCO purification to give compound SATIR-747 as blue powder (321 mg, 54%). ¹H NMR (500 MHz, MeOD) δ 8.22-8.10 (m, 4H), 7.92 (d, J=1.7 Hz, 1H), 7.89 (d, J=1.7 Hz, 1H), 7.80 (ddd, J=20.7, 8.3, 1.7 Hz, 3H), 7.61 (t, J=7.6 Hz, 2H), 7.55-7.51 (m, 2H), 7.40 (d, J=8.4 Hz, 1H), 7.37-7.34 (m, 2H), 7.27 (d, J=8.4 Hz, 1H), 6.40-6.30 (m, 3H), 4.14 (t, J=7.7 Hz, 2H), 3.89 (t, J=7.7 Hz, 2H), 3.52-3.48 (m, 2H), 3.15 (s, 9H), 3.10 (s, 10H), 1.78 (d, J=15.2 Hz, 12H). ¹³C NMR (125 MHz, MeOD) δ 174.10, 170.83, 163.53, 160.30, 160.02, 154.54, 143.33, 142.99, 141.29, 140.64, 129.84, 128.99, 127.95, 126.78, 126.65, 125.92, 124.15, 123.79, 120.10, 119.14, 114.24, 112.26, 110.41, 109.53, 105.48, 101.12, 63.13, 62.95, 52.37, 49.45, 48.76, 40.41, 35.62, 29.34, 26.62, 26.50, 22.33, 21.26, 20.92, 20.45. HRMS (ESI) calculated for C₄₅H₅₉N₄O₆S₂⁺, 815.3871; observed (M⁺) 815.3866.

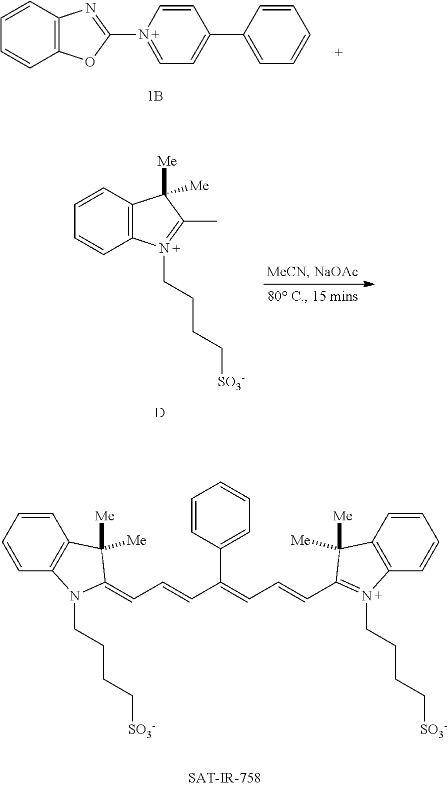

SAT-IR-758. Compounds 1B (1.0 eq, 1.7 mmol) and D (2.0 eq, 3.4 mmol) were combined in a 20 mL microwave vial, followed by the addition of sodium acetate (2.5 eq, 4.25 mmol) and acetonitrile (2.0 mL). The reaction mixture was heated at 80° C. for 15 minutes. Silica was added to the reaction mixture, and the solvent was removed via rotary evaporation. The crude reaction mixture was purified using normal phase Combi Flash ISCO (0-40% DCM:MeOH) to give compound SAT-IR-758 as blue powder (900 mg, 73%). ¹H NMR (500 MHz, MeOD) δ 7.67 (p, J=6.5 Hz, 3H), 7.48 (d, J=14.2 Hz, 2H), 7.40 (q, J=6.6 Hz, 6H), 7.33 (t, J=7.0 Hz, 2H), 7.23 (q, J=6.9 Hz, 2H), 6.83 (dd, J=13.1, 5.4 Hz, 2H), 6.44 (dd, J=13.7, 5.4 Hz, 2H), 4.14 (t, J=6.9 Hz, 4H), 2.93 (q, J=6.5 Hz, 4H), 2.06-1.88 (m, 9H), 1.36 (d, J=5.4 Hz, 12H). 13C NMR (125 MHz, MeOD) δ 172.42, 165.08, 149.64, 143.40, 141.86, 130.12, 129.91, 129.81, 126.87, 125.84, 123.64, 112.43, 105.25, 61.87, 52.00, 49.55, 44.84, 33.15, 28.48, 27.42, 23.69. HRMS (ESI) calculated for C₄₁H₄₇N₂O₆S₂⁻, 727.2881; observed (M⁻¹) 727.2877.

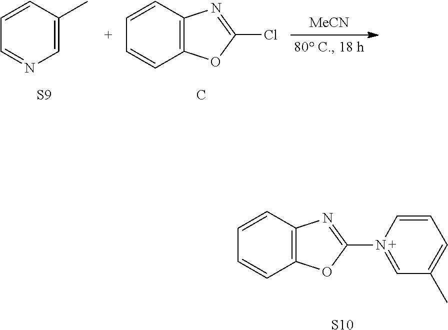

Compounds S9 (1.0 eq, 10.7 mmol) and C (1.0 eq, 10.7 mmol) were combined in a 20 mL microwave vial, followed by the addition of acetonitrile (2.0 mL). The reaction mixture was heated at 80° C. for 18 h. The crude reaction mixture was purified using normal phase Combi Flash ISCO purification (0-40% DCM:MeOH) to give compound S10 as white fine powder (1.9 g, 94%). ¹H NMR (500 MHz, MeOD) δ 9.84 (s, 1H), 9.78 (dd, J=6.4, 1.7 Hz, 1H), 8.86 (d, J=8.0 Hz, 1H), 8.34 (dd, J=8.1, 6.3 Hz, 1H), 7.98 (dd, J=7.9, 1.3 Hz, 1H), 7.94-7.90 (m, 1H), 7.69 (td, J=7.9, 1.4 Hz, 1H), 7.64 (td, J=7.7, 1.2 Hz, 1H), 2.78 (s, 3H). ¹³C NMR (125 MHz, MeOD) δ 151.56, 150.75, 140.65, 140.55, 139.80, 138.77, 127.90, 127.60, 126.59, 121.05, 111.39, 17.21. LC/MS calculated for C₁₃H₁₁N₂O⁺=211.2; observed (M⁺¹) 211.1.

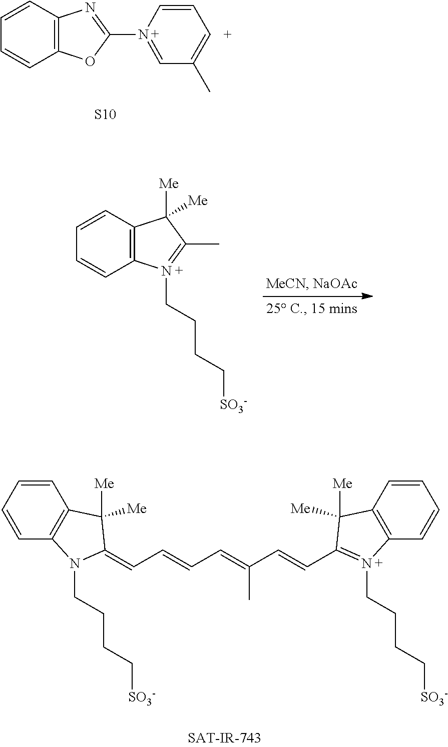

Compounds S10 (1.0 eq, 0.47 mmol) and D (4.0 eq, 1.88 mmol) were combined in a 20 mL microwave vial, followed by the addition of sodium acetate (4.0 eq, 1.88 mmol) and acetonitrile (2.0 mL). The reaction mixture was stirred at room temperature for 15 minutes. Silica was added to the reaction mixture, and the solvent was removed via rotary evaporation. The crude reaction mixture was purified using normal phase Combi Flash ISCO (0-40% DCM:MeOH) followed by reverse phase Combi Flash ISCO (0-50% water:acetonitrile) to give compound SAT-IR-743 as green powder (251 mg, 80%). ¹H NMR (500 MHz, MeOD) δ 7.96 (t, J=12.8 Hz, 1H), 7.83 (d, J=13.8 Hz, 1H), 7.47 (d, J=7.5 Hz, 2H), 7.41 (t, J=7.7 Hz, 2H), 7.30 (dd, J=8.1, 3.9 Hz, 2H), 7.24 (t, J=7.5 Hz, 2H), 6.72 (t, J=12.6 Hz, 1H), 6.37 (d, J=13.5 Hz, 1H), 6.20 (d, J=13.7 Hz, 1H), 4.14 (dt, J=19.7, 6.7 Hz, 5H), 3.33 (s, 1H), 2.92 (t, J=6.9 Hz, 5H), 2.08 (s, 3H), 1.69 (s, 6H), 1.68 (s, 6H). ¹³C NMR (125 MHz, MeOD) δ 171.73, 157.69, 152.02 (d, J=39.3 Hz), 142.26 (d, J=4.4 Hz), 141.02 (d, J=9.0 Hz), 129.74, 128.50, 124.67 (d, J=4.6 Hz), 122.32 (d, J=72.3 Hz), 110.54, 103.69, 99.88, 50.53, 48.93, 43.38, 26.78 (d, J=11.0 Hz), 25.90 (d, J=18.5 Hz), 22.15 (d, J=10.8 Hz), 10.55. HRMS (ESI) calculated for C₃₆H₄₅N₂O₆S₂⁻, 667.2870; observed (M⁺¹) 667.2870.

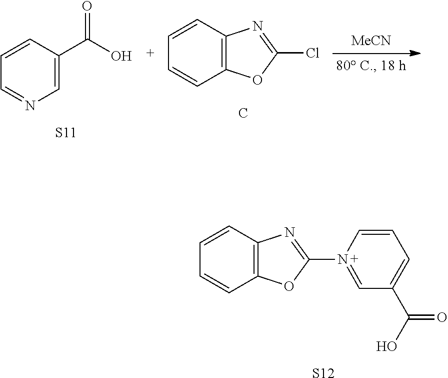

Compounds S11 (1.0 eq, 8.1 mmol) and C (1.5 eq, 12.2 mmol) were combined in a 20 mL microwave vial, followed by the addition of acetonitrile (2.0 mL). The reaction mixture was heated at 80° C. for 18 h. The crude reaction mixture was purified using normal phase Combi Flash ISCO purification (0-40% DCM:MeOH) to give compound S12 as a light tan powder (3.7 g, 95%). ¹H NMR (500 MHz, MeOD) δ 10.19 (s, 1H), 10.13 (d, J=6.3 Hz, 1H), 9.46 (d, J=8.0 Hz, 1H), 8.58 (t, J=7.2 Hz, 1H), 8.01 (d, J=7.9 Hz, 1H), 7.96 (d, J=8.2 Hz, 1H), 7.72 (t, J=7.8 Hz, 1H), 7.66 (t, J=7.7 Hz, 1H). ¹³C NMR (125 MHz, MeOD) δ 162.94, 161.65, 152.44, 150.59, 143.80, 142.20, 139.74, 132.06, 128.62, 128.21, 126.78, 121.23, 111.52. LC/MS calculated for C₁₃H₉N₂O₃⁺=241.2; observed (M⁻¹) 241.1.

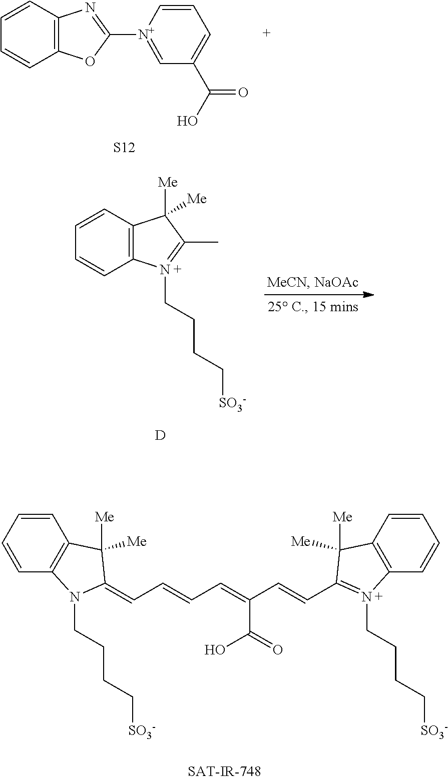

Compounds S12 (1.0 eq, 0.40 mmol) and D (4.0 eq, 1.60 mmol) were combined in a 20 mL microwave vial, followed by the addition of sodium acetate (4.0 eq, 1.60 mmol) and acetonitrile (2.0 mL). The reaction mixture was stirred at room temperature for 15 minutes. Silica was added to the reaction mixture, and the solvent was removed via rotary evaporation. The crude reaction mixture was purified using normal phase Combi Flash ISCO (0-40% DCM:MeOH) followed by reverse phase Combi Flash ISCO (0-50% water:acetonitrile) to give compound SAT-IR-748 as green powder (198 mg, 71%). ¹H NMR (500 MHz, D2O) δ 7.88 (t, J=13.0 Hz, 1H), 7.63 (d, J=14.2 Hz, 1H), 7.42 (dd, J=14.3, 7.4 Hz, 2H), 7.34 (dt, J=13.7, 7.7 Hz, 2H), 7.26-7.21 (m, 2H), 7.16 (t, J=7.7 Hz, 2H), 6.58 (d, J=13.1 Hz, 1H), 6.29 (d, J=13.7 Hz, 1H), 6.17 (d, J=14.1 Hz, 1H), 4.05-4.00 (m, 2H), 3.97 (t, J=7.3 Hz, 2H), 2.85 (t, J=7.6 Hz, 4H), 1.86-1.83 (m, 2H), 1.82 (s, 2H), 1.76 (d, J=9.4 Hz, 4H), 1.58 (s, 12H). ¹³C NMR (125 MHz, D2O) δ 181.50, 175.62, 173.64, 171.93, 153.24, 146.55, 142.42, 142.08, 141.63, 141.24, 128.52, 128.43, 125.23, 124.46, 122.28, 122.21, 111.23, 110.60, 50.48, 50.42, 49.28, 48.71, 43.52, 43.18, 26.94, 26.67, 25.70, 25.56, 23.21, 21.67, 21.61. HRMS (ESI) calculated for C₃₆H₄₅N₂O₈S₂⁻, 695.2455; observed (M⁻¹) 695.2458.

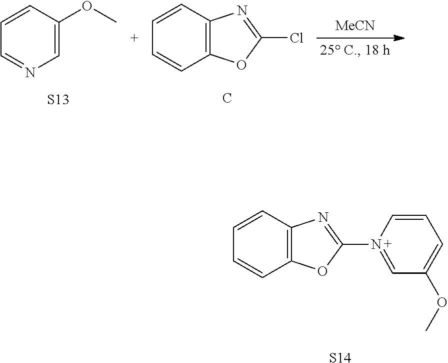

Compounds S13 (1.0 eq, 9.1 mmol) and C (1.0 eq, 9.1 mmol) were combined in a 20 mL microwave vial, followed by the addition of acetonitrile (2.0 mL). The reaction mixture was heated at 80° C. for 18 h. The crude reaction mixture was purified using normal phase Combi Flash ISCO purification (0-40% DCM:MeOH) to give compound S12 as a light white powder (1.9 g, 92%). ¹H NMR (500 MHz, MeOD) δ 9.59 (dt, J=6.2, 1.3 Hz, 1H), 9.55 (dd, J=2.7, 1.5 Hz, 1H), 8.65 (ddd, J=8.8, 2.7, 0.9 Hz, 1H), 8.38 (dd, J=8.9, 6.1 Hz, 1H), 8.00-7.97 (m, 1H), 7.94 (dt, J=8.3, 0.9 Hz, 1H), 7.70 (ddd, J=8.4, 7.4, 1.3 Hz, 1H), 7.64 (td, J=7.8, 1.2 Hz, 1H), 4.26 (s, 3H). ¹³C NMR (125 MHz, MeOD) δ 159.28, 150.80, 139.77, 135.75, 133.97, 128.81, 128.67, 127.99, 126.63, 121.09, 111.48, 57.29. LCMS (ESI) calculated for C₁₃H₁₁N₂O₂⁺, 227.0; observed (M⁺¹) 227.1.

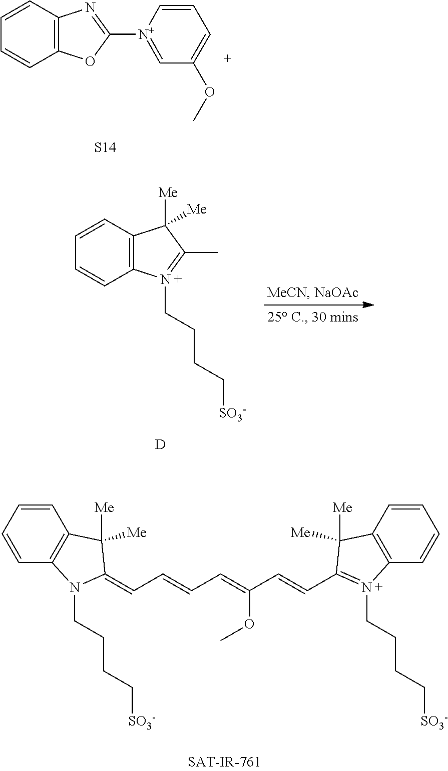

SAT-IR-761. Compounds S14 (1.0 eq, 0.25 mmol) and D (4.0 eq, 1.0 mmol) were combined in a 20 mL microwave vial, followed by the addition of sodium acetate (4.0 eq, 1.0 mmol) and acetonitrile (2.0 mL). The reaction mixture was stirred at room temperature for 30 minutes. Silica was added to the reaction mixture, and the solvent was removed via rotary evaporation. The crude reaction mixture was purified using normal phase Combi Flash ISCO (0-40% DCM:MeOH) followed by reverse phase Combi Flash ISCO (0-50% water:acetonitrile) to give compound SAT-IR-761 as green powder (88 mg, 52%). ¹H NMR (500 MHz, DMSO) δ 7.96 (s, 1H), 7.61 (d, J=7.6 Hz, 1H), 7.55 (d, J=7.4 Hz, 1H), 7.48 (d, J=8.1 Hz, 1H), 7.44-7.39 (m, 2H), 7.37 (d, J=4.4 Hz, 2H), 7.27 (t, J=7.4 Hz, 1H), 6.75-6.58 (m, 3H), 6.19-6.13 (m, 1H), 4.11 (dt, J=28.0, 7.3 Hz, 4H), 3.75 (s, 3H), 2.90 (s, 1H), 2.74 (s, 1H), 1.79 (dt, J=16.9, 7.7 Hz, 6H), 1.65 (s, 6H), 1.63 (s, 6H). ¹³C NMR (125 MHz, DMSO) δ 162.80, 154.49, 153.10, 150.85, 145.63, 142.92, 142.47, 141.80, 141.15, 128.89, 127.86, 125.55, 124.59, 122.88, 112.11, 111.19, 51.81, 51.32, 51.20, 49.46, 44.28, 43.76, 36.26, 31.25, 29.69, 29.34, 28.73, 27.58, 26.76, 26.60, 25.08, 22.90. HRMS (ESI) calculated for C₃₆H₄₅N₂O₈S₂⁻, 681.2663; observed (M−1) 681.2678.

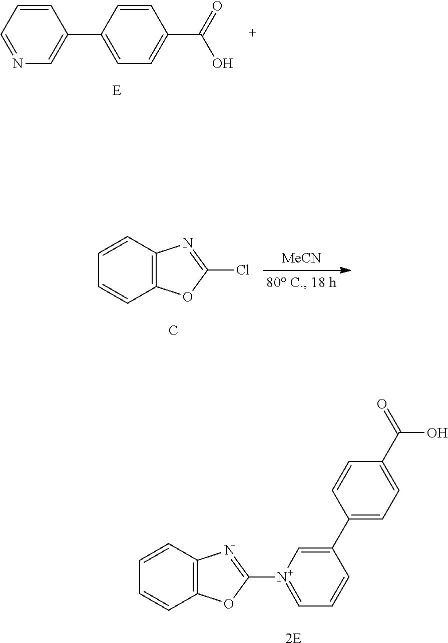

Compounds E (1.0 eq, 4.5 mmol) and C (1.0 eq, 4.5 mmol) were combined in a 20 mL microwave vial, followed by the addition of acetonitrile (9.0 mL). The reaction mixture was heated at 80° C. for 18 h. The crude reaction mixture was purified by precipitation in diethyl ether. Recrystallization was performed for further purification. Briefly, the precipitated compound (50 mg) was dissolved in acetonitrile (1.0 mL) in a 20 mL microwave vial and heated at 80° C. for 30 min. Milli-Q water (2 mL) was added while the compound cooling down to room temperature. The crude reaction mixture was purified by precipitation using diethyl ether to give compound 2E as white powder (1.03 g, 72%). ¹H NMR (500 MHz, DMSO) δ 8.82 (d, J=8.1 Hz, 2H), 8.11 (d, J=8.1 Hz, 4H), 8.05 (d, J=8.1 Hz, 2H), 8.00 (d, J=8.1 Hz, 3H), 7.87 (d, J=8.1 Hz, 1H). ¹³C NMR (125 MHz, DMSO) δ 167.48, 167.29, 143.56, 143.33, 142.38, 141.98, 139.02, 137.54, 131.89, 130.81, 130.60, 128.11, 127.64, 127.17, 124.23, 122.28, 110.27, 109.95. LC/MS calculated for C₁₉H₁₃N₂O₃⁺=317.1; observed (M) 317.2.

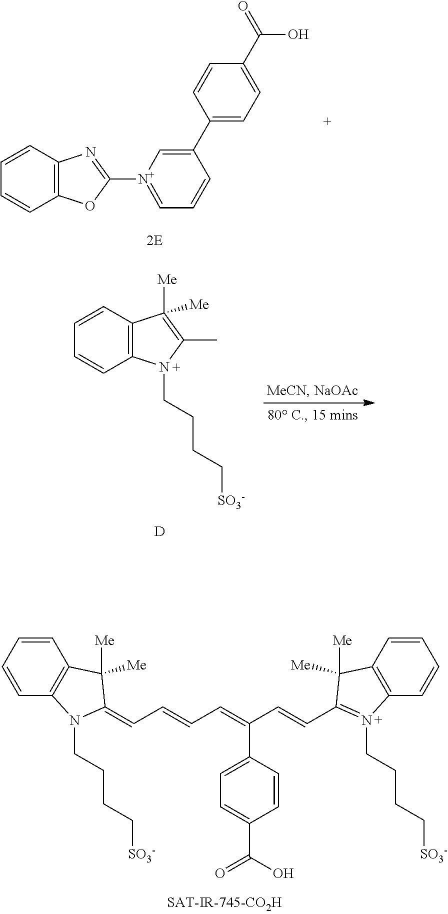

SAT-IR-746-CO₂H. Compounds 2E (1.0 eq,0.32) and D (2.5 eq, 0.79 mmol) were combined in a 20 mL microwave vial, followed by the addition of sodium acetate (2.5 eq, 0.79 mmol) and acetonitrile (2.0 mL). The reaction mixture was heated at 80° C. for 15 minutes. Silica was added to the reaction mixture, and the solvent was removed via rotary evaporation. The crude reaction mixture was purified using normal phase Combi Flash ISCO (0-40% DCM:MeOH) to give compound SAT-IR-746-CO₂H as blue powder (140 mg, 82%). ¹H NMR (500 MHz, MeOD) δ 8.13 (d, J=7.3 Hz, 1H), 7.95 (s, 1H), 7.74 (t, J=7.7 Hz, 1H), 7.58 (d, J=7.4 Hz, 1H), 7.51 (d, J=7.3 Hz, 1H), 7.45 (t, J=6.9 Hz, 1H), 7.41 (d, J=7.5 Hz, 1H), 7.36 (t, J=7.8 Hz, 2H), 7.22 (s, 1H), 4.11 (s, 1H), 3.78 (s, 1H), 2.86 (t, J=6.7 Hz, 2H), 2.80 (t, J=7.1 Hz, 2H), 1.90 (p, J=7.2 Hz, 3H), 1.75 (s, 6H), 1.72 (s, 6H). ¹³C NMR (126 MHz, MeOD) δ 168.03, 141.47, 140.75, 131.43, 129.27, 128.91, 128.38, 125.27, 124.29, 122.01, 110.21, 50.46, 49.38, 48.60, 43.16, 26.65, 22.17, 22.01. HRMS (ESI) calculated for C₄₂H₄₇N₂O₈S₂⁻, 771.2779; observed (M⁻¹) 771.2773.

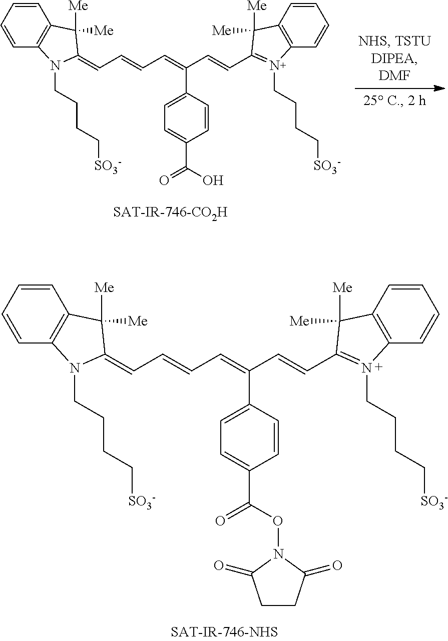

SAT-IR-746-NHS. SAT-IR-746-CO2H (1.0 eq, 0.01 mmol) was added to DMF (0.50 mL) in a 20 mL microwave vial, followed by the addition of TSTU (2.0 eq, 0.02 mmol) and DIPEA (1.5 eq, 0.02 mmol). The reaction mixture was stirred at 25° C. for 0.5 hours and then the addition of NHS (2.0 eq, 0.02 mmol). The reaction mixture was stirred at 25° C. for 2.0 h. The crude reaction mixture was purified by precipitating it in ether. Compound was dried by vacuum overnight to give compound SAT-IR-746-NHS as blue powder (9 mg, 52%). HRMS (ESI) calculated for C₄₆H₅₀N₃O₁₀S₂⁻, 868.2943; observed (M⁺²) 870.3075.

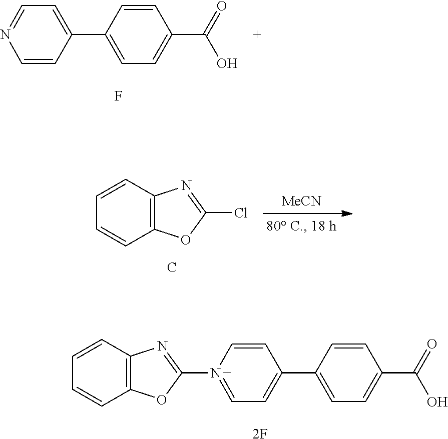

Compounds F (1.0 eq, 0.005 mol) and C (1.0 eq, 0.005 mol) were combined in a 20 mL microwave vial, followed by the addition of MeCN (8.0 mL). The reaction mixture was stirred at 80° C. for 18 h. The crude reaction mixture was purified by precipitation in diethyl ether. Recrystallization was performed for further purification. Briefly, the precipitated compound (50 mg) was dissolved in acetonitrile (1.0 mL) in a 20 mL microwave vial and heated at 80° C. for 30 min. Milli-Q water (2 mL) was added while the compound cooling down to room temperature. The crude reaction mixture was purified by precipitation using diethyl ether to give compound 2F as yellowish powder (1.47 g, 91%). ¹H NMR (500 MHz, MeOD) δ 9.92 (d, J=6.8 Hz, 2H), 8.81 (d, J=6.8 Hz, 2H), 8.33 (q, J=8.3 Hz, 5H), 7.99 (d, J=7.9 Hz, 1H), 7.94 (d, J=8.2 Hz, 1H), 7.70 (t, J=7.8 Hz, 1H), 7.65 (t, J=7.7 Hz, 1H). ¹³C NMR (125 MHz, MeOD) δ 166.94, 160.20, 152.66, 150.78, 141.06, 139.92, 137.04, 135.09, 130.72, 128.78, 127.29, 126.64, 125.13, 121.03, 111.35. LC/MS calculated for C₁₉H₁₃N₂O₃⁺=317.1; observed (M) 317.2.

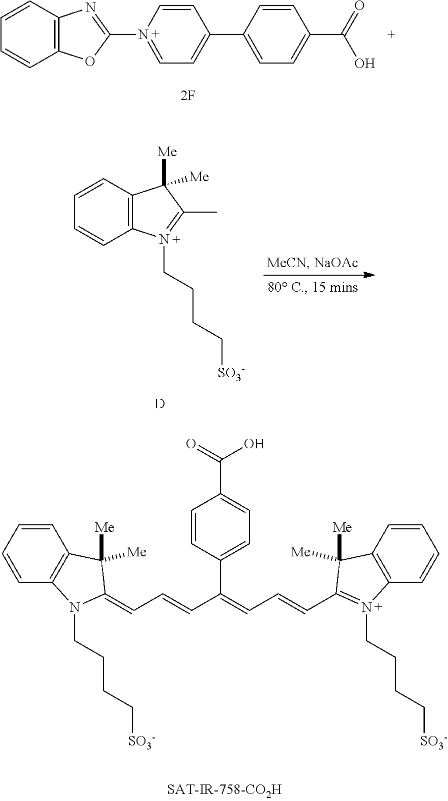

SAT-IR-758-CO₂H. Compounds 2F (1.0 eq, 0.63 mmol) and D (2.5 eq, 1.6 mmol) were combined in a 20 mL microwave vial, followed by the addition of acetonitrile (1.50 mL) and sodium acetate (2.5 eq, 1.6 mmol) The reaction mixture was stirred at 80° C. for 15 minutes. The crude reaction mixture was purified by using normal phase Combi Flash ISCO (0-40% DCM:MeOH) to give compound SAT-IR-758-CO2H as blue powder (350 mg, 72%). ¹H NMR (500 MHz, MeOD) δ 8.30 (d, J=7.6 Hz, 2H), 7.51 (d, J=7.7 Hz, 2H), 7.40 (s, 2H), 7.37 (s, 2H), 7.32 (d, J=8.1 Hz, 2H), 7.20 (t, J=7.5 Hz, 2H), 6.82 (s, 2H), 6.44 (s, 2H), 4.13 (s, 4H), 2.91 (t, J=6.8 Hz, 5H), 1.95 (d, J=6.1 Hz, 6H), 1.34 (s, 12H). ¹³C NMR (125 MHz, DMSO) δ 171.54, 148.73, 142.67, 140.94, 138.84, 129.77, 129.00, 128.72, 124.99, 122.79, 118.54, 111.63, 104.48, 67.49, 61.55, 58.45, 51.18, 49.06, 48.75, 44.12, 31.15, 27.68, 26.61, 25.59, 22.87, 18.36, 1.62. HRMS (ESI) calculated for C₄₆H₅₀N₃O₁₀S₂⁻=771.2779; observed (M⁻¹) 771.2773.

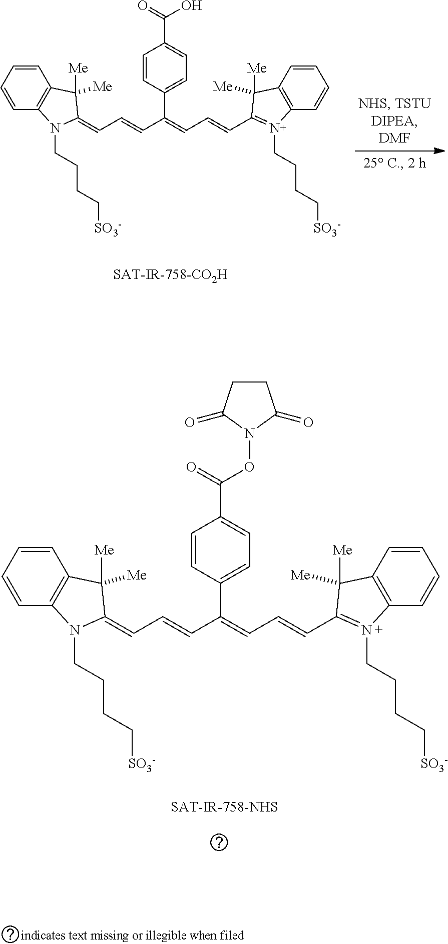

SAT-IR-758-NHS. SAT-IR-758-CO₂H (1.0 eq, 0.01 mmol) was added to DMF (0.50 mL) in a 20 mL microwave vial, followed by the addition of TSTU (2.0 eq, 0.02 mmol) and DIPEA (1.5 eq, 0.015 mmol). The reaction mixture was stirred at 25° C. for 0.5 hours followed by the addition of NHS (2.0 eq, 0.02 mmol). The reaction mixture was stirred at 25° C. for 2.0 h. The crude reaction mixture was purified by precipitating in diethyl ether. The compound was dried overnight in vacuum to give compound SAT-IR-758-NHS as blue powder (4 mg, 50%). FIRMS (ESI) calculated for C₄₆H₅₀N₃O₁₀S₂⁻, 868.2943; observed (M⁺²) 870.3081.

B. Synthesis of Cyclizing Cy7 (c-Cy7) and Spirocyclized Cy7 (Sc-Cy7)

Synthesis of c-Cy7(Cyclizing Cy7) and sc-Cy7 (spirocyclized Cy7). Certain embodiments are directed to fluorogenic probes based on cyclizing heptamethine cyanine (c-Cy7). These probes undergo a reversible cyclization process enabling them to toggle between a fluorescent lactone “on” state and a non-fluorescent zwitterionic “off” state, depending on the dielectric properties of the microenvironment. The non-fluorescent form readily permeates the phospolipid bilayer of the cell membrane due to the non-charged character of the probe. Once inside the cell, c-Cy7 interacts with its molecular targets, such as proteins or DNA, through linker-mediated binding. This interaction triggers a structural change that switches the probe to its fluorescent “on” state. The unique cyclization mechanism of c-Cy7 effectively minimizes background autofluorescence and provides a markedly enhanced signal-to-background ratio compared with conventional “always-on” Cy7 probes. This strategy overcomes the drawbacks of existing NIR dyes by offering controllable activation, reduced background interference, and enhanced versatility for clinical and preclinical applications.

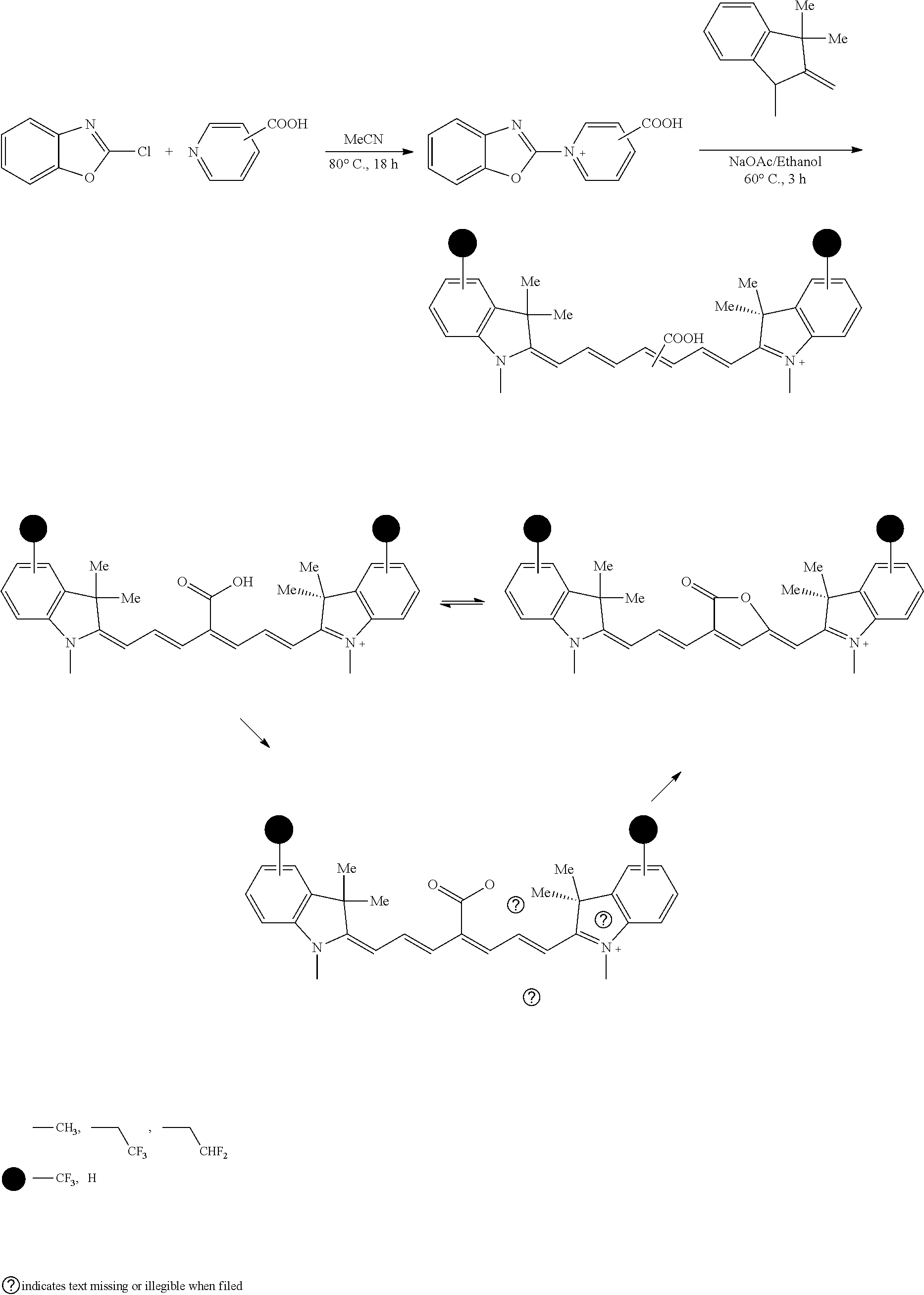

Fluorescent lactone “on” state(A) and non-fluorescent zwitterionic “off” state (C)

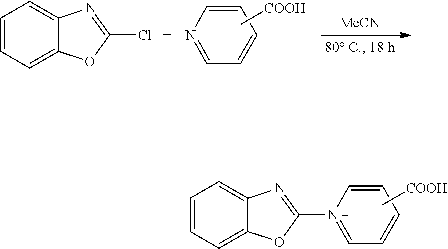

Compound A. Compound A (1.0 equiv, 8.1 mmol) and Compound B (1.0 equiv, 8.1 mmol) were dissolved in 10.0 mL of acetonitrile in a 20 mL microwave vial and heated in a sand bath at 100° C. for 18 h. The reaction progress was monitored by LC-MS. Upon completion, the product was precipitated using diethyl ether with a small amount of ethyl acetate, affording the desired compound as a solid (1.667 g, 85.1% yield).

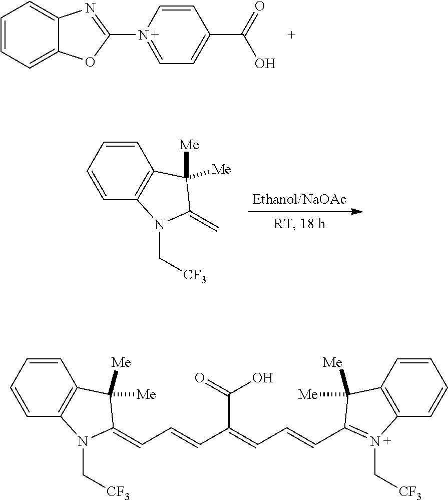

Compound IB. Compound 1A (1.0 equiv, 1.2 mmol), Compound B (3.0 equiv, 3.6 mmol), and sodium acetate (3.0 equiv, 3.6 mmol) were dissolved in 4.0 mL of ethanol in a 20 mL microwave vial and heated in a sand bath at 60° C. for 3 h. The reaction progress was monitored by LC-MS. Upon completion, the reaction mixture was purified by reverse-phase CombiFlash ISCO chromatography to afford Compound 1B as a dark-green powder after drying.

c-Cy 7 and sc-Cy7 scaffold with alternate substitutions.

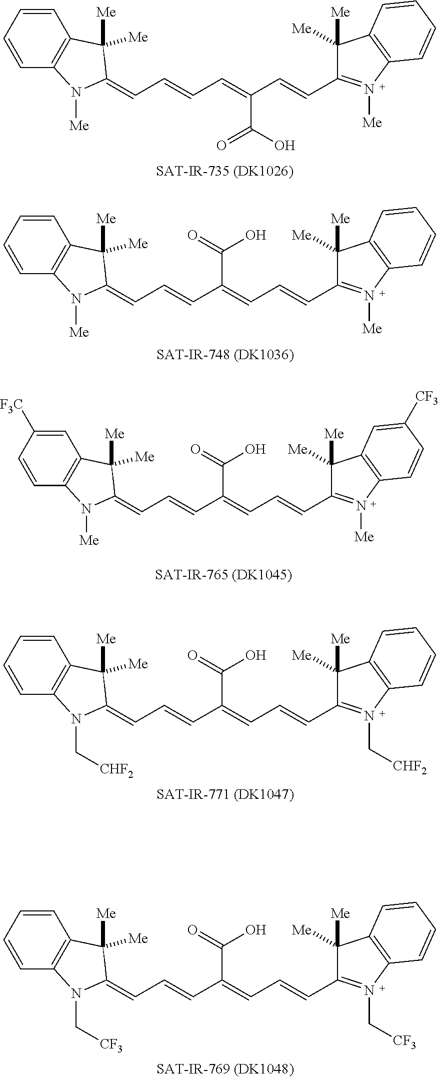

### Condition Optimization for the Synthesis of Cy7

| | | | Temperature | | |
|---|---|---|---|---|---|
| Cy7 | Solvent | Base | (° C.) | Time (h) | % yield |
| 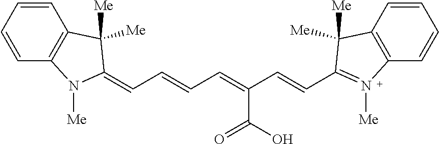 | MeCN | NaOAc | RT | 18 | 84.5 |
| 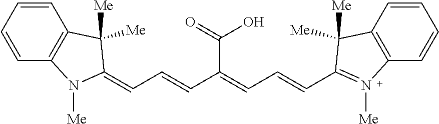 | EtOH | NaOAc | 60 | 3 | 67.7 |
| 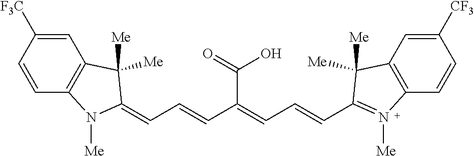 | EtOH | NaOAc | 60 | 3 | 33.3 |
| 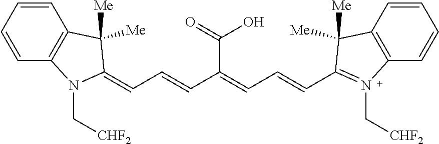 | EtOH | NaOAc | 60 | 3 | 96.5 |
| 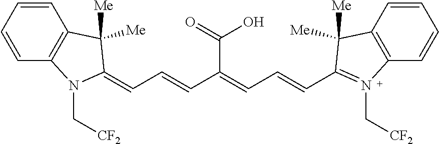 | EtOH | NaOAc | 60 | 3 | 49.1 |

### Photophysical Properties of Synthesized SAT-NIR-xx Dyes.

| | | λ_{max, abs} | λ_{max, em} | ε | Φ_{f} | Brightness |
|---|---|---|---|---|---|---|
| Compound | Solvent | (nm) | (nm) | (M⁻¹ cm⁻¹) | (%) | (ε × Φ_{f}) |
| DK 1026 | MeOH + 0.1% FA | 735 | 758 | 1.0 × 10⁵ | 21 | 21 × 10⁵ |
| SAT-IR-735 | 10% FBS | 752 | 765 | | 14 | |
| DK 1036 | MeOH + 0.1% FA | 748 | 774 | 0.9 × 10⁵ | 14 | 13 × 10⁵ |
| SAT-IR-748 | 10% FBS | 754 | 773 | | 9.2 | |
| DK 1045 | MeOH + 0.1% FA | 765 | 799 | 0.4 × 10⁵ | 11 | 4.4 × 10⁵ |
| SAT-IR-765 | 10% FBS | 755 | 772 | | 6.1 | |
| DK 1047 | MeOH + 0.1% FA | 771 | 806 | 0.6 × 10⁵ | 12 | 7.2 × 10⁵ |
| SAT-IR-771 | 10% FBS | 752 | 772 | | 4.5 | |
| DK 1048 | MeOH + 0.1% FA | 769 | 811 | 1.9 × 10⁵ | 7.9 | 15 × 10⁵ |
| SAT-IR-769 | 10% FBS | 748 | 770 | | 6.6 | |

### C. Biomedical Applications

The cyanine dyes produced by the method described herein are highly suitable for bioconjugation and in vivo imaging. The synthetic method's versatility allows for the incorporation of diverse functional groups across the polymethine chain, facilitating chemical conjugation to a range of biomolecules, including antibodies, peptides, nucleic acids, and other binding agents, for targeted imaging and detection in biological systems. These bioconjugation capabilities, combined with the method's scalability and use of commercially available reagents, make the dyes a significant advancement for fluorescence-guided surgery, molecular diagnostics, and biomedical research.

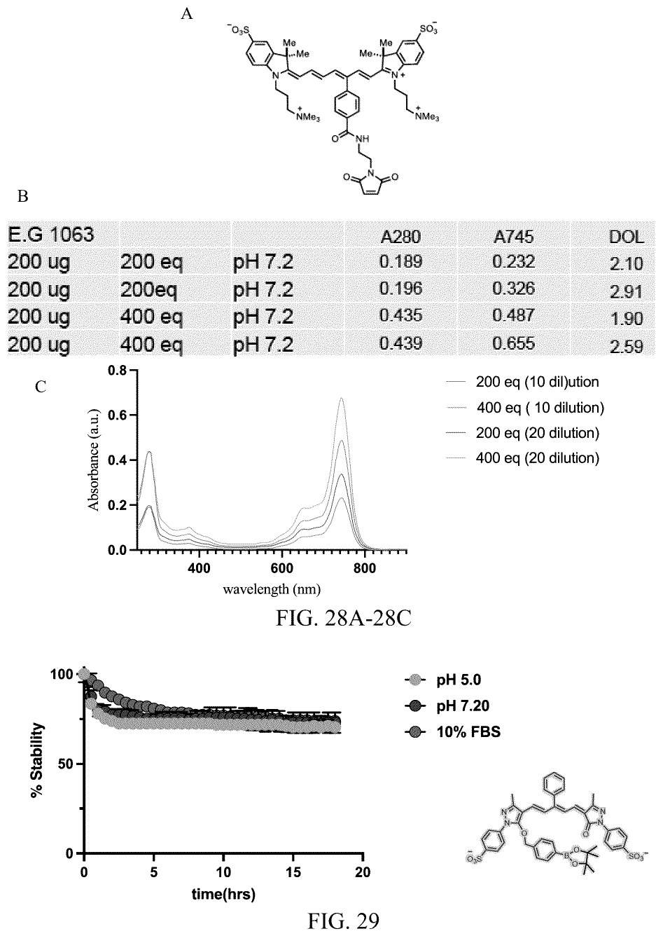

Antibody Conjugation for Targeted Imaging: The cyanine dyes can be conjugated to antibodies or other proteins to target specific biomarkers, such as tumor-associated antigens (e.g., HER2, EGFR, or PSA), for precise visualization in oncology applications. The dyes' modular scaffold allows for the introduction of reactive functional groups, such as carboxyl, amine, or thiol groups, enabling conjugation via standard chemistries like amide coupling (e.g., using EDC/NHS to link carboxyl-modified dyes to lysine residues), thiol-maleimide reactions (e.g., linking maleimide-modified dyes to cysteine residues) (FIG. 28), or click chemistry (e.g., azide-alkyne cycloaddition). These conjugation strategies ensure stable attachment without compromising antibody binding affinity, making the dyes ideal for fluorescence-guided surgery, where they enable real-time visualization of tumor margins to improve resection accuracy and reduce recurrence risk.

Beyond antibodies, the cyanine dyes can be conjugated to peptides, nucleic acids, aptamers, or small molecules to target specific receptors, enzymes, or cellular components. For example, peptides like RGD can be conjugated to cyanine dyes to target integrins in tumor vasculature, while nucleic acid conjugates can be used for fluorescence in situ hybridization (FISH) or RNA tracking in cells. Aptamers, with their high specificity for proteins or small molecules, can be linked to the dyes for diagnostic assays, and small molecules like folate can target folate receptor-positive cancers. The dyes' tunable functional groups support versatile conjugation chemistries, ensuring compatibility with diverse binding agents and applications.

In molecular diagnostics, cyanine dye-conjugated antibodies or other binding agents enable the detection of low-abundance biomarkers in tissue samples or bodily fluids, improving sensitivity for early disease detection (e.g., cancer or inflammatory markers). In biomedical research, these conjugates facilitate the visualization of cellular and molecular processes, such as protein localization, receptor dynamics, or gene expression, in vitro and in vivo. The dyes' stability under physiological conditions and resistance to aggregation ensure reliable performance when conjugated, addressing limitations of prior art dyes that suffer from fluorescence quenching or poor solubility.

The synthetic method is designed for scalability and industrial applicability, leveraging commercially available reagents to ensure cost-effectiveness and accessibility.

### II. OXONOL

Oxonols are anionic with oxygen-containing heterocycles, optimized for membrane potential sensing via Nernst-driven distribution, while cyanines are cationic or zwitterionic with nitrogen-based heterocycles (e.g., indole), offering tunable emission for multiplexing and protein labeling. Oxonols are less prone to aggregation but pH/ion-sensitive, whereas cyanines, historically aggregation-prone, are improved by β-substitution to disrupt π-π stacking. Oxonol dyes are primarily used to measure membrane potential in cells, permeating membranes based on potential gradients, with more negative potentials reducing dye accumulation. Their brightness and photostability make them ideal for microscopy, including live-cell imaging, where they serve as voltage-sensitive probes, detecting membrane potential changes through fluorescence intensity shifts.

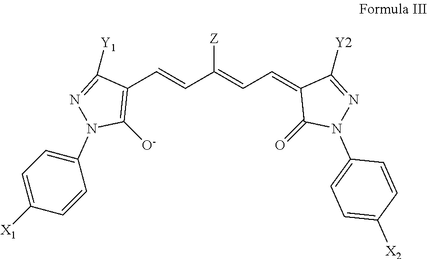

Certain embodiment include a compound of Formula III where X1 and X₂ are independently selected from hydrogen (H), SO₃, C1-C3 alkyl, C1-C3 sulfonate; Y₁ and Y₂ are independently selected from C1-C4 alkyl, SO₃, C1-C5 sulfonate, C3-C6 ethylene glycol, C1-C4 alkylamine, or choline; and Z is a carboxy, C1-C2 carboxamide, C1-C4 alkyl, C1-C4 alkoxy, carboxy, substituted phenylcarboxy (e.g., carboxyphenyl succinimide), substituted or unsubstituted alky, aryl (e.g., phenyl), or heteroatom moiety, the substituted alky, aryl (e.g., phenyl), or heteroatom moiety can have a substitution selected from halogen (e.g., fluoro, chloro, bromo, or iodo); C1-C6 alkyl, optionally substituted with one or more substituents selected from the group consisting of halogen, hydroxy, C1-C4 alkoxy, cyano, or amino; C2-C6 alkenyl, optionally substituted with one or more substituents selected from the group consisting of halogen, hydroxy, C1-C4 alkoxy, cyano, or amino; C2-C6 alkynyl, optionally substituted with one or more substituents selected from the group consisting of halogen, hydroxy, C1-C4 alkoxy, cyano, or amino; C1-C6 alkoxy, optionally substituted with one or more substituents selected from the group consisting of halogen, hydroxy, C1-C4 alkoxy, cyano, or amino; C1-C6 haloalkyl, including trifluoromethyl, difluoromethyl, or chloromethyl; C1-C6 haloalkoxy, including trifluoromethoxy or difluoromethoxy; hydroxy; cyano; nitro; amino, including —NH₂, —NH(C1-C4 alkyl), or —N(C1-C4 alkyl)₂; C1-C6 acyl, including acetyl or propionyl, optionally substituted with one or more substituents selected from the group consisting of halogen, hydroxy, or C1-C4 alkoxy; C1-C6 acyloxy, including acetoxy or propionyloxy; C1-C6 alkylsulfonyl, including methylsulfonyl or ethylsulfonyl; C1-C6 alkylsulfinyl, including methylsulfinyl or ethylsulfinyl; C1-C6 alkylthio, including methylthio or ethylthio; C6-C10 aryl, optionally substituted with one or more substituents selected from the group consisting of halogen, C1-C4 alkyl, C1-C4 alkoxy, hydroxy, cyano, nitro, or amino; C5-C10 heteroaryl, containing 1 to 4 heteroatoms independently selected from nitrogen, oxygen, or sulfur, optionally substituted with one or more substituents selected from the group consisting of halogen, C1-C4 alkyl, C1-C4 alkoxy, hydroxy, cyano, nitro, or amino; C3-C8 cycloalkyl, optionally substituted with one or more substituents selected from the group consisting of halogen, C1-C4 alkyl, C1-C4 alkoxy, hydroxy, cyano, or amino; C3-C8 heterocyclyl, containing 1 to 3 heteroatoms independently selected from nitrogen, oxygen, or sulfur, optionally substituted with one or more substituents selected from the group consisting of halogen, C1-C4 alkyl, C1-C4 alkoxy, hydroxy, cyano, or amino; Carboxyl, including —COOH or salts thereof, C1-C6 alkoxycarbonyl, including methoxycarbonyl or ethoxycarbonyl; Aminocarbonyl, including —C(O)NH₂, —C(O)NH(C1-C4 alkyl), or —C(O)N(C1-C4 alkyl)₂; Sulfonylamino, including —SO₂NH₂, —SO₂NH(C1-C4 alkyl), or —SO₂N(C1-C4 alkyl)₂; and combinations thereof, wherein two or more substituents on the phenyl moiety may optionally be taken together to form a fused C5-C6 cycloalkyl, C5-C6 heterocyclyl, C6-C10 aryl, or C5-C10 heteroaryl ring, each of which may be optionally substituted with one or more substituents selected from the group consisting of halogen, C1-C4 alkyl, C1-C4 alkoxy, hydroxy, cyano, nitro, or amino. In certain embodiments, the phenyl moiety is unsubstituted. In other embodiments, the phenyl moiety is substituted with 1 to 5 substituents, preferably 1 to 3 substituents, selected from the above list. Each substituent is independently selected, and where a substituent is itself substituted, the substitutions are as defined above unless otherwise specified.

One of the primary uses of oxonol dyes is in measuring membrane potentials in cells. They can permeate into cells based on the membrane potential, where more negative potentials inside the cell result in less dye accumulation. Due to their brightness and photostability, they are used in various forms of microscopy, including live-cell imaging. They function as voltage-sensitive probes, where changes in membrane potential can be visualized as changes in fluorescence intensity. Oxonols distribute across biological membranes according to the Nernst equation, which describes the equilibrium potential across a membrane. A change in membrane potential alters the distribution of the dye, affecting its fluorescence. They can respond quickly to changes in membrane potential, making them suitable for real-time monitoring. When used appropriately, they exhibit low toxicity to cells, allowing for long-term experiments. Limitations include photobleaching. Like many fluorescent dyes, oxonols can suffer from photobleaching, reducing their utility in prolonged imaging. Their fluorescence can be influenced by environmental factors like pH or ion concentration, which might require careful control of experimental conditions.

### A. Synthesis of Symmetrical Oxonol Dyes

The synthesis of oxonol dyes generally proceeds via pyridinium intermediates. Pyridinium intermediates are for by combining reactants in acetonitrile (MeCN) and heated with stirring, followed by precipitation of the product, solvent removal, and overnight vacuum drying, typically affording yields ranging from 61% to 97%. A pyridinium intermediate is reacted in the presence of sodium acetate (NaOAc) in 2 mL of ethanol (EtOH) and heated; triethylamine (Et₃N) is added, and the mixture is allowed to react for an additional time period. The crude product is then purified by normal-phase silica gel column using a 0-40% dichloromethane (DCM)/methanol (MeOH) gradient, with solvents removed by rotary evaporation and further drying in a Genevac evaporator, resulting in oxonol dye yields varying from 34% to 98% depending on the specific substituents.

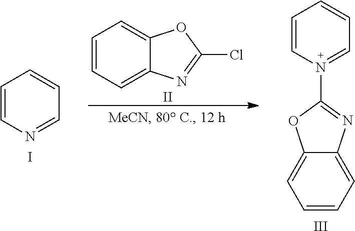

BK10029. Synthesis of pyridinium intermediate: (i) Add I and II in 2 mL of MeCN (Acetonitrile) heated at 80° C. stirred for 12 hours. (ii) Precipitate the product (III) in diethyl ether. (iii) Remove the solvent by centrifuge, vacuum dry the product overnight.

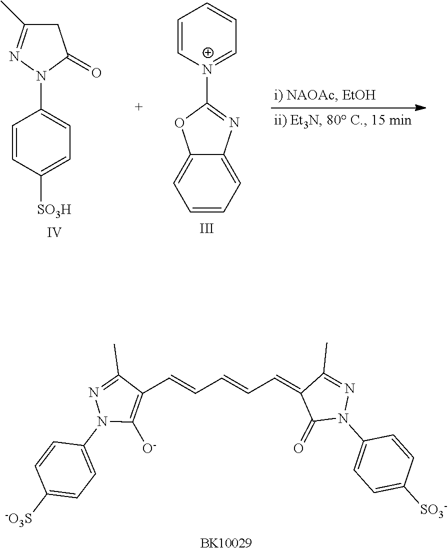

Add III and IV with NaOAc (Sodium Acetate) in 2 mL of EtOH (Ethanol) in a 20 mL reaction vial heated at 80° C. Then add Et₃N (Triethylamine) after 5 mins and let it react for another 10 mins with a typical % yield of BK10029=98%.

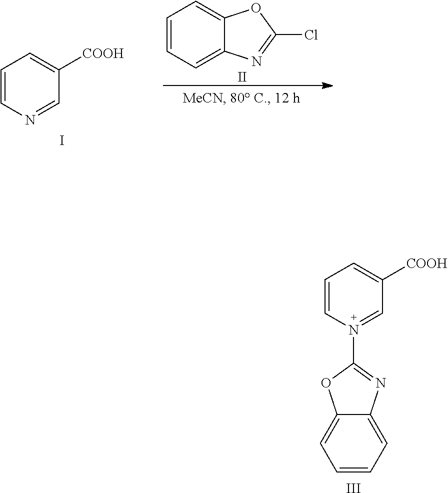

BK10014. Synthesis of pyridinium intermediate (i) Add I and II in 2 mL of MeCN (Acetonitrile) heated at 80° C. stirred for 12 hours. (ii) Precipitate the product (III) in diethyl ether. (iii) Remove the solvent by centrifuge, vacuum dry the product overnight. (iv) % yield=77%.

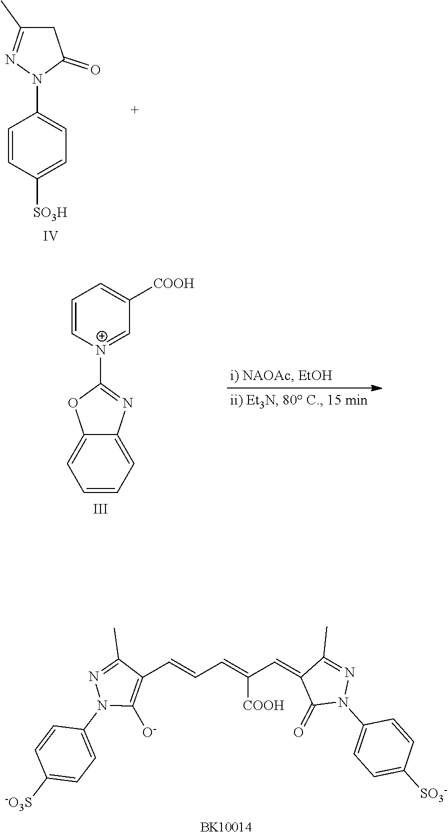

Synthesis of oxonol dye: (i) Add III and IV with NaOAc (Sodium Acetate) in 2 mL of EtOH (Ethanol) in a 20 mL reaction vial heated at 80° C. (ii) Then add Et₃N (Triethylamine) after 5 mins and let it react for another 10 mins. (iii) Purify using dry loading ISCO Normal Phase 0-40% DCM/MeOH (Dichloromethane/Methanol) in Silica 12 g Column. (iv) Remove solvents by rotavap, further dry in Genevac. (v) % yield of BK10014=36%

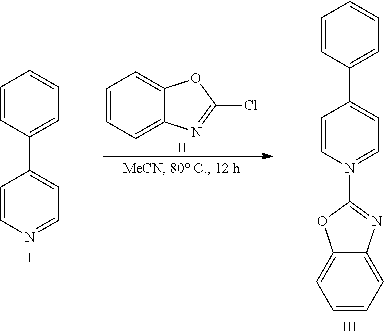

BK10015. Synthesis of pyridinium intermediate (i) Add I and II in 2 mL of MeCN (Acetonitrile) heated at 80° C. stirred for 12 hours. (ii) Precipitate the product (III) in diethyl ether. (iii) Remove the solvent by centrifuge, vacuum dry the product overnight. (iv) % yield=82%

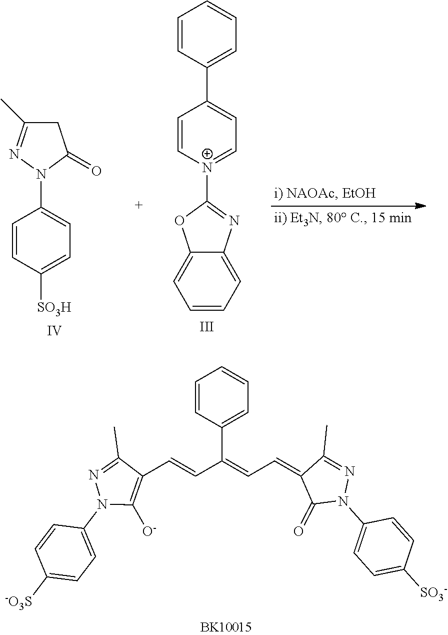

Synthesis of oxonol dye: (i) Add III and IV with NaOAc (Sodium Acetate) in 2 mL of EtOH (Ethanol) in a 20 mL reaction vial heated at 80° C. (ii) Then add Et₃N (Triethylamine) after 5 mins and let it react for another 10 mins. (iii) Purify using dry loading ISCO Normal Phase 0-40% DCM/MeOH (Dichloromethane/Methanol) in Silica 12 g Column. (iv) Remove solvents by rotavap, further dry in Genevac. (v) % yield of BK10015=82.5%

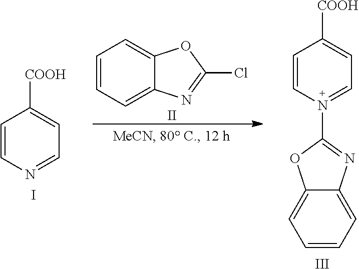

BK10016. Synthesis of pyridinium intermediate (i) Add I and II in 2 mL of MeCN (Acetonitrile) heated at 80° C. stirred for 12 hours. (ii) Precipitate the product (III) in diethyl ether. (iii) Remove the solvent by centrifuge, vacuum dry the product overnight. (iv) % yield=96.7%

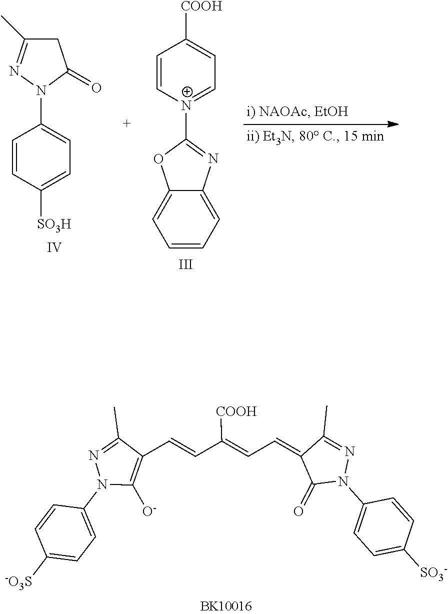

Synthesis of oxonol dye (i) Add III and IV with NaOAc (Sodium Acetate) in 2 mL of EtOH (Ethanol) in a 20 mL reaction vial heated at 80° C. (ii) Then add Et₃N (Triethylamine) after 5 mins and let it react for another 10 mins. (iii) Purify using dry loading ISCO Normal Phase 0-40% DCM/MeOH (Dichloromethane/Methanol) in Silica 12 g Column. (iv) Remove solvents by rotavap, further dry in Genevac. (v) % yield of BK10016=34%

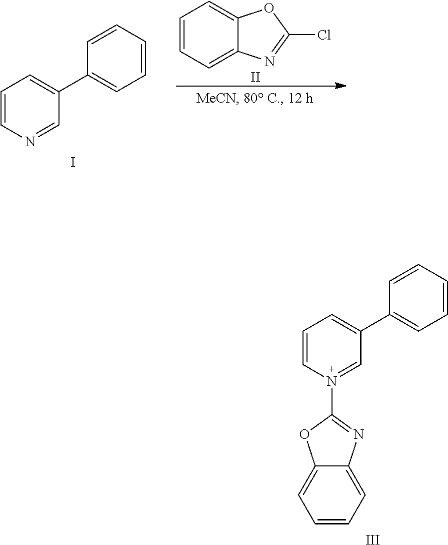

BK10018. Synthesis of pyridinium intermediate (i) Add I and II in 2 mL of MeCN (Acetonitrile) heated at 80° C. stirred for 12 hours. (ii) Precipitate the product (III) in diethyl ether. (iii) Remove the solvent by centrifuge, vacuum dry the product overnight. (iv) % yield=61.4%

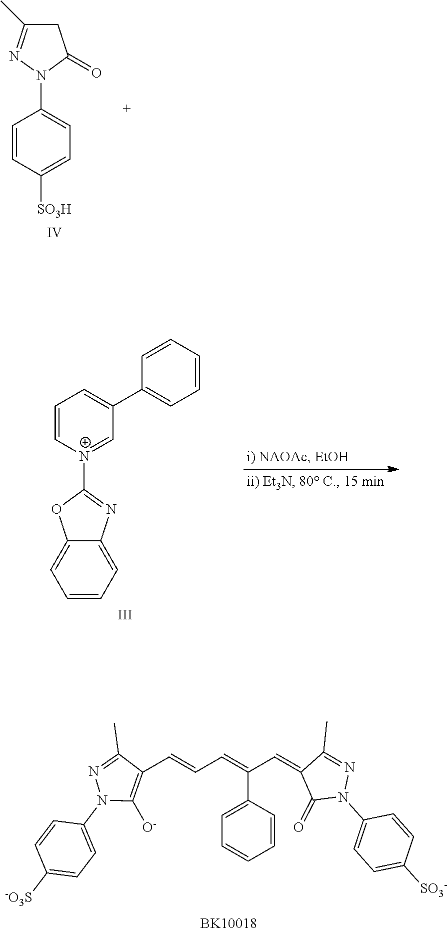

Synthesis of oxonol dye (i) Add III and IV with NaOAc (Sodium Acetate) in 2 mL of EtOH (Ethanol) in a 20 mL reaction vial heated at 80° C. (ii) Then add Et₃N (Triethylamine) after 5 mins and let it react for another 10 mins. (iii) Purify using dry loading ISCO Normal Phase 0-40% DCM/MeOH (Dichloromethane/Methanol) in Silica 12 g Column. (iv) Remove solvents by rotavap, further dry in Genevac. (v) % yield of BK10018=62%

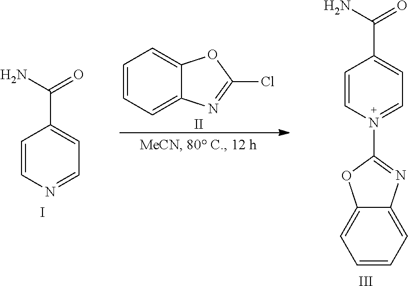

BK10020. Synthesis of pyridinium intermediate (i) Add I and II in 2 mL of MeCN (Acetonitrile) heated at 80° C. stirred for 12 hours. (ii) Precipitate the product (III) in diethyl ether. (iii) Remove the solvent by centrifuge, vacuum dry the product overnight. (iv) % yield=79.4%.

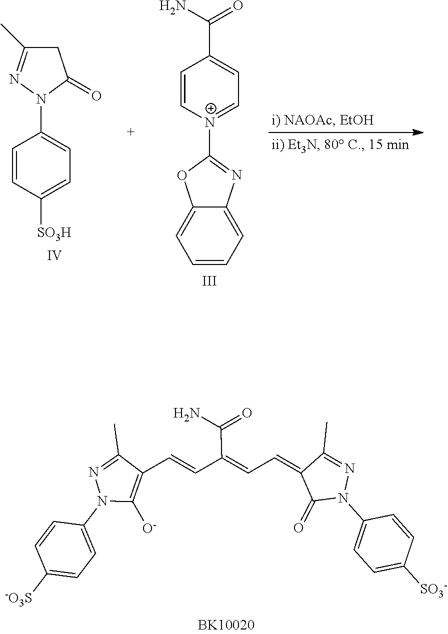

Synthesis of oxonol dye (i) Add III and IV with NaOAc (Sodium Acetate) in 2 mL of EtOH (Ethanol) in a 20 mL reaction vial heated at 80° C. (ii) Then add Et₃N (Triethylamine) after 5 mins and let it react for another 10 mins. (iii) Purify using dry loading ISCO Normal Phase 0-40% DCM/MeOH (Dichloromethane/Methanol) in Silica 12 g Column. (iv) Remove solvents by rotavap, further dry in Genevac. (v) % yield of BK10029=51.6%.

B. Synthesis of Symmetrical Oxonol Dyes with Diverse Pyrazoles

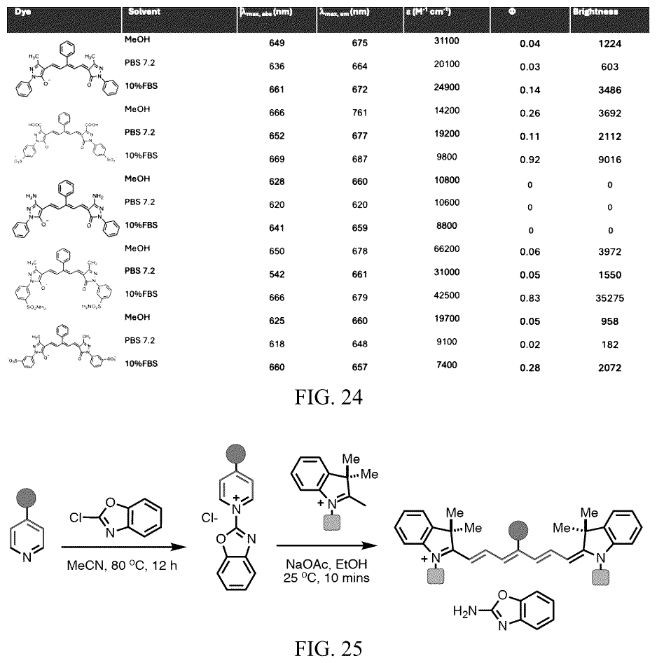

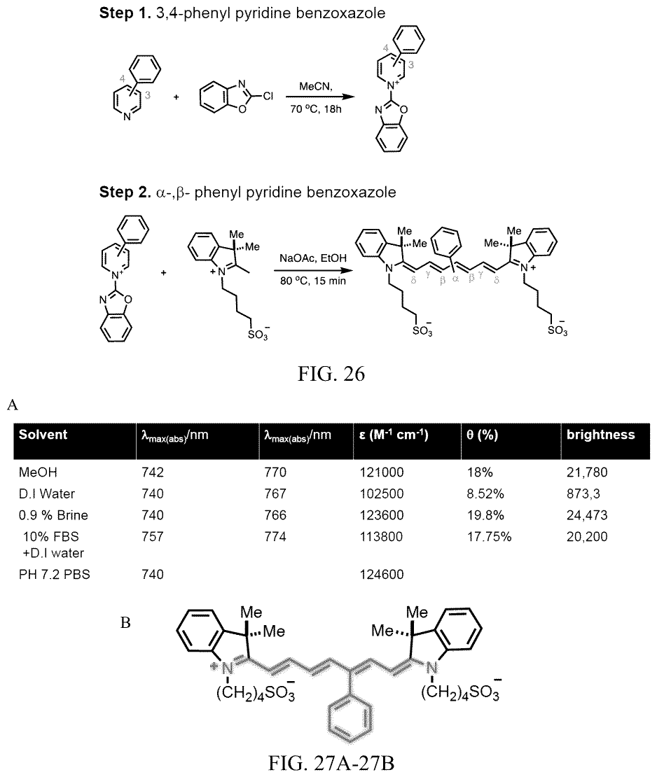

Synthesis of oxonol dye. (i) Add III and IV with NaOAc (Sodium Acetate) in 2 mL of EtOH (Ethanol) in a 20 mL reaction vial heated at 80° C. (ii) Then add Et₃N (Triethylamine) after 5 mins and let it react for another 10 mins. (iii) Purify using dry loading ISCO Normal Phase 0-40% DCM/MeOH (Dichloromethane/Methanol) in Silica 12 g Column. (iv) Remove solvents by rotavap, further dry in Genevac. Photophysical properties can be found in FIG. 24.

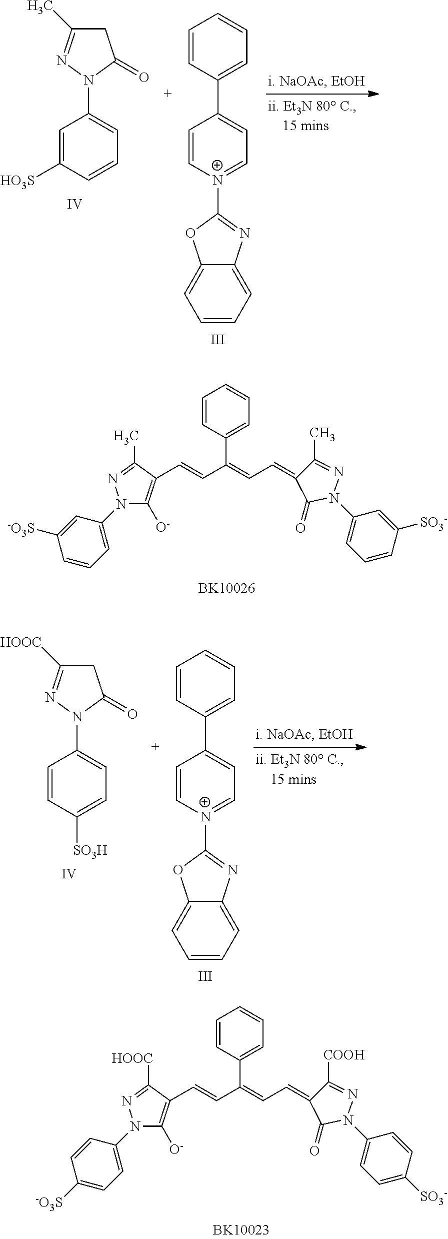

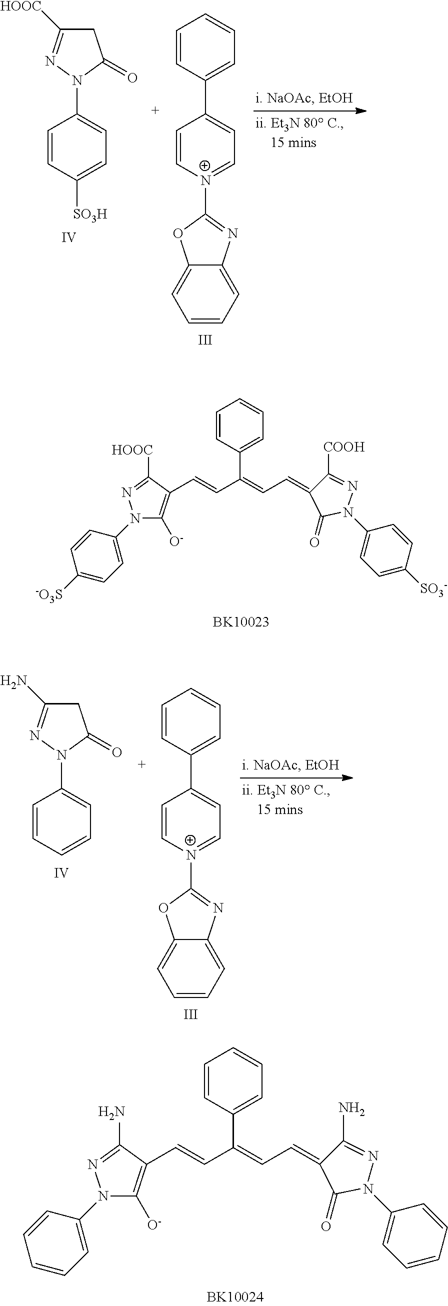

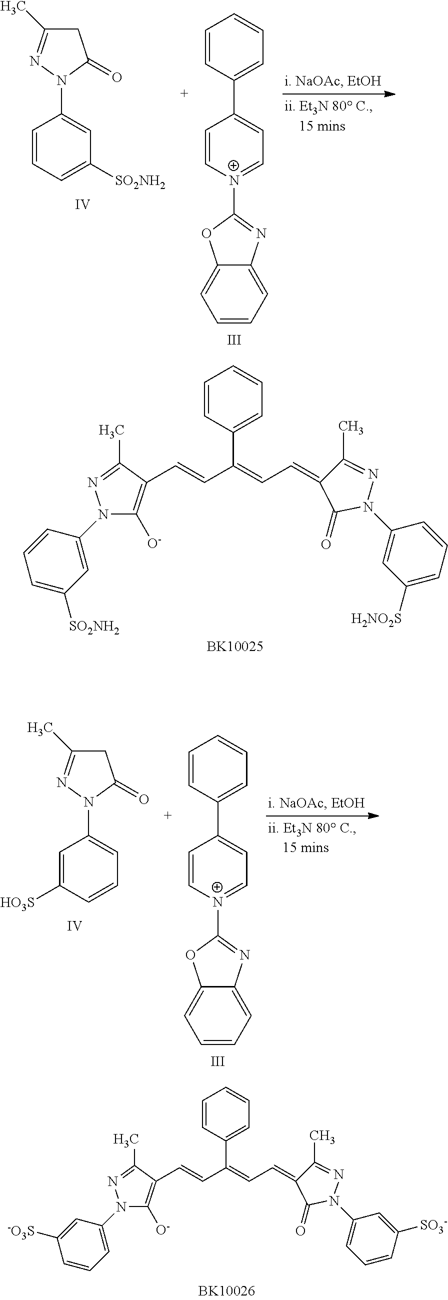

### III. DIVERSIFIED OXONOL FLUOROPHORES FOR BIOCONJUGATION

Synthesis of pyridinium intermediate (i) Add I and II in 2 mL of MeCN (Acetonitrile) heated at 80° C. stirred for 12 hours. (ii) Precipitate the product in diethyl ether. (iii) Centrifuge, remove the solvent and dry the product overnight.

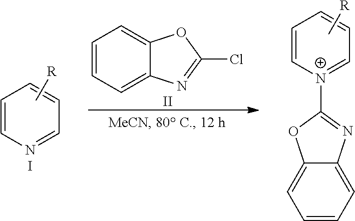

Synthesis of oxonol dye (i) Add I and II with NaOAc (Sodium Acetate) in 2 mL of EtOH (Ethanol) in a 20 mL scintillation vial heated at 80° C. (ii) Then add Et₃N (Triethylamine) after 5 mins and let it react for another 10 mins. (iii) Purify using dry loading ISCO Normal Phase 0-40% DCM/MeOH (Dichloromethane/Methanol). (iv) Remove solvents by rotavap, further dry in Genevac.

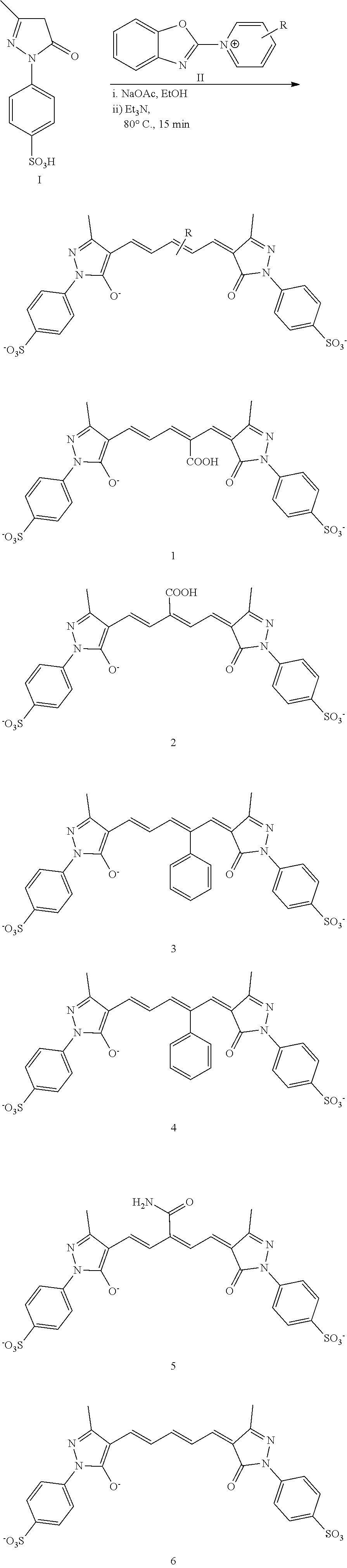

### IV. SYNTHESIS OF FLUOROGENIC PROBE

Fluorogenic probes, non-fluorescent until activated by specific chemical or biological events, enhance oxonol dye utility in sensing and imaging. Conjugating oxonols with fluorogenic moieties, such as enzyme-cleavable or pH-sensitive groups, improves specificity and sensitivity. For example, a fluorogenic moiety activated by enzymatic cleavage or pH changes enables targeted detection of cellular processes while preserving oxonol voltage-sensing capabilities. This approach reduces background fluorescence, enhances signal-to-noise ratios, and supports real-time, selective imaging in complex biological systems.

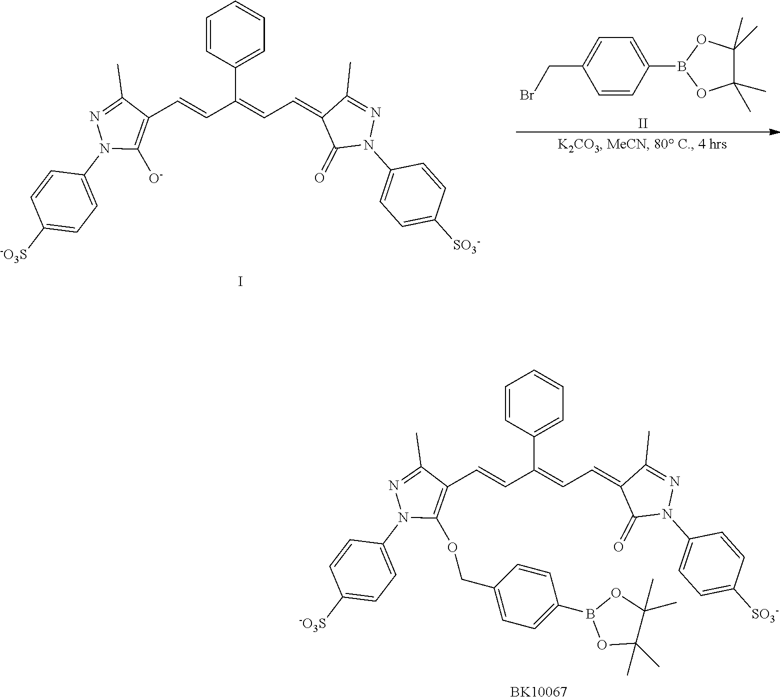

Add I & II with K₂CO₃ in MeCN for 18 hrs @80° C. Dye formed and confirmed with LCMS. Remove vial, dry with N₂. Purify with Dry loading ISCO reverse phase 0-95% B Water/MeCN. Genevac drying. Test Serum Stability for 18 hrs FIG. 29.

### V. BRIGHTER AND PHOTOSTABLE FLUOROPHORES

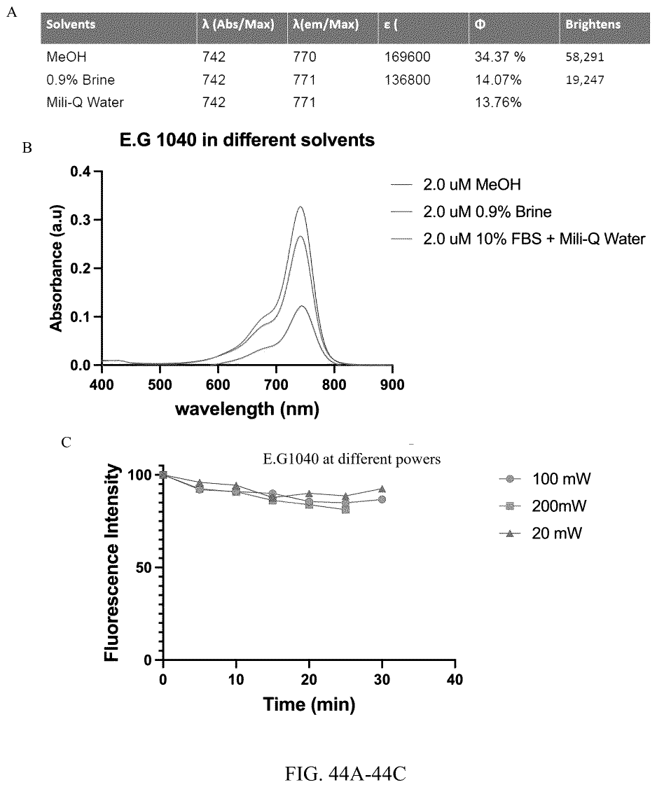

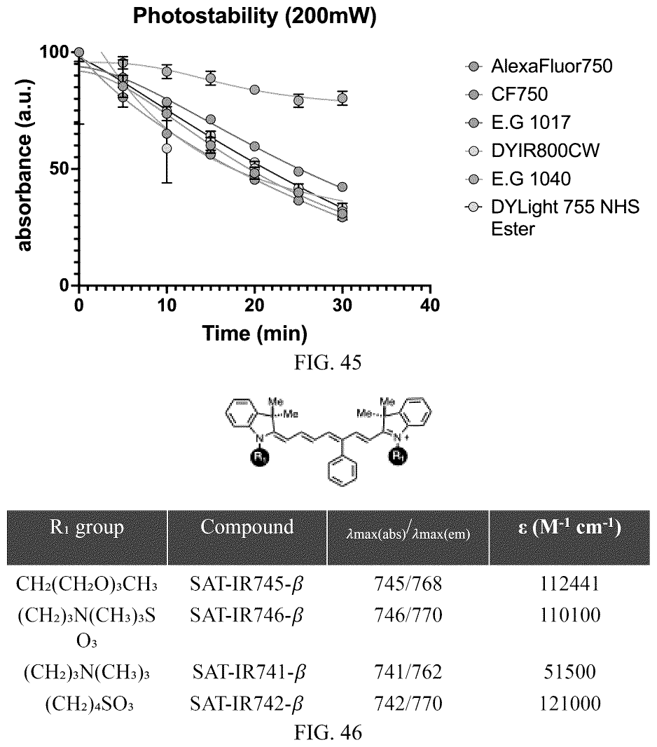

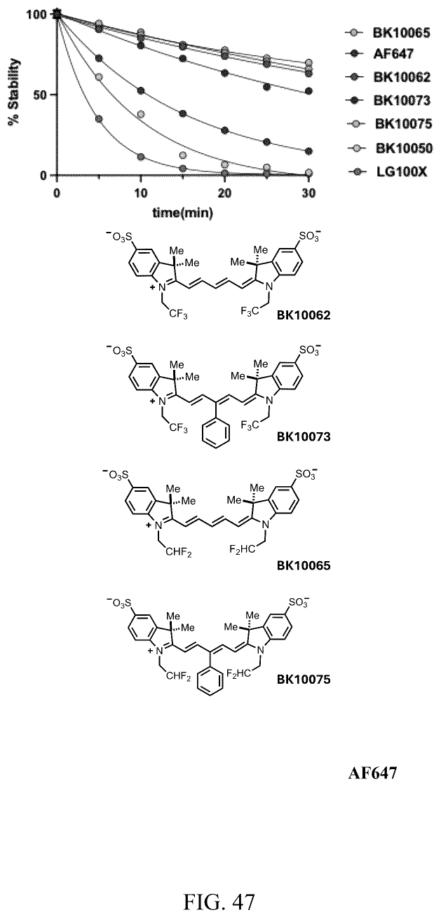

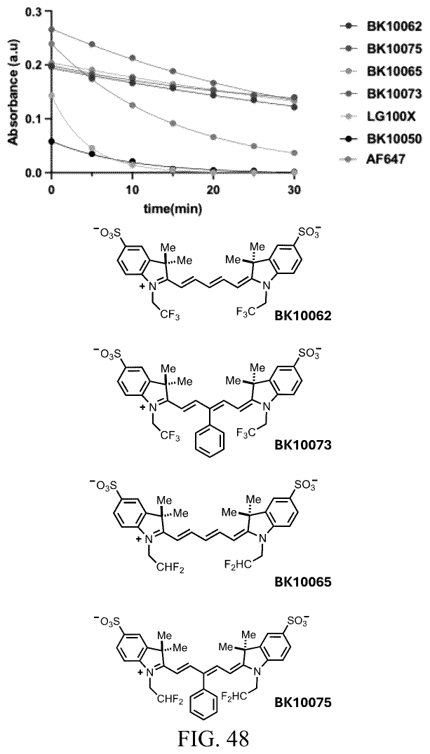

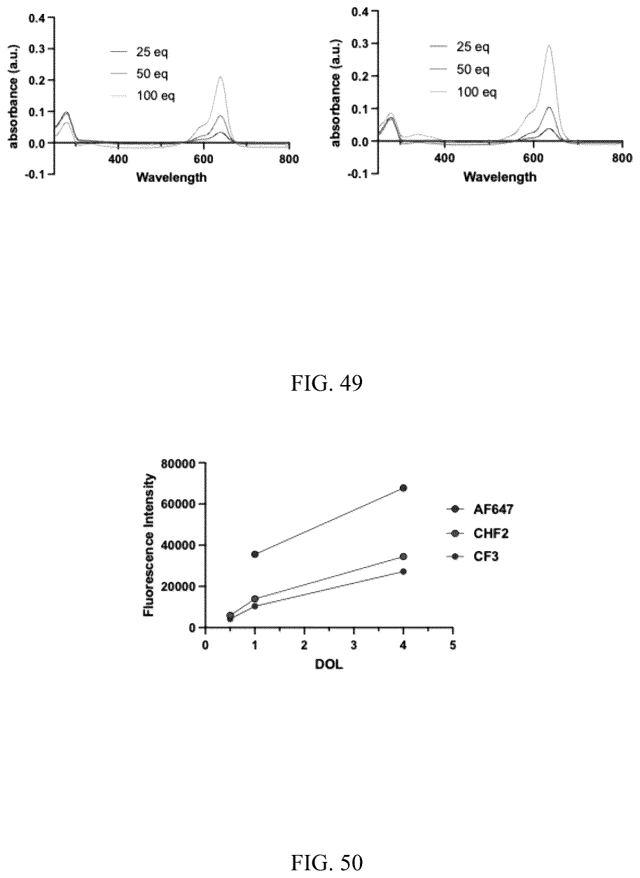

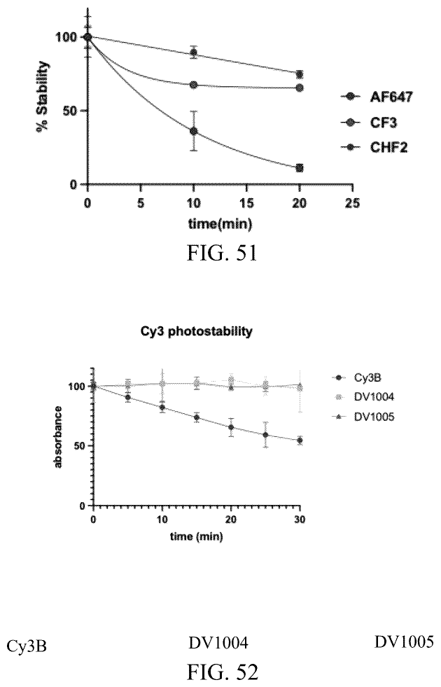

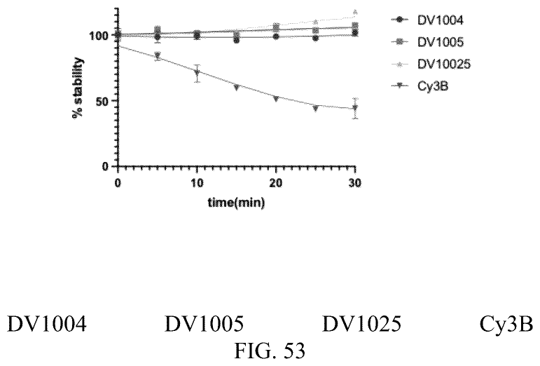

In certain embodiments, modifications enhance fluorophore photostability. Stability data for E.G.1040 in various solvents are presented in FIG. 44A-44C. Photostability assays comparing E.G.1040, DYIR800CW, and E.G.1070 are shown in FIG. 45.

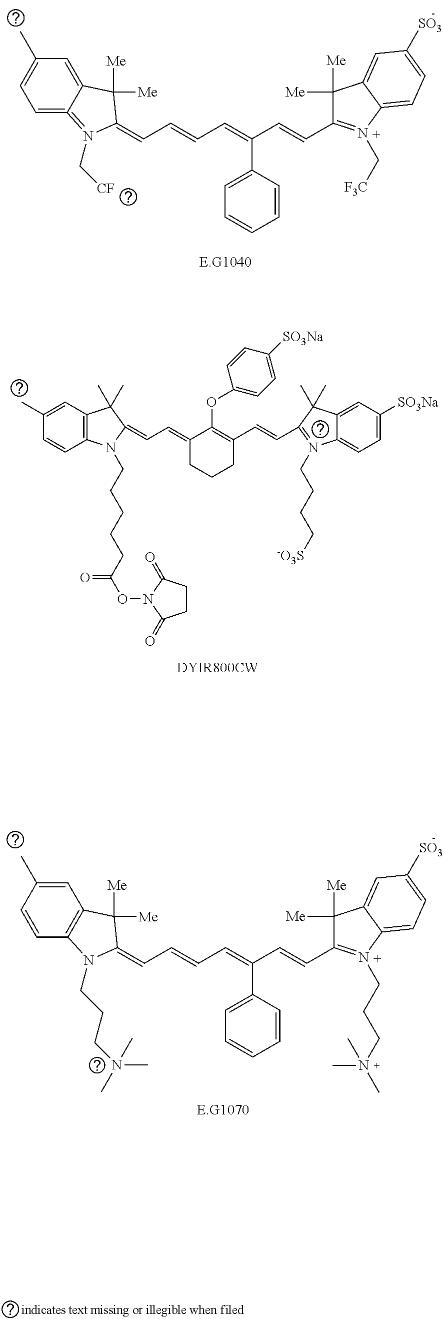

### VI. CHEMICAL DEFINITIONS

Various chemical definitions related to such compounds are provided as follows.

As used herein, the term “nitro” means —NO₂; the term “halo” designates —F, —Cl, —Br or —I; the term “mercapto” means —SH; the term “cyano” means —CN; the term “azido” means —N₃; the term “silyl” means —SiH₃, and the term “hydroxy” means —OH.

The term “alkyl,” by itself or as part of another substituent, means, unless otherwise stated, a linear (i.e. unbranched) or branched carbon chain, which may be fully saturated, mono- or polyunsaturated. An unsaturated alkyl group is one having one or more double bonds or triple bonds. Saturated alkyl groups include those having one or more carbon-carbon double bonds (alkenyl) and those having one or more carbon-carbon triple bonds (alkynyl). The groups, —CH₃ (Me), —CH₂CH₃ (Et), —CH₂CH₂CH₃ (n-Pr), —CH(CH₃)₂(iso-Pr), —CH₂CH₂CH₂CH₃ (n-Bu), —CH(CH₃)CH₂CH₃ (sec-butyl), —CH₂CH(CH₃)₂(iso-butyl), —C(CH₃)₃(tert-butyl), —CH₂C(CH₃)₃ (neo-pentyl), are all non-limiting examples of alkyl groups.

The term “heteroalkyl,” by itself or in combination with another term, means, unless otherwise stated, a linear or branched chain having at least one carbon atom and at least one heteroatom selected from the group consisting of O, N, S, P, and Si. In certain embodiments, the heteroatoms are selected from the group consisting of O and N. The heteroatom(s) may be placed at any interior position of the heteroalkyl group or at the position at which the alkyl group is attached to the remainder of the molecule. Up to two heteroatoms may be consecutive. The following groups are all non-limiting examples of heteroalkyl groups: trifluoromethyl, —CH₂F, —CH₂ Cl, —CH₂ Br, —CH₂ OH, —CH₂ OCH₃, —CH₂ OCH₂ CF₃, —CH₂OC(O)CH₃, —CH₂ NH₂, —CH₂ NHCH₃, —CH₂N(CH₃)₂, —CH₂CH₂Cl, —CH₂CH₂OH, CH₂CH₂OC(O)CH₃, —CH₂CH₂NHCO₂C(CH₃)₃, and —CH₂Si(CH₃)₃.

The terms “cycloalkyl” and “heterocyclyl,” by themselves or in combination with other terms, means cyclic versions of “alkyl” and “heteroalkyl”, respectively. Additionally, for heterocyclyl, a heteroatom can occupy the position at which the heterocycle is attached to the remainder of the molecule.

The term “aryl” means a polyunsaturated, aromatic, hydrocarbon substituent. Aryl groups can be monocyclic or polycyclic (e.g., 2 to 3 rings that are fused together or linked covalently). The term “heteroaryl” refers to an aryl group that contains one to four heteroatoms selected from N, O, and S. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

Various groups are described herein as substituted or unsubstituted (i.e., optionally substituted). Optionally substituted groups may include one or more substituents independently selected from: halogen, nitro, cyano, hydroxy, amino, mercapto, formyl, carboxy, oxo, carbamoyl, substituted or unsubstituted alkyl, substituted or unsubstituted heteroalkyl, alkoxy, alkylthio, alkylamino, (alkyl)₂amino, alkylsulfinyl, alkylsulfonyl, arylsulfonyl, substituted or unsubstituted cycloalkyl, substituted or unsubstituted heterocyclyl, substituted or unsubstituted aryl, and substituted or unsubstituted heteroaryl. In certain aspects the optional substituents may be further substituted with one or more substituents independently selected from: halogen, nitro, cyano, hydroxy, amino, mercapto, formyl, carboxy, carbamoyl, unsubstituted alkyl, unsubstituted heteroalkyl, alkoxy, alkylthio, alkylamino, (alkyl)₂amino, alkylsulfinyl, alkylsulfonyl, arylsulfonyl, unsubstituted cycloalkyl, unsubstituted heterocyclyl, unsubstituted aryl, or unsubstituted heteroaryl. Exemplary optional substituents include, but are not limited to: —OH, oxo (═O), —Cl, —F, Br, C₁₋₄alkyl, phenyl, benzyl, —NH₂, —NH(C₁₋₄alkyl), —N(C₁₋₄alkyl)₂, —NO₂, —S(C₁₋₄alkyl), —SO₂(C₁₋₄alkyl), —CO₂(C₁₋₄alkyl), and —O(C₁₋₄alkyl).

The term “alkoxy” means a group having the structure —OR′, where R′ is an optionally substituted alkyl or cycloalkyl group. The term “heteroalkoxy” similarly means a group having the structure —OR, where R is a heteroalkyl or heterocyclyl.

The term “amino” means a group having the structure —NR′R″, where R′ and R″ are independently hydrogen or an optionally substituted alkyl, heteroalkyl, cycloalkyl, or heterocyclyl group. The term “amino” includes primary, secondary, and tertiary amines.

The term “oxo” as used herein means an oxygen that is double bonded to a carbon atom.

The term “alkylsulfonyl” as used herein means a moiety having the formula —S(O₂)—R′, where R′ is an alkyl group. R′ may have a specified number of carbons (e.g. “C₁₋₄ alkylsulfonyl”)

### VII. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention and thus can be considered to constitute preferred modes for its practice. However, those of skill in the art should appreciate that many changes can be made in the specific embodiments which are disclosed and still obtain a like or similar result without departing from the spirit and scope of the invention.

### Example 1 Diversified Oxonol Fluorophores for Bioconjugation

### Methods—General Procedure

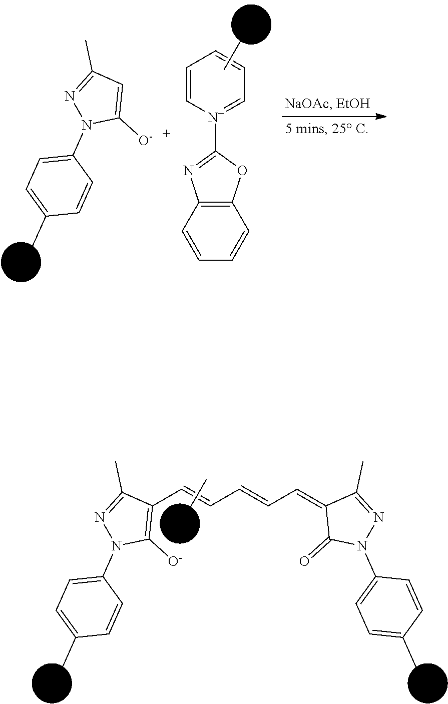

### Results—Examples of Structures

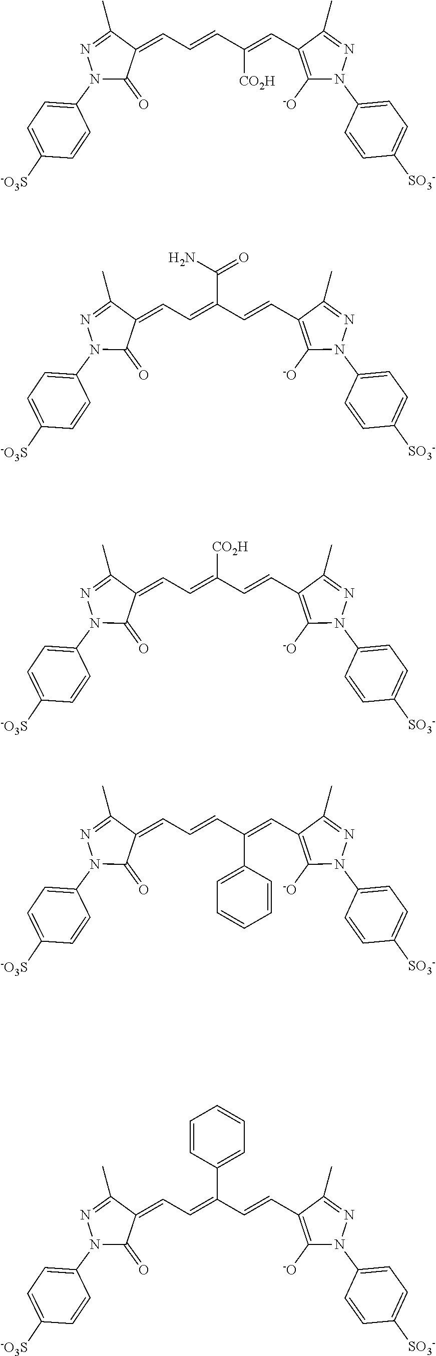

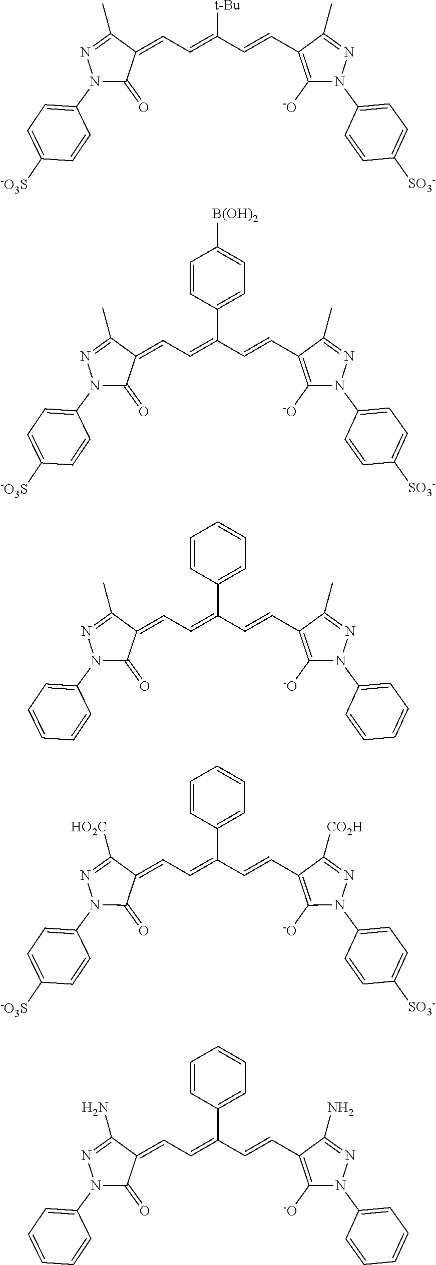

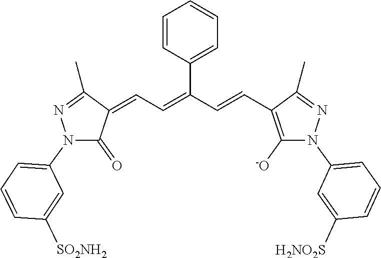

### Example 2: Unsymmetrical Heptamethine Cyanines Prevent Aggregation

### A. Methods

General Experimental Procedures. Reagents and solvents were purchased from Sigma-Aldrich, Ambeed, Thermoscientific, BTC, TCI, VWR, fisher chemical, and CIL and used without further purification unless otherwise stated. 1H and 13C NMR spectra were recorded on a Bruker 300 and 500 NMR spectrometer. Chemical shifts are presented in ppm and referenced by residual solvent peak and are reported relative to deuterated solvent signals (CDCl3: 1H NMR=7.24, 13C NMR=77.0, MeOD: 1H NMR=3.30, 13C NMR=49.0, DMSO-d6: 1H NMR=2.50, 13C NMR=39.5). The following abbreviations were used to explain the multiplicities: s=singlet, d=doublet, t=triplet, q=quartet, quint=quintet, dd=double doublet, dt=double triplet, dq=double quartet, m=multiplet. Combi Flash Next Gen 300+Teledyne Normal phase and Reverse phase ISCO Flash column chromatography purification was performed. LC/MS was performed using an Agilent 1260 Infinity LC/MS was equipped with 1260 Vial sampler, 1260 Binary Pump, G1322A Degasser. The LC/MS runs had a gradient of 5-95% MeCN/H₂O with 0.05% formic acid over 10 min at a flow rate of 0.5 mL/min. Agilent Preparatory HPLC was equipped with 1290 and 1260 Infinity II Preparative Binary Pump equipped with Diode Array and Multiple Wavelength Detector. The HPLC was installed with an Agilent capillary TS column (1×250 XS), and each run employed a gradient of 5-95% MeCN/H₂O with 0.1% TFA over 10 min at a flow rate of 10 mL/min. All statistical analyses were carried out by Graphpad Prism version 9.0 (Graphpad Software).

Absorbance Measurements. Absorbance spectra were obtained on JASCO V-770 spectrophotometer to form plots of absorbance vs. Concentration (0.5-20 μM). The absorbance spectra were measured from 400-900 nm (1 nm step size). Spectra were recorded with synthetic quartz cuvette (ThorLabs) holder with 10 mm path length.

Extinction coefficients were determined in Methanol, 10% FBS+Mili-Q Water, 0.9% Brine, and Mili-Q Water (0.1-5 μM). Spectra were recorded with a disposable micro-UV-Cuvette holder with 10 mm path length, with absorbance at the highest concentration ≤0.20. Three independent readings were taken at each concentration.

Fluorescence measurements. Fluorescence spectra were measured using FluoroMax from Horiba scientific using with 2.5 nm excitation and emission slit widths, and a 0.1 s integration rate. The fluorescence was measured in methanol, 0.9% Brine, Mili-Q Water and in 10% FBS+Mili-Q Water. Probe was excited at 700 nm, and emission was collected from 730-900 nm. Measurements were carried out a concentration with optical density of less than 0.1 in solvent. Spectra were recorded with synthetic quartz cuvette (ThorLabs) holder with 10 mm path length.

Fluorescent Quantum Yields. Fluorescent quantum yield was determined using relative quantum yield calculations. Quantum yield was determined relative to common fluorescent standards (ICG Ff=0.13 in DMSO) for optically dilute solutions (absorbance in the range from 0.01-0.05). The parameters used to gather the quantum yields were: excitation at 700 nm, emission to 730-900 nm, and a slit width of 2.5 nm. Spectra were recorded with synthetic quartz cuvette (ThorLabs) holder with 10 mm path length.

The fluorescence quantum yield was calculated using the following formula:

$${ϕ}_{S} = {ϕ}_{R} \times \frac{{I}_{s}}{{I}_{R}} \times \frac{{A}_{r}}{{A}_{s}} \times ( \frac{{n}_{s}^{2}}{{n}_{R}^{2}} )$$

where φₛ=Quantum yield of the sample; φᵣ=Quantum yield of reference; Iₛ=Integrated fluorescence area of the sample; Iᵣ=Integrated fluorescence area of the reference; Aᵣ=Absorbance of the reference at the excitation wavelength; Aₛ=Absorbance of the sample at the excitation wavelength; ηₛ=Refractive index of the sample; ηᵣ=Refractive index of the reference

Antibody Conjugation and DOL Determination. 0.2 mg of 30 mg/mL of IgG and of SAT-IR-746-NHS, SAT-IR-758-NHS, and IRDye800 CW-NHS (10 mM stock in DMSO; 20-50 eq) was added to 190 μL of PBS (pH 7.2) in 1.5 mL Eppendorf tube. The mixture was covered in aluminum foil and stirred for 2 h on orbital mini shaker at room temperature. The reaction mixture was eluted through a pH 7.2 PBS equilibrated Zeba spin DS column (7K MWCO, Thermo Fisher Scientific) to remove unreacted free dye. This was followed by buffer exchange and further purification using slide-A-Lyzer MINI Dialysis Devices (10K MWCO, 0.5 mL, Thermofisher) in PBS (1×, pH 7.2). The buffer was replaced twice, at 1 and 3 h time interval. Purified conjugate was collected in 0.5 mL Eppendorf. The antibody conjugates were concentrated using Amicon® Ultra Centrifugal Filter Devices (10 k MWCO; Milipore Sigma) by centrifuging at 10,000 rpm for 10 mins 3 times. The degree of labeling (DOL; dye to antibody ratio) was determined by measuring absorption at 280 nm and at each dyes maximum absorbance. A correction factor of 0.03 at 280 nm was used in the calculations. DOL and protein concentrations were measured by the following equations. The antibody conjugates were stored at 4° C. No changes in absorbance spectra were monitored over 28 days.

DOL determination. The absorbance spectra of antibody-fluorophore conjugates were collected in PBS buffer (pH 7.2, 1×). The degree of labeling (DOL) was calculated using the following equation:

$$DOL = \frac{{A}_{dye} {e}_{IgG}}{( {A}_{280} - {A}_{dye} {C}_{280} ) {e}_{IgG}}$$

where A_{dye} is the maximum monomeric absorbance of heptamethine, eIgG is the molar extinction coefficient of IgG at 280 nm, which equals to 200,000 M⁻¹ cm⁻¹, A280 is the absorbance of the conjugate at 280 nm, e_{dye} is the molar extinction coefficient of the dye, C280 is the correction factor of the dye at 280 nm, which is determined by A280/A_{Imax} of a free dye absorbance.

Fluorescence of mAb. Fluorescence of IgG-SAT-IR-746, IgG-SAT-IR-758 and IgG-IRDye800 were calculated using 96-Well Optical-Bottom Plates (Thermo Scientific™ Nunc). mAb-fluorophore conjugates (10 μL) were diluted with PBS pH 7.2 (90 μL) and fluorescence was measured at absorbance max of each conjugate. Fluorescence reading was measured three times.

Biological stability. SAT-IR-746 (10 μM) was tested for stability in pH (5.5 and 7.2) and serum (Mili-Q H₂O+10% FBS) for 18 h. The stability of the compound was monitored by observing the absorbance of the probe every 30 mins.

Procedure of different salt concentrations. All salt solutions were prepared at a concentration of 154 mmol/L for each salt and were created by calculating the required mass to dissolve in 50 mL of Milli-Q water.

$$g = \frac{[ M ] \times V \times M}{1000}$$

where g=grams of salt, [M]=mol/L of solution, V=solution volume (mL), M=molar mass of salt (g/mol).

Solutions were then used to read absorbance 20 μM dye concentrations in both symmetrical (SATIR-758) and unsymmetrical (SAT-IR-746). All absorbance reading were measured using JASCO spectrophotometer using a quartz cuvette with a 10 mm path length. A 500 mM solution of CaCl₂ was prepared, and then 6 two-fold serial dilutions were performed until a final concentration of 7.8125 mM was achieved. Absorbance was then read at 20 μM dye concentrations for both SAT-IR758 and SAT-IR-746 at 500 mM of CaCl₂ and for each two-fold dilution. All absorbance readings were measured using JASCO spectrophotometer using a quartz cuvette with a 10 mm path length.

Quantitative immunofluorescence microscopy. HeLa cells (ATCC CCL-2) were cultured in Dulbeco's Modified Eagle Medium (DMEM; Gibco 10313-021) supplemented with 10% (vol/vol) fetal bovine serum, 1% (vol/vol) GlutaMAX (Gibco 35050-061), 1% (vol/vol) Penicillin-Streptomycin (Gibco 15140-122), and maintained at 37° C., 5% CO₂. HeLa monolayers grown on glass coverslips were fixed in 4% paraformaldehyde at room temperature, before immunolabeling. Rabbit monoclonal anti-H3S10P (D2C8)(#3777, Cell Signaling Technology) was used to label mitotic chromatin for three hours at room temperature at 1:800 dilution in 2% BSA. After PBS washes, the monolayers were incubated with Hoechst 33342 and donkey anti-rabbit secondary antibodies conjugated to either AlexaFluor750, SAT-IR-746, or IRDye800, diluted to 3 μg/mL in a 2% BSA solution for one hour at room temperature.

Mitotic nuclei in prometaphase were identified based on Hoechst-labeled DNA morphology, and confocal images were acquired on a Leica SP8 with HC PL APO 63×/1.40 oil objective. Acquisition settings (638 nm laser at 2%, PMT gain at 1000, 4× digital zoom) were identical for every image acquired with all three fluorophores of interest. Images were batch processed and segmented in Cell Profiler (4.2.8)², and the mean fluorescence intensity of the H3S10P signal was determined for each prometaphase nucleus.

Hemolysis Assay. The hemolytic potential of SAT IR-746 dye was evaluated using porcine red blood cells (RBCs) purchased from Innovative Research, Inc. SAT-IR-746 was dissolved in PBS (1×) to obtain final concentrations of 100, 50, 10, 5, and 1 μg/mL. For each condition, 1 mL of dye solution was aliquoted into individual 1.5 mL centrifuge tubes. A volume of 50 μL of 10% packed porcine RBCs was then added to each tube and gently mixed.

Two control samples were prepared in parallel: a negative control containing 1 mL of PBS (1×) and a positive control containing 1 mL of DI water, each mixed with 50 μL of 10% RBC suspension. All tubes were incubated at 37° C. for 1 hour, followed by centrifugation at 3000 rpm for 20 minutes to pellet intact RBCs. The absorbance of the supernatant was measured at 540 nm to quantify hemoglobin release. The percentage hemolysis was calculated relative to the positive control using the following formula:

$$Hemolysis ( \% ) = \frac{{Ab}_{sample} - {Ab}_{background}}{{Ab}_{control} - {Ab}_{background}}$$

Tissue penetration profiling assessment. A stock solution for the tumor-mimicking phantoms was prepared by combining 720 mg of type-A gelatin, 150 mg of porcine hemoglobin, 900 μL of 20% intralipid, and 17.1 mL of PBS1×. Gelatin was first dissolved in PBS by heating at 60° C. with constant stirring at 950 rpm for 30 minutes. Once fully dissolved, hemoglobin and intralipid were added to the solution. The mixture was thoroughly inverted, vortexed, and stirred again at 60° C. for an additional 10 minutes at 950 rpm to ensure homogeneity. For each tumor-mimicking phantom, 1.5 mL of the stock solution was combined with 500 μL of SAT-IR-746 solution at different concentrations (1000-10 μg/mL). Control phantom was prepared by substituting the stock solution with 500 μL of PBS 1×. Each 2 mL mixture was then poured into silicone mold wells and frozen for at least one hour to achieve solidification.

To replicate the optical and structural properties of human tissues, two types of porcine tissue were selected as analogs: pork belly for skin and pork tenderloin for muscle. These tissues were sourced from local supermarkets and uniformly sliced to a thickness of 2 mm using a laboratory deli slicer. To simulate varying tumor depths, tissue slices were stacked accordingly, up to 4 mm for skin to reflect the upper limit of human skin thickness, and variable depths for muscle to model subcutaneous or deeper-seated tumors. Tumor-mimicking phantoms were prepared as previously described and positioned in a well plate for imaging. Fluorescence imaging was performed using the IVIS imaging system, with excitation at 740 nm and emission at 790 nm for the bare well plate containing phantoms and the phantoms stacked with tissues at different thicknesses to evaluate the depth penetration ability of the SAT-IR-746.

### B. Results

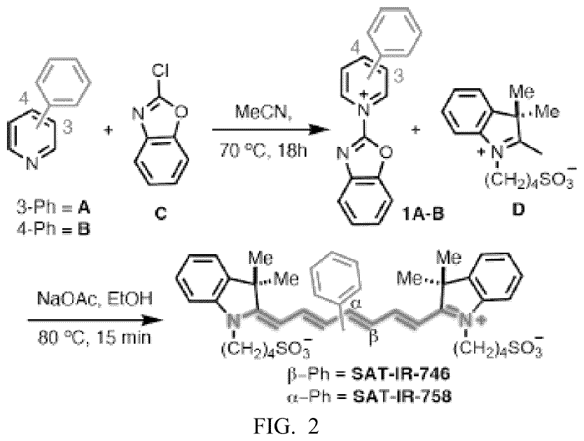

Development of polymethine substituted heptamethine cyanine. A common strategy for synthesizing unsymmetrical heptamethine cyanine involves having two different terminal indolenine moieties. However, this generally leads to low yields due to the uncontrolled reactivity of indolenine with the polymethine chain, resulting in the formation of symmetrical heptamethine cyanine instead (Zeng, Med. Chem. 2025, 68(8), 8174-89; Zhegalova, Contrast Media Mol. Imaging 2014, 9(5), 355-62; Babity, J. Org. Chem. 2025, 90(13), 4759-63; Lin, Bioconjug. Chem. 2002, 13(3), 605-10; Mujumdar, Bioconjug. Chem. 1993, 4(2), 105-11). To overcome this limitation, an alternative strategy was designed that introduces asymmetry directly on the polymethine chain through reactive pyridinium salts.^{43, 57, 58} To test our hypothesis that structural asymmetry disrupts π-π stacking interactions, we selected phenyl group as the easiest substitution possible on polymethine chain. These approaches enabled us to use a phenyl group at either the 3- or 4-position of the pyridine ring, resulting in unsymmetric and symmetric heptamethine cyanines, respectively (FIG. 2).

Both symmetrical and unsymmetrical heptamethine cyanines were synthesized through selective substitution on the polymethine chain to explore the effect of molecular symmetry on aggregation. In the first step, 3- or 4-phenyl substituted pyridines were reacted with 2-chlorobenzoxazole under nucleophilic substitution conditions to afford the corresponding pyridinium benzoxazole intermediates (1A and 1B, respectively), which can be easily precipitated in diethyl ether. In the second step, intermediates 1A and 1B were coupled with a sulfonated indolenine donor under basic conditions to yield the final SAT-IR (San Antonio-near-infrared) fluorophores. Reaction of the 3-phenyl substituted pyridinium (1A) afforded SAT-IR-746, the unsymmetrical dye, while coupling with the 4-phenyl variant (1B) produced the symmetrical analogue SAT-IR-758. Both fluorophores were obtained in moderate to good yields (70-80% yields) after reversed-phase chromatography.

TABLE 1
Photophysical properties of SAT-IR-746, SAT-IR-758 and IRDye
800 CW (Perkin Elmer) in organic and aqueous solvents. λ_{max, abs}(nm):
maximum absorbance, λ_{max, em} (nm): maximum emission, 1 = φ_{f}: fluorescence
Quantum yield, ε(M⁻¹ cm⁻¹): extinction coefficient 0.9% brine =
154 mM NaCl in MQ H₂O); 10% fetal bovine serum (H₂O + 10%
FBS).

| | | λ_{max, abs} | λ_{max, em} | ε | φ | Brightness |
|---|---|---|---|---|---|---|
| Compound | Solvent | (nm) | (nm) | (M⁻¹ cm⁻¹) | (%) | (φ × ε) |
| SAT-IR-746 | MeOH | 746 | 770 | 310,000 | 18 | 56,000 |
| | Mili-Q H₂O | 742 | 767 | 230,000 | 8.5 | 20,000 |
| | 0.9% Brine | 742 | 766 | 220,000 | 20 | 44,000 |
| | 10% FBS | 757 | 774 | 170,000 | 18 | 31,000 |
| SAT-IR-758 | MeOH | 758 | 782 | 200,000 | 14 | 28,000 |
| | Milli-Q | 752 | 776 | 200,000 | 14 | 36,000 |
| | 0.9% Brine | 754 | 777 | 140,000 | 16 | 22,000 |
| | 10% FBS | 773 | 790 | 130,000 | 13 | 17,000 |
| IRDye 800 CW | MeOH | 779 | 794 | 160,000 | 15 | 53,000 |
| | Mili-Q H₂O | 775 | 792 | 120,000 | 10 | 12,000 |
| | 0.9% Brine | 775 | 790 | 140,000 | 11 | 15,000 |
| | 10% FBS | 790 | 798 | 110,000 | 12 | 13,000 |

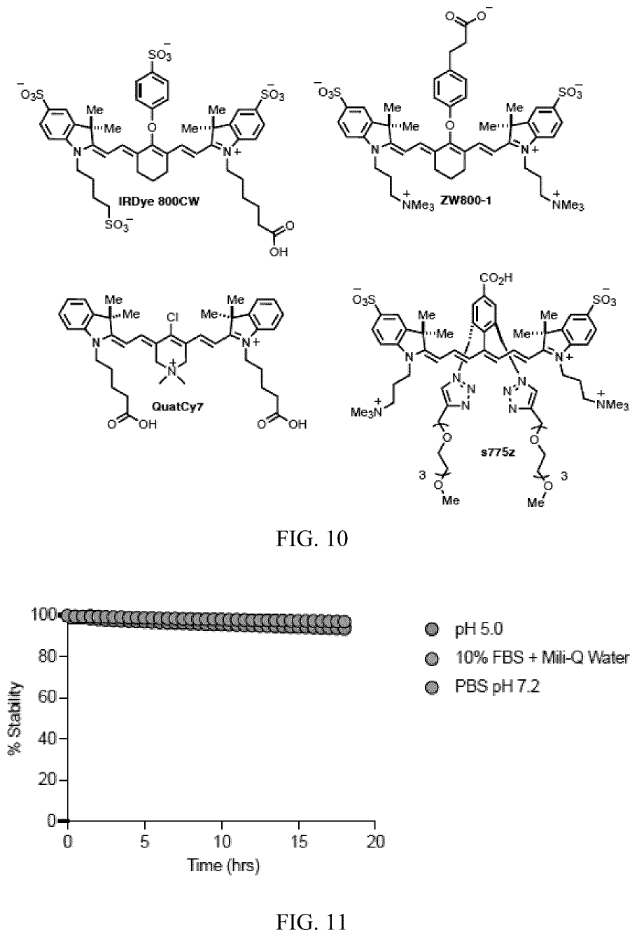

Photophysical properties of substituted heptamethine cyanine. The photophysical properties of SAT-IR-746 and SAT-IR-758 were evaluated in methanol (organic solvent) and biologically relevant solvents (MiliQ-H2O, 0.9% brine, and water supplemented with 10% fetal bovine serum) and compared with the clinically used dye IRDye 800CW (Table 1; FIG. 10). In methanol, SAT-IR-758 exhibited a maximum absorbance at 758 nm, while SAT-IR-746 showed a 12 nm blue shift with a maximum absorbance at 746 nm. However, brightness of SAT-IR-746 was higher than that of SAT-IR-758 in methanol. The extinction coefficients of SAT-IR fluorophores were higher in methanol compared to aqueous environments due to water's strong hydrogen-bonding network and higher polarity.⁵⁹ As expected, both dyes exhibited extinction coefficients on the order of 10⁵ M⁻¹ cm⁻¹ and quantum yields of 10-20% in aqueous solvents. SAT-IR-746 had higher brightness than SAT-IR-758 and IR-Dye800-CW in 0.9% brine, and 10% fetal bovine serum (FBS).

Testing for aggregation in different solvents. The aggregation behavior of two Cy7 derivatives, SAT-746 and, SAT-IR758, was assessed in a range of media that mimic different environments: methanol (a polar organic solvent that minimizes dye aggregation); Milli-Q H₂O (an aqueous, salt-free medium); 0.9% brine (154 mM NaCl, mimics physiological ionic strength); and 10% FBS in H₂O (a protein-rich environment representative of biological conditions where Cy7 dyes can interact with serum proteins). The absorbance was measured at increasing concentration (0.5-20 μM) in the media. In methanol, both fluorophores displayed a well-defined primary absorption peak near their λmax (˜750 nm) along with a secondary shoulder peak (˜700 nm). For symmetric SAT-IR-758, however, the shoulder peak became more pronounced in Milli-Q water as the concentration increased, is indicative of H-aggregation (parallel face-to-face stacking of Cy7). In 0.9% brine, the absorption spectrum of SAT-IR-758 broadened considerably, suggesting the formation of irregular aggregates due to the increased ionic strength. Under serum conditions, the shoulder peak disappeared, likely due to binding interactions between the dye and serum albumin, which inhibit aggregation. In contrast, SAT-746 maintained a consistent absorption profile across all organic and polar media. Its λmax and peak ratios remained stable regardless of the solvent, indicating that unsymmetric derivative resists aggregation even at higher concentrations and aqueous environments.

Salt effect on SAT-IR-746 and SAT-IR-758. Heptamethine cyanine dyes are known for their strong tendency to self-associate in aqueous environments, forming aggregates that dramatically alter their photophysical behavior.⁶⁰⁻⁶³ This aggregation in fluorophore arises from the extended polymethine chains and hydrophobic aromatic cores, which promote π-π stacking and hydrophobic interactions in water. The aggregates cause blue-shift in absorption bands and quenches fluorescence compared to monomeric species. Moreover, adding inorganic salts or increasing fluorophore concentration both enhances the self-association of fluorophores in aqueous conditions.

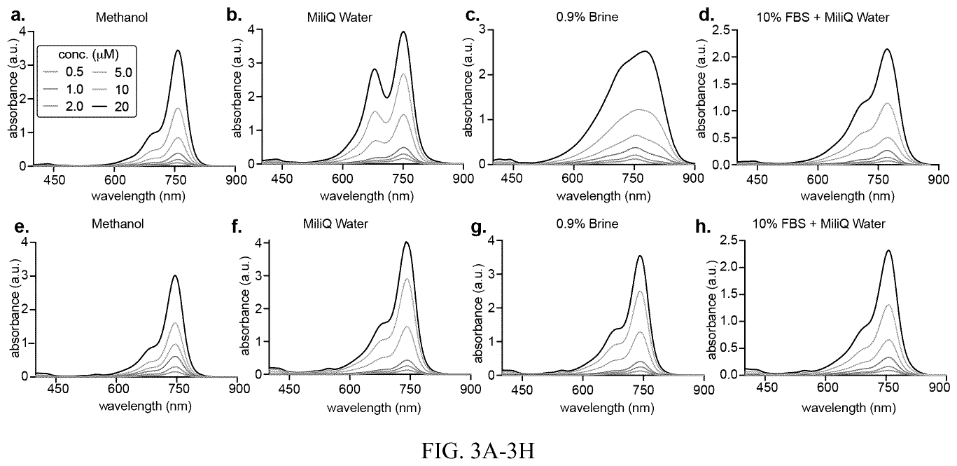

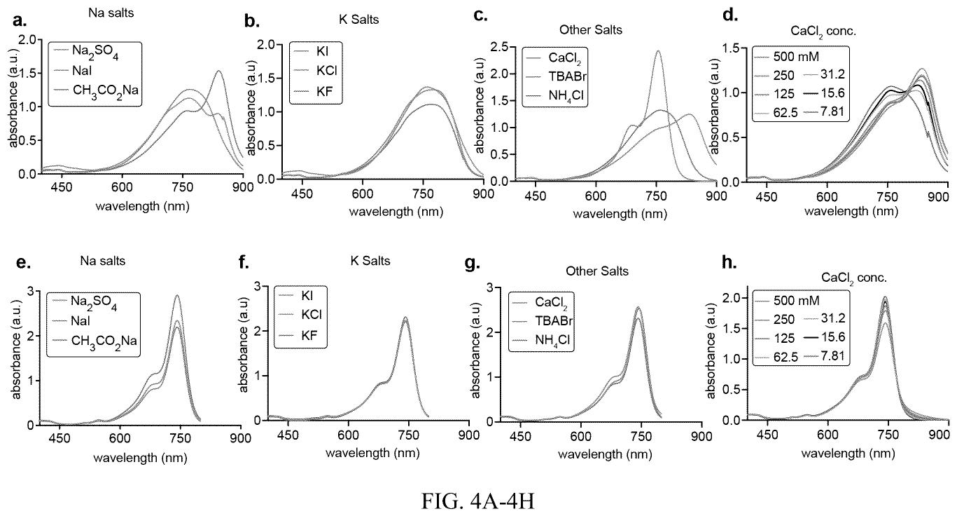

Increasing concentrations of salt raises the effective dielectric constant which affects aggregation in water. To evaluate this effect, we examined the absorbance behavior of SAT-IR-746 and SAT-IR-758 at three concentrations (10, 15, and 20 μM). The concentrations were selected based on observed aggregation in MQ water (FIG. 3). The fluorophores were tested in various salt solutions, including sodium salts (Na₂SO₄, NaI, CH₃CO₂Na), potassium salts (KI, KCl, KF), and other common salts (CaCl₂, TBABr, NH₄Cl). SAT-IR-758 displayed a broad absorbance profile at all tested concentrations in the various salts with no distinct primary peak or secondary shoulder (FIG. 4A-4D). This behavior may result from a random, high-order assembly of cyanine molecules in the solvent. Interestingly, a red shift in the absorbance of SAT-IR-758 was observed in sodium acetate and calcium chloride which indicate multivalent cations (Ca²⁺) induced more extensive aggregation than monovalent salts at equivalent ionic strengths. Additionally, there was a red shift in SAT-IR-758 (FIG. 4H) while increasing CaCl₂ from 7 mM to 500 mM.

The unsymmetrical dye SAT-IR-746 showed no significant changes in absorbance across all tested concentrations and salt solutions (FIG. 4E-4G). Additionally, increasing CaCl₂ from 7 mM to 500 mM did not affect the absorbance of SAT-IR-746 (FIG. 4H). This suggests that the structural asymmetry of the polymethine backbone effectively disrupts the π-π stacking interactions and reduces the propensity for dye-dye self association, even in surroundings with elevated ionic strength. The absence of spectral shifts or broadening supports the conclusion that SAT-IR-746 remains monomeric and optically stable in solution. These results highlight the potential of unsymmetrical cyanines as NIR probes for biological applications where varying ionic conditions are often encountered.

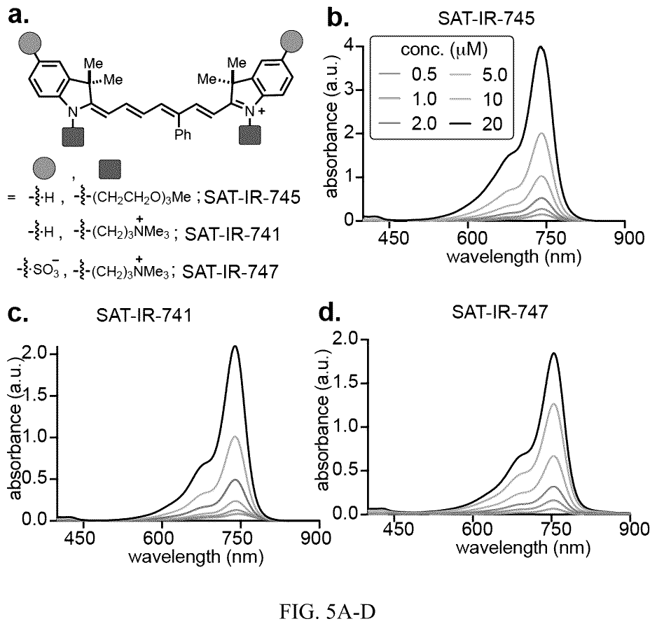

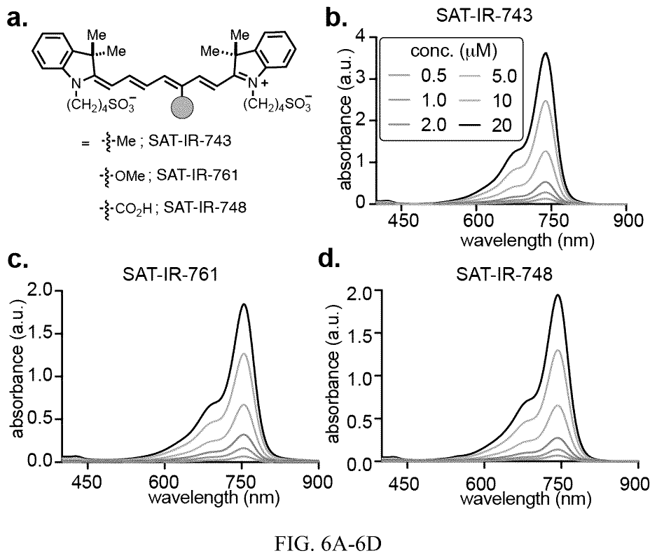

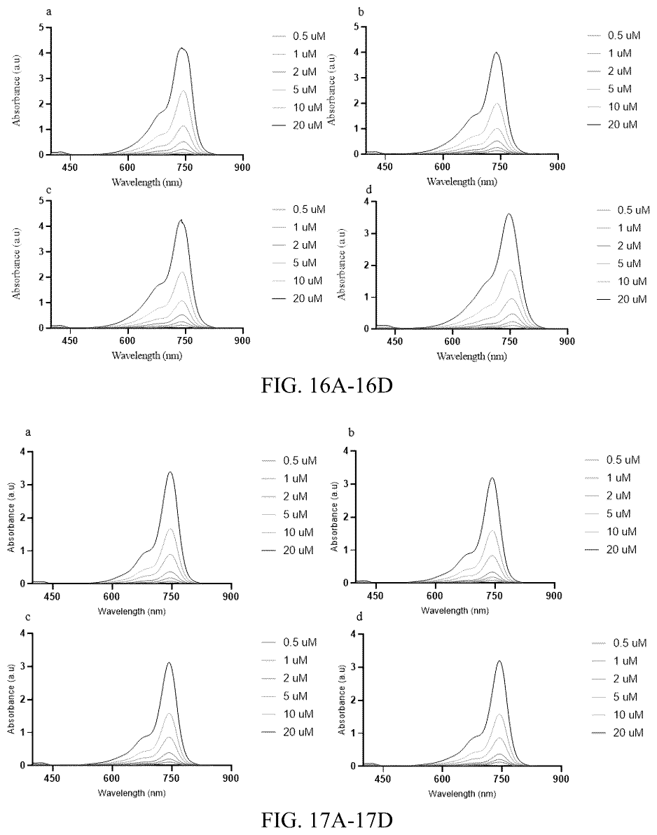

The methods have broad application to other substituted indoles and polymethine substituents. The asymmetric strategy can be applied broadly across heptamethine cyanine scaffolds. To test this, two libraries of unsymmetrical fluorophore were generated by varying both the indolenine (FIG. 5) substituents and the polymethine chain substitution (FIG. 6). Specifically, hepatmethine cyanines were synthesized with three different indolenine that are commonly used in literature, i.e., PEGylation, quaternary amine and zwitterionic indole. These indoles were reacted with compound 1A to give SAT-IR-745, SAT-IR-741, and a SAT-IR-747 respectively. All dyes were assessed for aggregation in Milli-Q water (FIG. 5) and 0.9% brine (FIG. 15-FIG. 17). Each fluorophore exhibited a strong absorption maximum near 750 nm, with only slight variations in peak position across the series. Importantly, none of the dyes showed evidence of aggregation in aqueous media. Moreover, the photophysical properties (Table 2) remained remarkably consistent when measured in both organic (methanol) and aqueous solvents (water, brine and FBS).

TABLE 2
Summary of the photophysical properties for quaternary amine (SAT-IR-741),
PEGylated (SAT-IR-745), and zwitterionic (SAT-IR-747), b-methyl (SAT-IR-
743), b-methoxy (SAT-IR-761), and b-carboxy (SAT-IR-748) derivatives.

| | | λ_{max, abs} | λ_{max, em} | ε | φ_{f} | |
|---|---|---|---|---|---|---|
| Compound | Solvent | (nm) | (nm) | (M⁻¹ cm⁻¹) | (%) | Brightness |
| SAT-IR-741 | MeOH | 741 | 762 | 130,000 | 20 | 26,000 |
| | Mili-Q H2O | 739 | 762 | 120,000 | 9.0 | 11,000 |
| | 0.9% Brine | 742 | 762 | 120,000 | 8.0 | 9,600 |
| | 10% FBS | 743 | 761 | 130,000 | 6.0 | 7,800 |
| SAT-IR-745 | MeOH | 745 | 768 | 260,000 | 14 | 36,000 |
| | Mili-Q H2O | 741 | 768 | 270,000 | 6.0 | 16,000 |
| | 0.9% brine | 741 | 770 | 270,000 | 8.0 | 22,000 |
| | 10% FBS | 754 | 778 | 240,000 | 9.0 | 22,000 |
| SAT-IR-747 | MeOH | 747 | 770 | 190,000 | 9.0 | 17,000 |
| | Mili-Q H2O | 744 | 766 | 170,000 | 10 | 17,000 |
| | 0.9% brine | 743 | 765 | 180,000 | 6.0 | 11,000 |
| | 10% FBS | 743 | 765 | 180,000 | 5.0 | 9,000 |
| SAT-IR-743 | MeOH | 743 | 765 | 340,000 | 9.0 | 31,000 |
| | Mili-Q H2O | 739 | 762 | 270,000 | 4.0 | 11,000 |
| | 0.9% Brine | 738 | 760 | 240,000 | 4.0 | 10,000 |
| | 10% FBS | 758 | 768 | 340,000 | 7.0 | 24,000 |
| SAT-IR-761 | MeOH | 761 | 785 | 150,000 | 17 | 26,000 |
| | Mili-Q H2O | 754 | 777 | 130,000 | 19 | 25,000 |
| | 0.99% brine | 755 | 776 | 120,000 | 20 | 24,000 |
| | 10% FBS | 767 | 782 | 120,000 | 10 | 12,000 |
| SAT-IR-748 | MeOH | 748 | 769 | 160,000 | 19 | 30,000 |
| | Mili-Q H2O | 743 | 764 | 130,000 | 7.0 | 9,000 |
| | 0.9% brine | 743 | 766 | 130,000 | 7.0 | 8,000 |
| | 10% FBS | 751 | 765 | 120,000 | 8.0 | 10,000 |

λ_{max, abs} (nm): maximal absorption,
λ_{max, em} (nm): Maximal emission.
e (M⁻¹ cm⁻¹): Extinction coefficient.
1 = φ_{f}: relative fluorescence quantum yield (ICG as a reference, φ_{f} for ICG = 0.13 in DMSO.
ε(M⁻¹ cm⁻¹): extinction coefficient 0.9% brine = 154 mM NaCl in MQ H₂O);
10% fetal bovine serum (H₂O + 10% FBS)

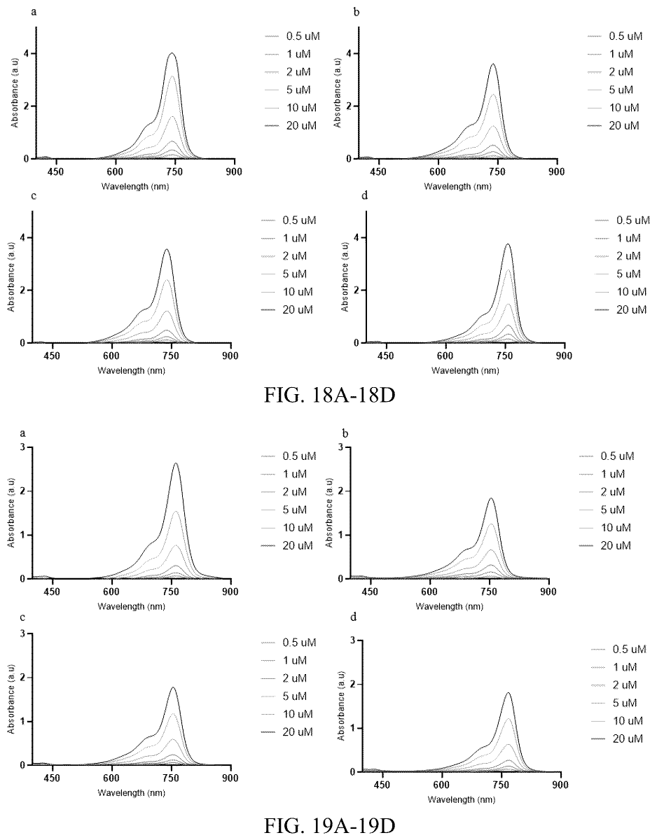

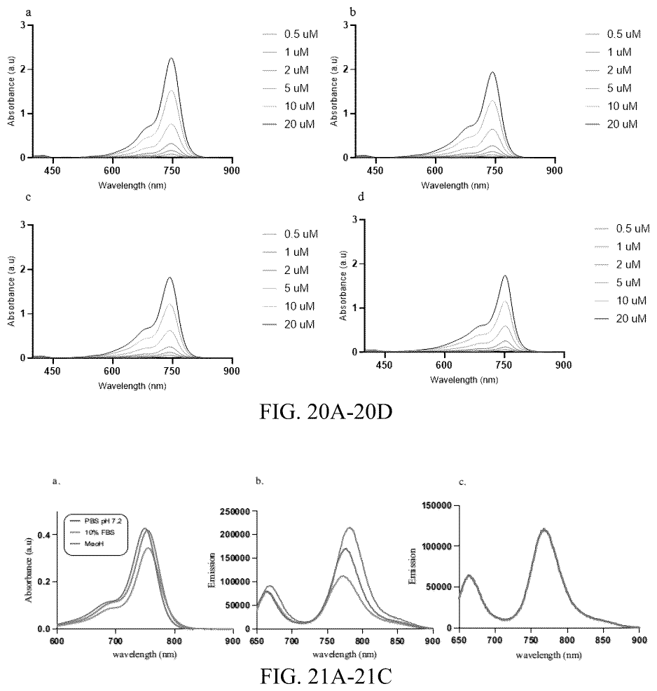

The impact of diverse substituents on the polymethine backbone were assessed. Using the pyridinium benzoxazole approach, methyl (aliphatic group), methoxy (electron-donating group) and carboxylic acid (electron-withdrawing group) were introduced at the b-position of the polymethine chain. Three-substituted pyridines were converted to activated pyridinium benzoxazole intermediates via reaction with 2-chlorobenzoxazole. These intermediates were condensed with sulfonated indolenine (compound D) to give SAT-IR fluorophores. The b-substituted polymethine scaffold was evaluated for aggregation in Milli-Q water (FIG. 6) and methanol, 0.9% brine, and 10% FBS (FIG. 18-FIG. 20). All derivatives displayed similar absorbance at wavelengths between 740 and 760 nm and showed no evidence of aggregation. These findings support the premise that having asymmetry in heptamethine cyanine represent a general strategy scaffold to construct non-aggregation fluorophores that can be broadly applied to substituted indolenine or polymethine modifications.

Biological stability. Time-course incubations were performed under physiologically relevant conditions to evaluate the biological and pH stability. SAT-IR-746 was incubated with biological media (containing 10% FBS), acidic and neutral pH (5.0 and 7.2). Absorbance was measured after every 30 mins for 18 h (FIG. 11). SAT-IR-746 remained stable across the tested conditions, exhibiting negligible degradation over 18 h. These results confirm that structural asymmetry prevents aggregation without impacting the stability in biological relevant environments, suggesting that SAT-IR-746 is well suited for long-term imaging studies in biological environments.

Hemocompatibility Assessments of SAT-IR-746. For any NIR fluorophore to be considered suitable for in vivo imaging it must exhibit excellent biocompatibility—including hemocompatibility. Heptamethine cyanines can promote aggregation and lead to undesirable interactions with blood components. These interactions may trigger hemolysis, affect clotting mechanisms, or activate the immune response, ultimately compromising safety and imaging reliability.

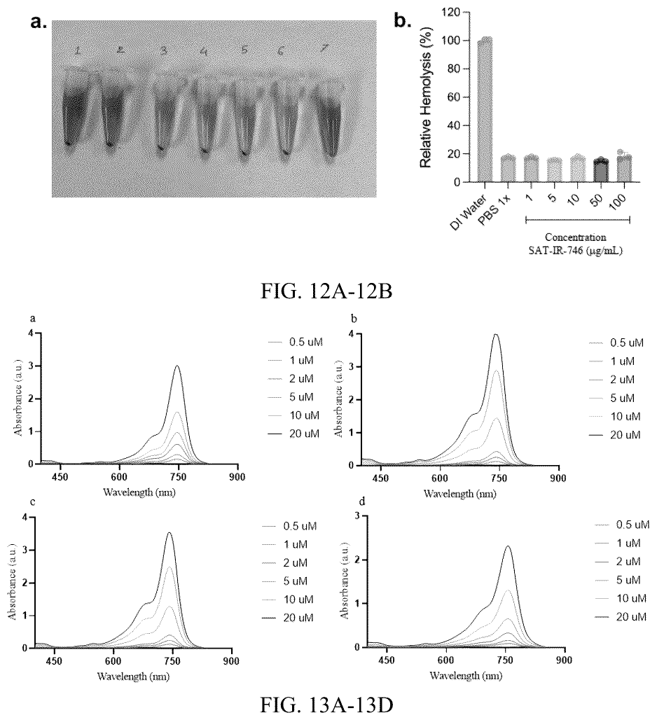

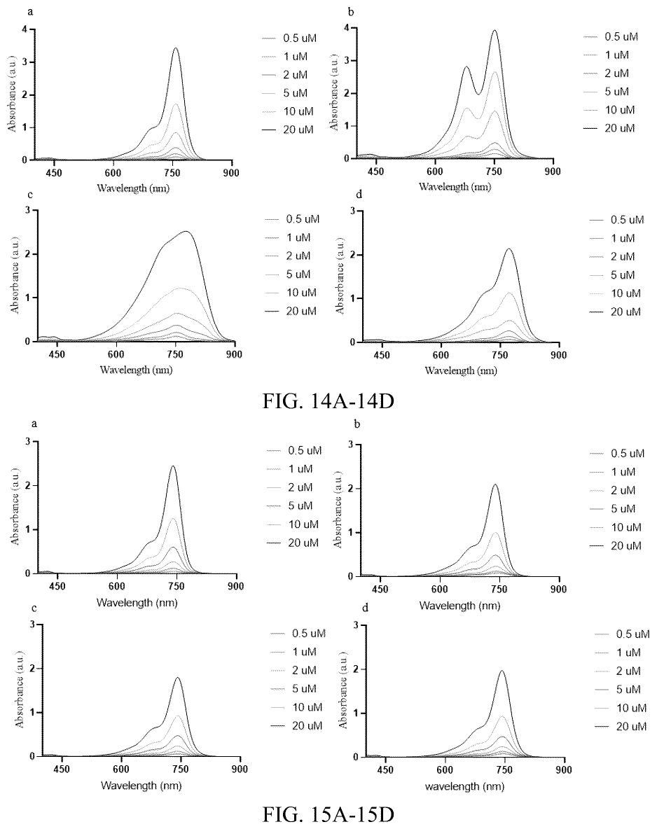

To evaluate the hemocompatibility of SAT-IR-746, hemolysis levels were quantified following the incubation of porcine red blood cells (RBCs) with increasing concentrations of the dye (1, 5, 10, 50, and 100 μg/mL). Phosphate-buffered saline (1×PBS) and deionized (DI) water were used as negative and positive controls, respectively. In all SAT-IR-746-treated samples, intact RBC pellets were clearly visible at the bottom of the centrifuge tubes, indicating minimal membrane disruption (FIG. 12A). In contrast, the DI water-treated group exhibited complete hemolysis, as evidenced by the absence of a pellet and a uniformly red supernatant, while the PBS control showed a compact pellet and clear supernatant, confirming negligible hemolysis.

Quantitative analysis further confirmed that SAT-IR-746 induced hemolysis at all tested concentrations at levels significantly lower than the positive control (FIG. 12B). These results demonstrate that SAT-IR-746 does not cause appreciable red blood cell lysis under physiologically relevant conditions and is hemocompatible. Importantly, the concentrations evaluated were consistent with those commonly used in in vivo mouse imaging studies, supporting the dye's potential suitability for further preclinical applications.

Bioconjugation strategy. Biomolecules such as proteins are generally conjugated to fluorophores for real-time visualization and tracking of their localization, movement and interactions in biological systems. The fluorescence from bioconjugates assists in quantification of molecular interactions, determining cellular localization, and dynamics in biological environments. The amino acid lysine is used for labeling due to its nucleophilic F-amino group (pKa˜10.5).⁶⁴ Further, lysine is abundantly present on the protein surface in aqueous media due to its positive charge at physiological pH enabling modification approaches. IgG antibodies contain ˜90 lysines (PDB: 1HZH65; 1IGY66). This high density of lysines poses a challenge in conjugating near-infrared fluorescent dyes such as hepatmethine cyanine, as the extended polymethine chain and planar aromatic terminals favor π-π stacking when positioned in closed proximity to one another. Several literature examples report that labeling the IgG molecule with more than one heptamethine cyanine dye can lead to dye-dye interactions and self-assembly on the protein surface that causes fluorescence quenching and impacting the biodistribution of the antibody in vivo.⁶⁷⁻⁷²

After confirming that unsymmetrical heptamethine cyanine dye SAT-IR-746 does not aggregate in aqueous solution, the conjugation to monoclonal antibodies (mAbs) without promoting aggregation on the protein surface was evaluated. The most common approach for lysine modification on proteins involves N-hydroxysuccinimide (NHS) esters.⁷³ To enable this conjugation strategy, a carboxylic acid functional group was introduced onto the SAT-IR dye scaffold, which could subsequently be converted into an NHS ester.

Specifically, 4-pyridin-[3-(E) or 4-(F)yl]-benzoic acid derivatives were coupled to 2-chlorobenzoxazole to get activated pyridinium benzoxazole intermediates (3E-F). The 3E-F intermediates were then condensed with sulfonated indolenine (compound D) to yield unsymmetrical Cy7 chromophores bearing a free carboxyl group—SAT-IR-746-CO₂H and SAT-IR-758-CO₂H, respectively. The resulting carboxylic acids were converted into NHS esters by reacting them with TSTU and NHS in anhydrous DMF in the presence of DIPEA, yielding SATIR-746-NHS and SAT-IR-758-NHS. These NHS-activated fluorophore (used at a 20-40 molar excess relative to protein) were incubated with goat IgG (0.2 mg) in 50 mM PBS (pH 7.4) for 2 hours at room temperature. After conjugation, excess unreacted fluorophore was removed using centrifugal filtration followed by dialysis. The degree of labeling (DOL) was determined by measuring absorbance at 280 nm (for protein) and the dye's maximum absorbance wavelength. IRDye800-NHS was used as a reference for comparison during antibody labeling experiments.

SAT-IR-746, SAT-IR-758, and the commercially available IRDye800CW were labeled at a similar degree of labeling for comparison study (FIG. 6B-6D). These IgG-fluorophore conjugates display a primary absorption peak centered around 750-800 nm that is characteristic of heptamethine cyanines. Additionally, a prominent shoulder peak was observed, which is commonly associated with H-aggregation^{37, 74, 75} in both IgG-IRDye800CW and IgG-SAT-IR-758 conjugates even at low DOL of 1. The shoulder peak became more pronounced with increasing DOL to ˜5, which indicates the onset of H-aggregation at higher labeling densities. In contrast, the SAT-IR-746 conjugates retained its monomeric spectral profiles even at high DOLs.

The aggregation tendency of the different IgG-fluorophore conjugates are quantitatively compared by plotting the ratio of absorbance at the main peak (monomer) to that at the shoulder peak (aggregates) versus DOL. As a reference point, DOL=0 is defined as the free fluorophore at a non-aggregating concentration (0.5 μM in 0.9% brine). It was observed that this monomer-to-aggregate absorbance ratio progressively decreased for both IgG-SAT-IR-758 and IgG-IRDye800CW as DOL increased. Notably, the steepest decline (approximately 50%) occurred between DOL 0 and DOL 1, which suggest aggregation becomes significant even at low labeling densities for these dyes. In contrast, the monomer-to-aggregate absorbance ratio of IgG-SAT-IR-746 did not show any decrease across the different DOLs. Lastly, fluorescence of the three IgG-fluorophore conjugates was compared at different DOLs (FIG. 6F). The fluorescence of IgG-SAT-IR-746 increased at higher DOL. However, IgG-IRDye800CW and IgG-SAT-IR-758 exhibited little to no enhancement, which is consistent with self-quenching from aggregation. These results validate the hypothesis that the unsymmetrical SAT-IR-746 scaffold is ideally suited for bioconjugation scenarios requiring high labeling densities without compromising fluorescence.

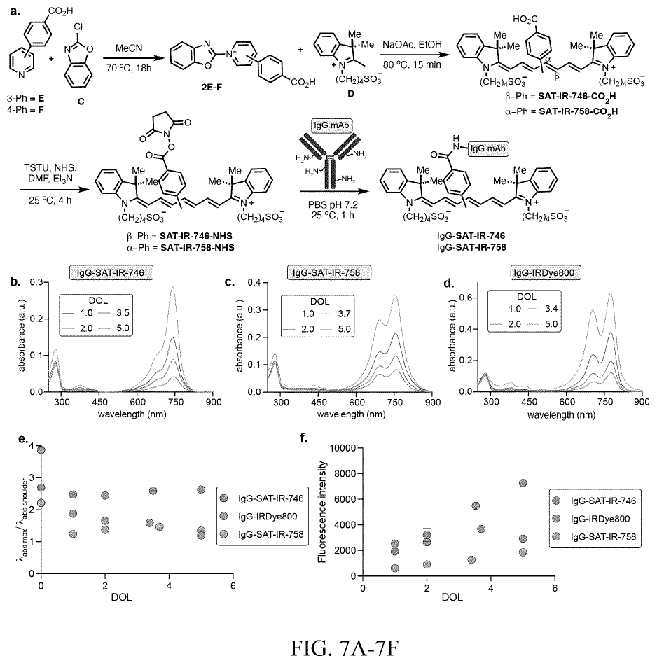

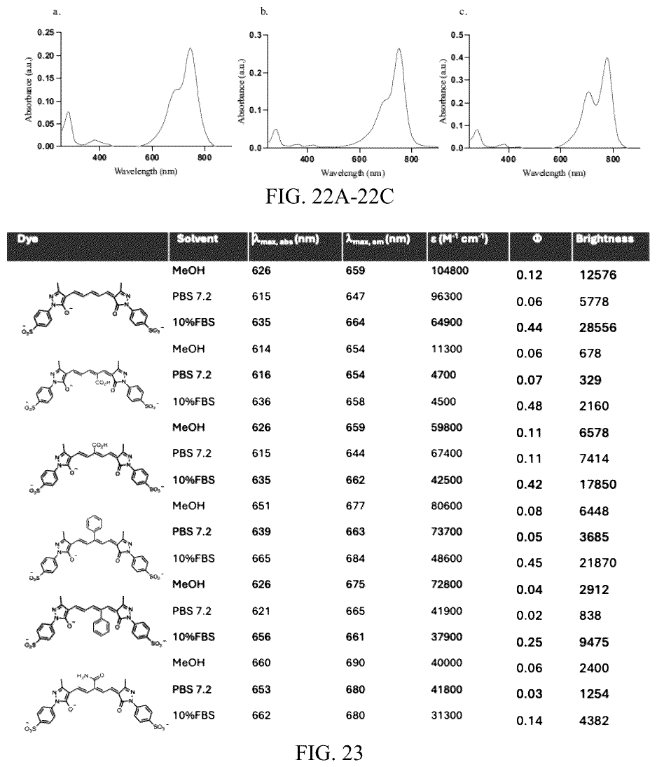

Immunofluorescence microscopy. The utility and performance of SAT-IR-746 was evaluated for use in quantitative immunofluorescence microscopy on a standard turnkey confocal microscope. Most confocal microscopes lack dedicated NIR lasers and instead rely on lasers near 630-638 nm. To perform a fair comparison of NIR fluorophores, monoclonal antibody specific for rabbit IgG (FIG. 22) was conjugated to SAT-IR-746, Alexa Fluor 750, or IRDye800, each with a similar degree of labeling (DOL ˜5), for use as secondary antibody. Significant aggregation was observed with the IRDye800-labeled antibody, while Alexa Fluor 750 displayed a single absorbance peak but showed two fluorescence emission peaks: one around 650 nm and another near 750 nm (FIG. 21). Comparative performance of the three NIR-coupled secondary antibodies was assessed using a rabbit primary antibody specific for histone H3 phosphorylated at serine 10 (H3S10P), a marker of mitotic chromatin that peaks during metaphase.⁷⁶ All three conjugates were used to image (FIG. 7A) and quantify (FIG. 7B) the fluorescence intensity of H3S10P-labeled mitotic chromatin. Using a 638 nm laser at 2% power for excitation, SAT-IR-746 produced the brightest fluorescence signal—slightly brighter than Alexa Fluor 750 and significantly brighter than IRDye800. These results demonstrate that SAT-IR-746 is a robust, non-aggregating fluorophore well-suited for multicolor immunofluorescence microscopy and quantitative bioimaging applications.

Ex Vivo Optical Tissue Permeation Profiling Assessment. SAT-IR-746 was evaluated in an ex vivo study to assess its fluorescence signal permeation through biologically relevant tissue layers, such as skin and muscle, to simulate conditions encountered during intraoperative imaging. Although most preclinical evaluations are conducted in small animal models such as mice, these models do not adequately mimic the greater tissue thicknesses present in human anatomy.⁷⁷ Murine tissue layers typically span only a few millimeters, whereas human tissue can reach several centimeters in depth, substantially altering photon penetration and fluorescence signal intensity.⁷⁸

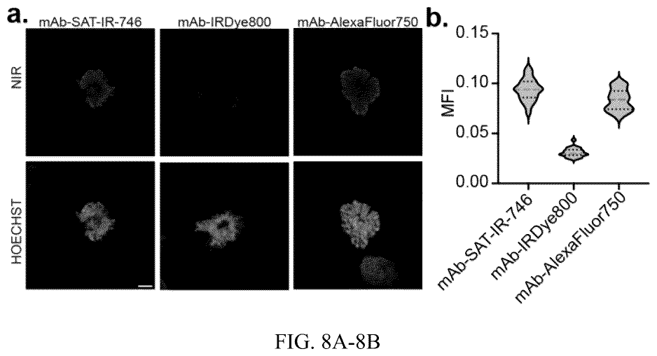

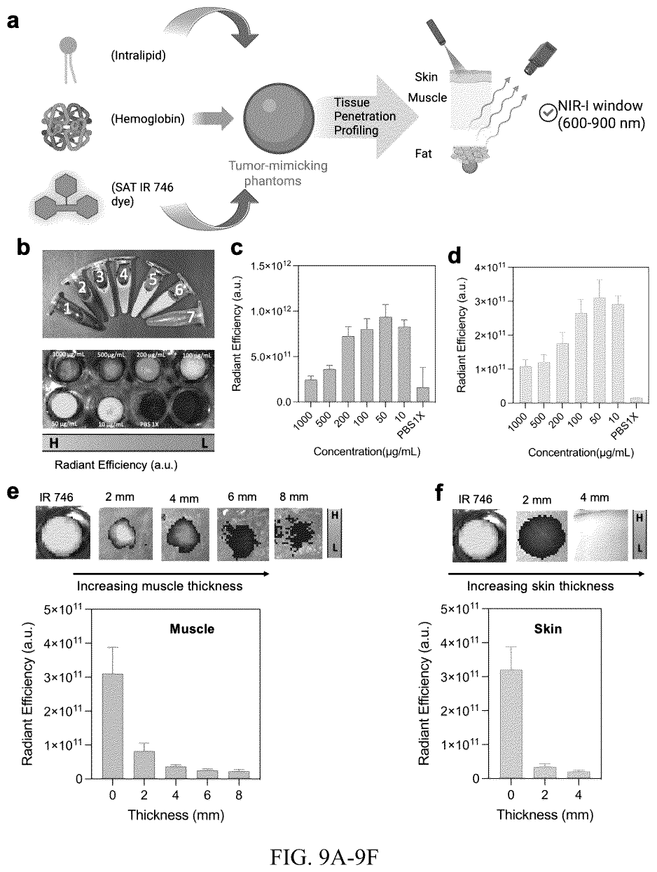

To address these translational limitations, three-dimensional tumor-mimicking phantom models designed to replicate the optical and structural properties of human tissues were employed.⁷⁹⁻⁸¹ These phantoms incorporate intralipid as the scattering medium, hemoglobin as the absorbing component, and SAT-IR-746 at biologically relevant concentrations to simulate the fluorescence of tumor tissue. Compared to theoretical simulations or intralipid-only phantoms, these engineered models offer greater experimental reproducibility, stability over time, and more physiologically accurate fluorescence distributions. To evaluate the fluorescence performance of SAT-IR-746 dye as a function of concentration, IVIS imaging was performed across a range of fluorophore concentrations (FIG. 8B). A noticeable reduction in signal intensity was observed at higher concentrations, particularly at 1000 μg/mL. The highest fluorescence output was observed at 50 μg/mL, beyond which signal attenuation occurred (FIG. 8C-8D).

Fluorescence imaging of SAT-IR-746 phantoms was performed using the IVIS system following tissue coverage with variable-thickness porcine skin and muscle (FIG. 8E). As expected, increasing tissue thickness led to a progressive decline in NIR signal intensity. The most notable reduction in signal was observed when the skin layers reached a thickness of 4 mm (FIG. 8F). Similarly, when SAT-IR-746 phantoms were overlaid with porcine muscle, a gradual reduction in fluorescence was observed, with sharper attenuation at 8 mm thicknesses (FIG. 8E). The emission peak of SAT-IR-746 at 790 nm, achieved at a 50 μg/mL concentration reduces spectral interference and enhances signal clarity and depth discrimination. These results signified the potential of SAT-IR-746 in fluorescence-guided surgical procedures where deeper tissue visualization is critical.

TABLE 3
Summary of DOLs for IgG-SAT-IR746 IgG-SAT-IR758-NHS and IgG-IRDye 800-
NHS conjugated with monoclonal IgG antibody. C₂₈₀ (protein concentration),
A_{dye} (Maximum absorbance of fluorophore), A₂₈₀ (absorbance of antibody),
ε_{dye} (Extinction coefficient of fluorophore in PBS pH 7.2) and DOL (Degree
of labeling). The equivalents used for antibody labeling of fluorophore
for IgG labeling: SAT-IR-746-NHS (DOL:Eq, 1:20, 2:30, 3.5:45 and 5:50)
for SAT-IR-758-NHS (1:18, 2:30, 3.5:40 and 5:80) and for IRDye800-
NHS (1:2, 2:4, 3.5:8 and 5:24)

| | IgG-SAT-IR746 | | | | IgG-SAT-IR758 | |
|---|---|---|---|---|---|---|
| C₂₈₀ | 0.49 | 0.55 | 0.50 | 0.73 | 0.74 | 0.80 |
| A_{Dye} | 0.04 | 0.09 | 0.15 | 0.29 | 0.08 | 0.13 |
| A₂₈₀ | 0.07 | 0.08 | 0.08 | 0.12 | 0.10 | 0.11 |
| ε_{Dye} | 130000 | 130000 | 130000 | 130000 | 140000 | 1400000 |
| DOL | 1.0 | 2.0 | 3.5 | 5.0 | 1.0 | 2.0 |

| | IgG-SAT-IR758 | | IgG-IRDye800 | | | |
|---|---|---|---|---|---|---|
| C₂₈₀ | 0.68 | 0.90 | 0.73 | 0.78 | 0.71 | 0.81 |
| A_{Dye} | 0.21 | 0.36 | 0.12 | 0.20 | 0.38 | 0.63 |
| A₂₈₀ | 0.10 | 0.14 | 0.10 | 0.11 | 0.11 | 0.10 |
| ε_{Dye} | 140000 | 140000 | 240000 | 240000 | 240000 | 240000 |
| DOL | 3.5 | 5.0 | 1.0 | 2.0 | 3.5 | 5.0 |

TABLE 4
Summary of DOLs of SAT-IR746-NHS (145 eq), AlexaFluor
750 (20 eq) and IRDYE800 (20 eq) conjugated
with Donkey-anti-rabbit antibody.

| | C₂₈₀ | A_{dye} | A₂₈₀ | ε_{dye} | DOLs |
|---|---|---|---|---|---|
| SAT-IR-746 | 0.47 | 0.217 | 0.076 | 130000 | 5.61 |
| Donkey-anti-rabbit | | | | | |
| AlexaFluor 750 | 0.29 | 0.255 | 0.051 | 290000 | 4.82 |
| Donkey-anti-rabbit | | | | | |
| IRDYE 800 | 0.46 | 0.398 | 0.081 | 240000 | 5.43 |
| Donkey-anti-rabbit | | | | | |

C₂₈₀ (protein concentration), A_{dye} (Maximum absorbance of dye), A₂₈₀ (absorbance of antibody), ε_{dye} (Extinction coefficient of dye in PBS pH 7.2) and DOLs(Degree of labeling).

### Example 3—Development of Conjugatable SAT-IR7 SAT-IR742-α-Cf₃

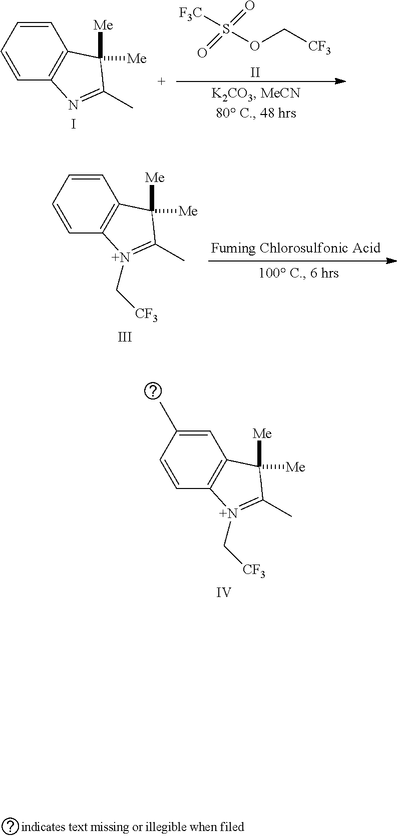

Compounds I and II were added to a 20 mL microwave vial along with Potassium bicarbonate (K₂CO₃) heated solution for 48 hrs at 80° C. The reaction products were dissolved in DCM, used separatory funnel and some water for a 90% Yield. Compound III and fuming chlorosulfonic acid heated at 100° C. for 6 hours were added. The reaction product was dissolved in MiliQ Water and purified using Reverse ISCO at 0-95% gradient to a 98% Yield.

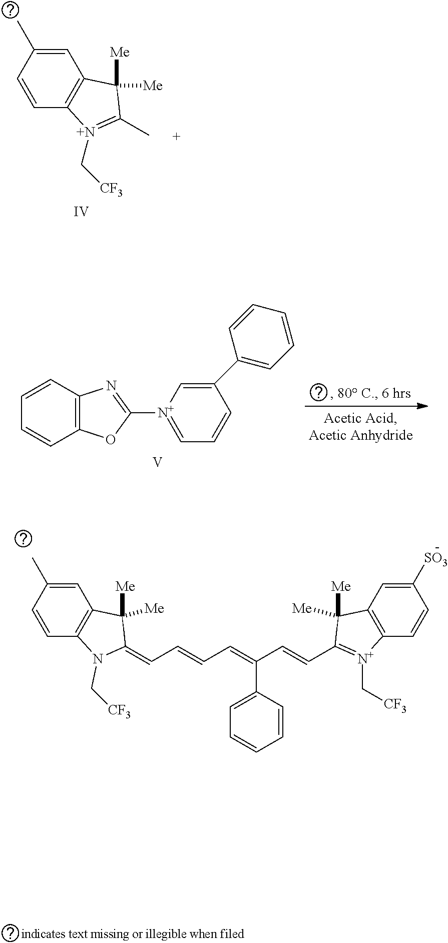

Compounds IV and V were added to a 20 mL vial as well as Sodium Acetate (NaOAc) as well as a 1:1 ratio of Acetic Acid and acetic anhydride. The solution was heated for 6 hours at 80° C. and precipitated in ether.

### Example 4—Development of Conjugatable SAT-IR7 SAT-IR742-α-Cf₃NHS Ester

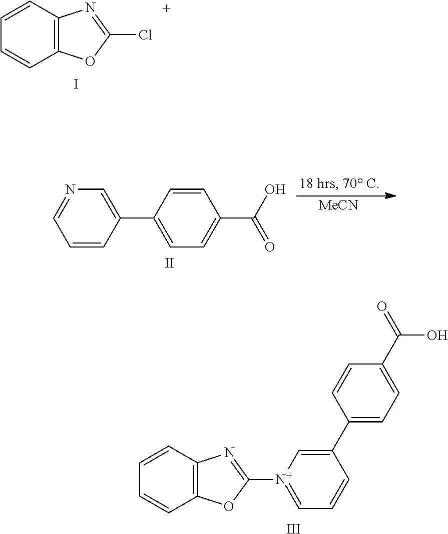

Compounds I and II were added to a 20 mL vial as well as Acetonitrile (MeCN) and heated solution at 70° C. for 18 hours. Followed by precipitated in ether, vortex, and centrifuged. Dried overnight. 68% yield

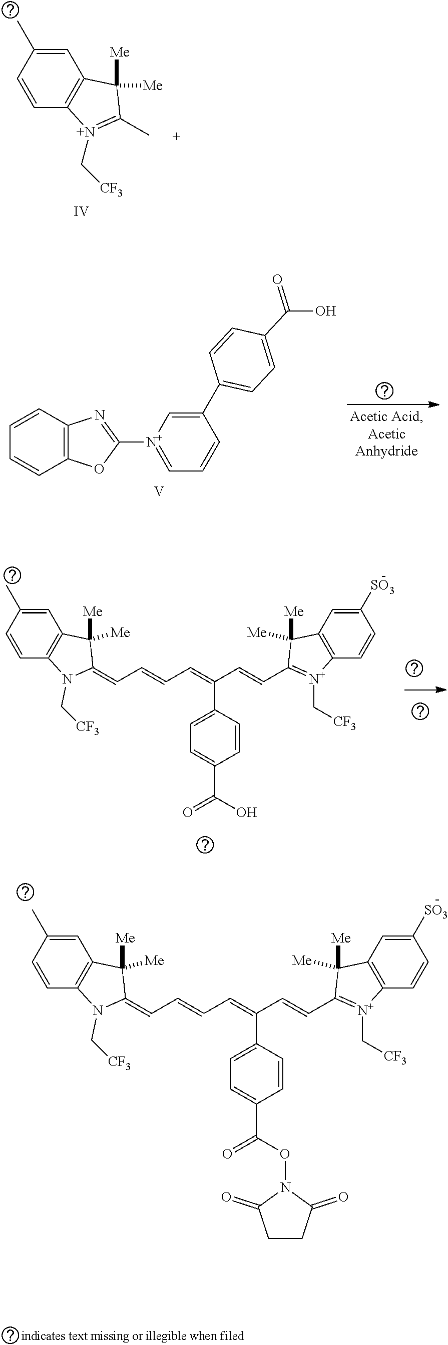

Compounds IV and V were added to a 20 mL microwave vial as well as added Sodium Acetate (NaOAc) and a 1:1 ratio of Acetic Acid and Acetic Anhydride heated solution for 6 hours at 80° C. Followed by precipitation in ether, vortexed, centrifuged, and dried overnight with a vacuum. Compound VI and TSTU, DMF, DIPEA were added to a 20 mL microwave vial and stirred at 25° C. for 30 minutes then added NHS Ester and continued stirring for 2 more hours. Followed by precipitation in ether and dried overnight with a vacuum.

### Example 5—Activation of Fluorogenic Probe by H₂O₂

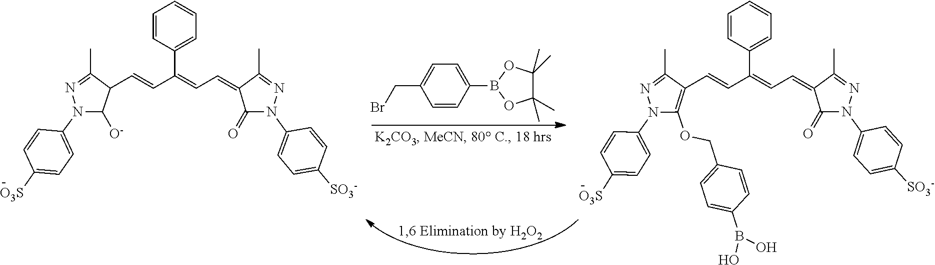

### Mechanism of Action

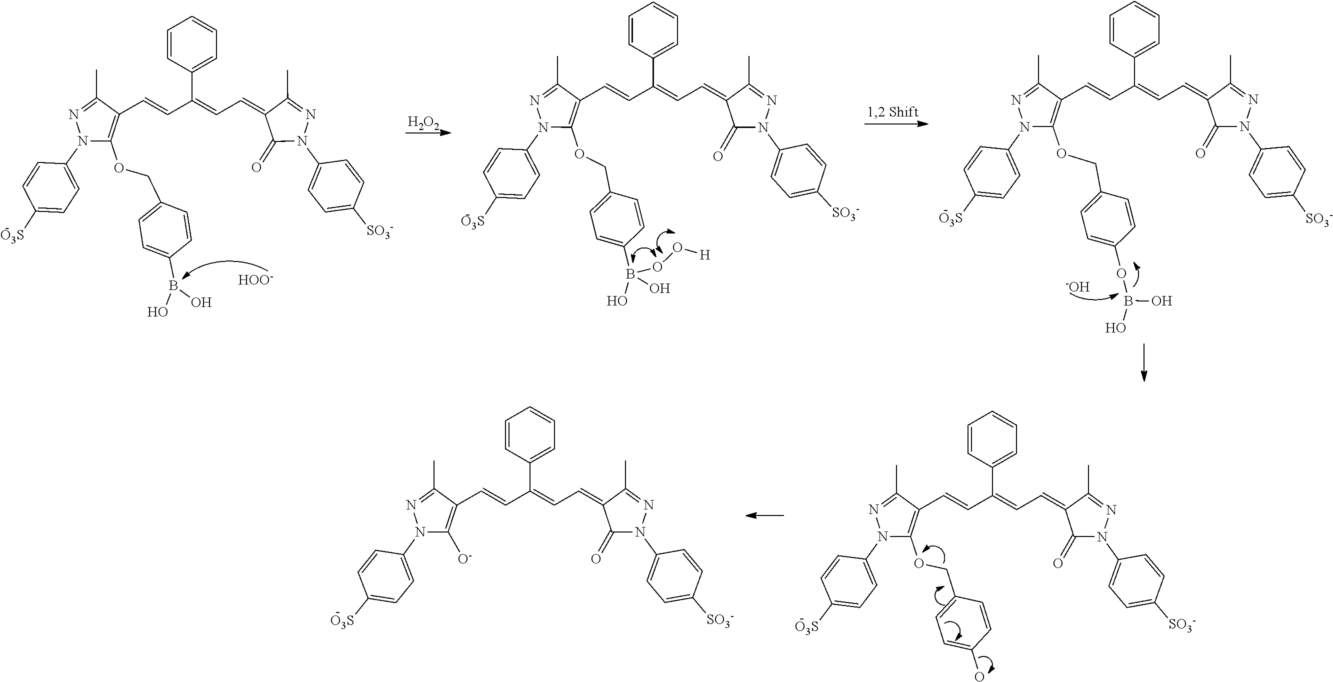

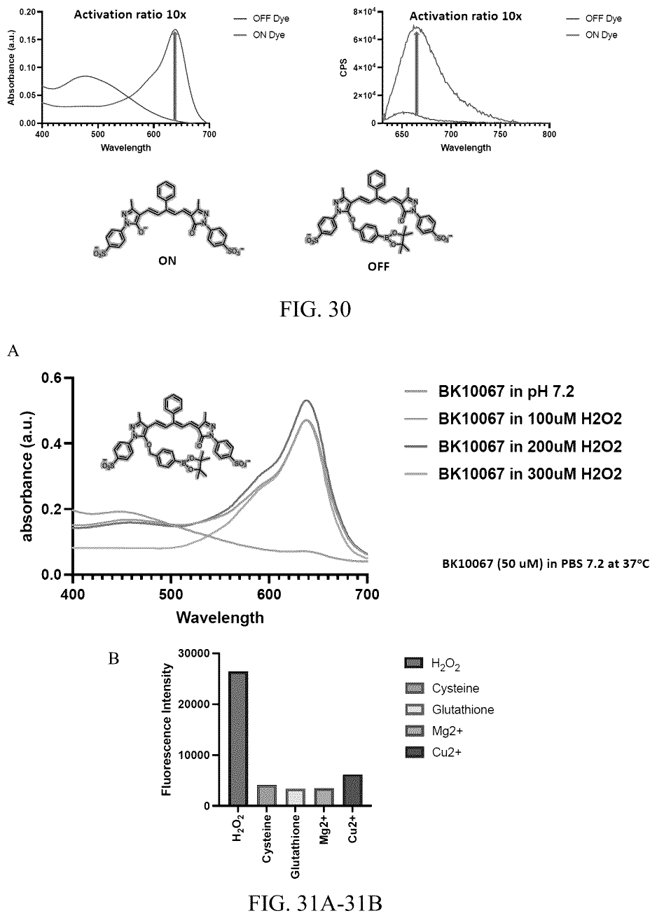

Results of BK10067 50 uM H₂O₂ Activation are shown in FIG. 31A. This probe was highly selective for H₂O₂ compared to other intracellular analytes as is shown in FIG. 31B.

### Example 6—Synthesis of Off-Dye

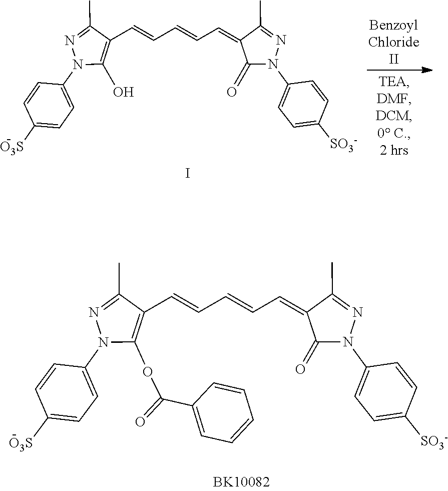

Method. 1. Add I with TEA in DMF & II in DCM into vial for 2 hrs@0° C. 2. Product formed—confirmed by LCMS 3. Removed vial, dried with N2 4. Purify with Dry loading ISCO reverse phase 0-50% B water/acetonitrile. 5. Perform pHPLC for further purification 6. Collect sample, perform stability test

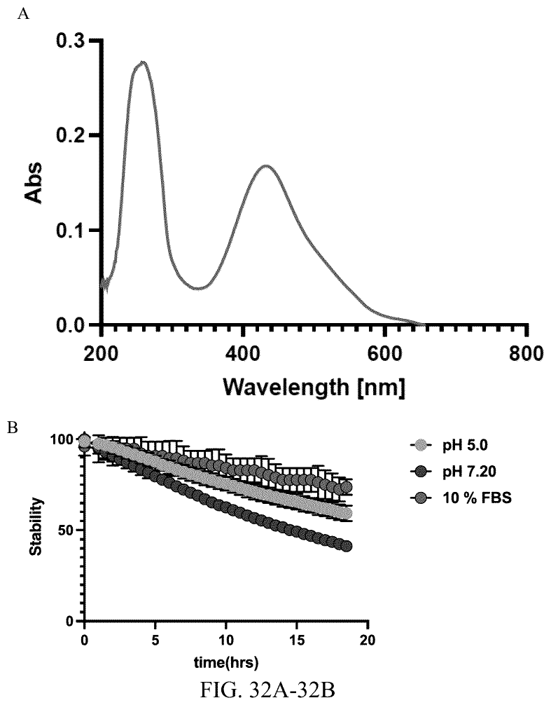

Results: UV-Vis data shoes that there is no absorbance at 600 nm region for full dye, only off dye absorbance at ˜450 nm is detected (FIG. 32A).

### Example 7—Synthesis of Fluorogenic Probe for Nitroreductase

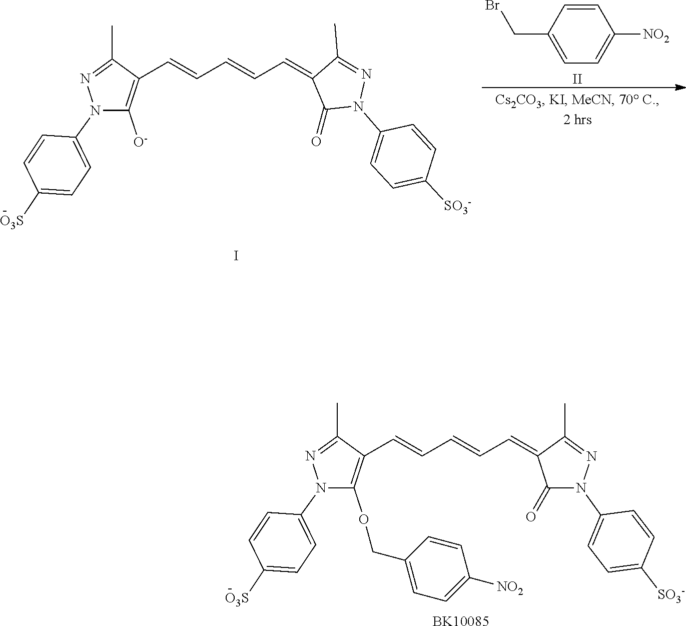

Method: 1. Add I & II in MeCN with Cs2CO3, KI into vial for 2 hrs@ 70° C. 2. Product formed—confirmed by LCMS 3. Remove vial, remove solvent by N2 drying 4. Dry loading ISCO Reverse phase 0-50% water/MeCN 5. Collect sample.

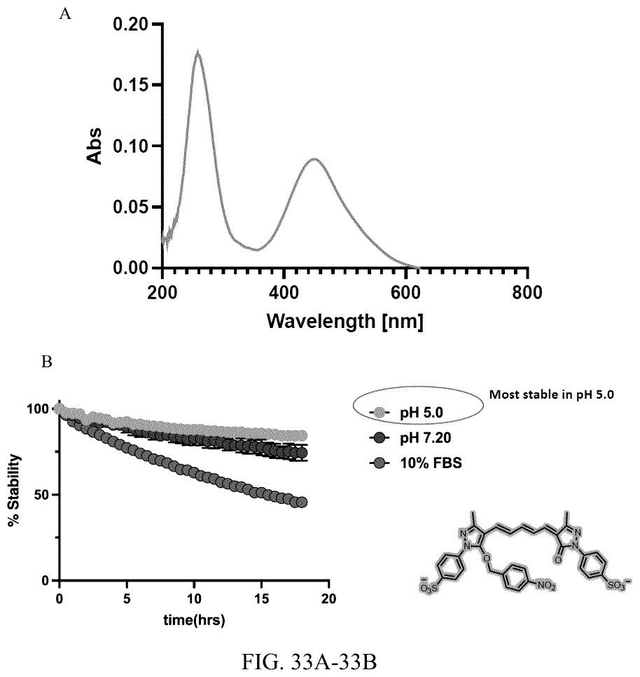

Results: UV-Vis data shoes that there is no absorbance at 600 nm region for full dye, only off dye absorbance at ˜450 nm is detected (FIG. 33A).

### Example 8—Detecting Nitroreductase Activity in BK10083

Method: 1. BK10083 (20 uM in PBS) (Control) 2. BK 10083 (20 uM in PBS)+0.5 mL of 1 Mm NADH (500 uM NADH) 3. BK 10083 (20 uM in PBS)+0.5 mL of 1 Mm NADH (500 uM NADH)+100 μL of 5 uM NTR (500 nM).

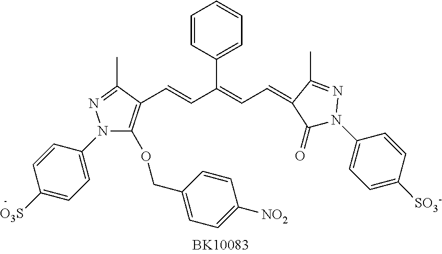

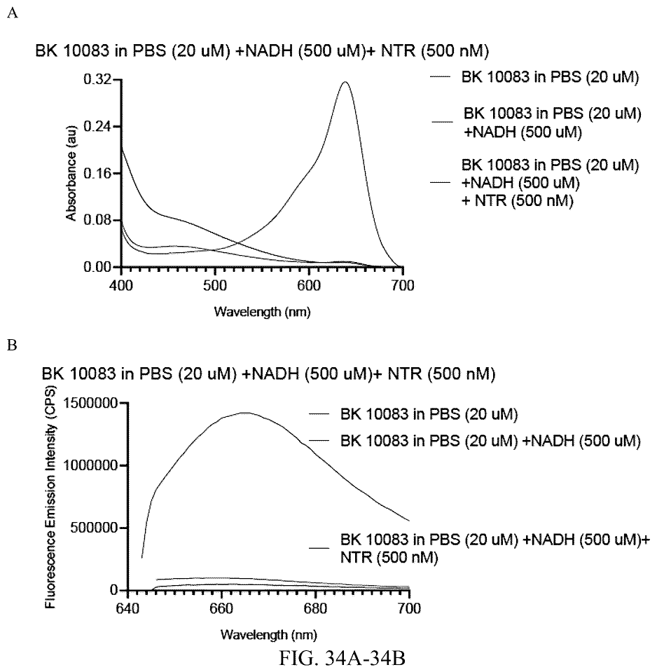

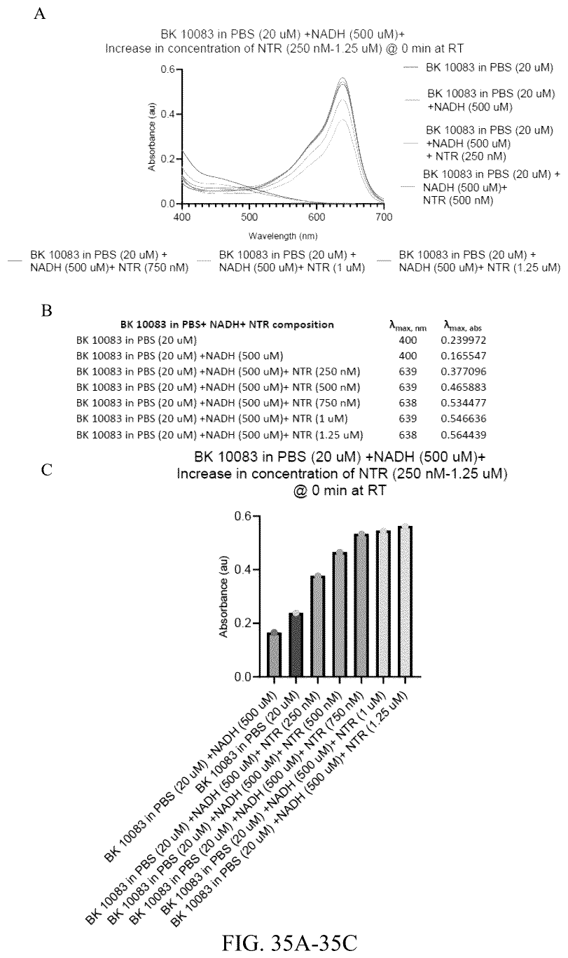

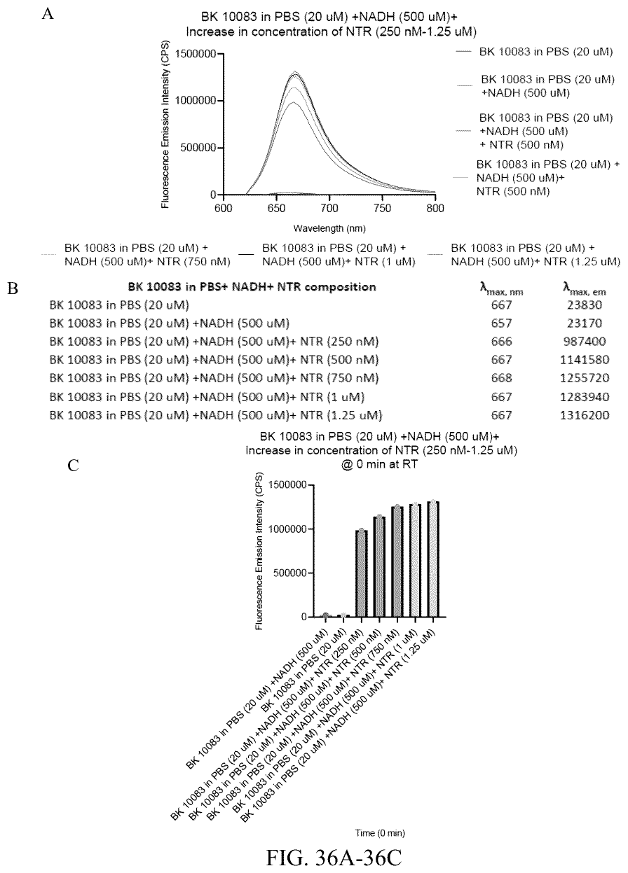

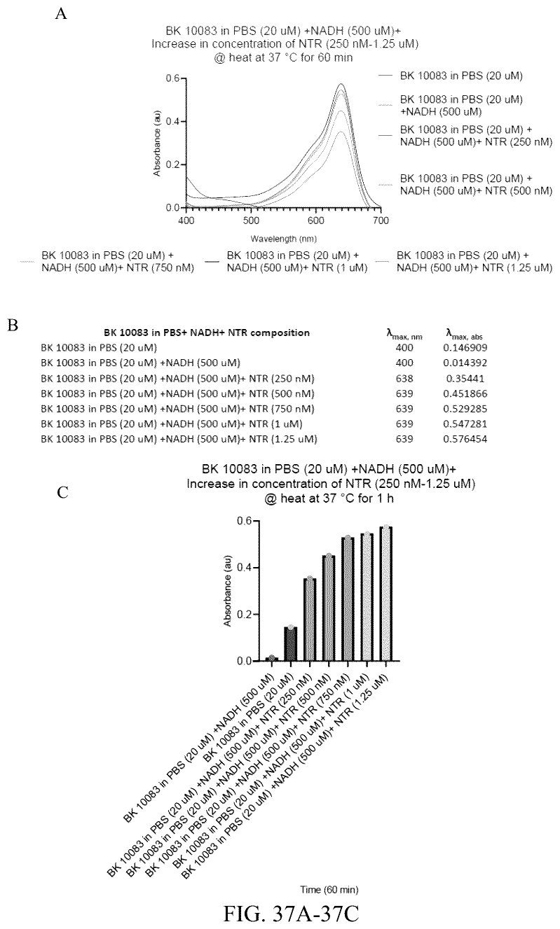

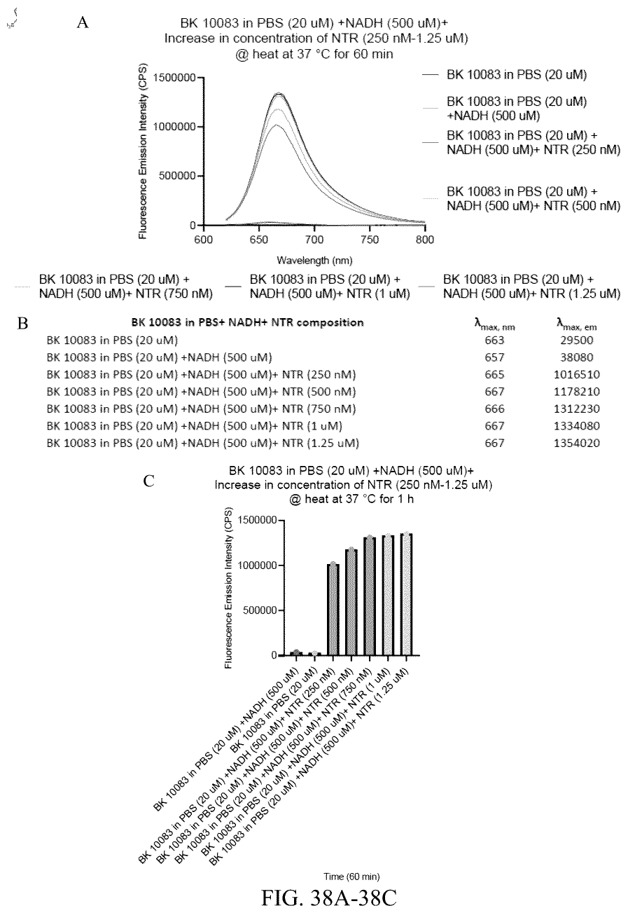

Results: Absorbance and fluorescence emission intensity can be found in FIGS. 34A and 34B. Increase in Concentration of NTR enzyme Vs Absorbance @RT at 0 min is shown in FIG. 35A-35C. Increase in Concentration of NTR enzyme Vs Fluorescence @RT at 0 min is shown in FIG. 36A-36C. Increase in Concentration of NTR enzyme Vs Absorbance @heat at 37° C. for 1 hr can be seen in FIG. 37A-37C. Increase in Concentration of NTR enzyme Vs Fluorescence @heat at 37° C. for 1 hr can be seen in FIG. 38A-38C.

### Example 9—Detecting Nitroreductase Activity in BK10085

Method: 1. BK 10085 (20 uM in PBS) (Control) 2. BK 10085 (20 uM in PBS)+0.5 mL of 1 Mm NADH (500 uM NADH) 3. BK 10085 (20 uM in PBS)+0.5 mL of 1 Mm NADH (500 uM NADH)+100 μL of 5 uM NTR (500 nM).

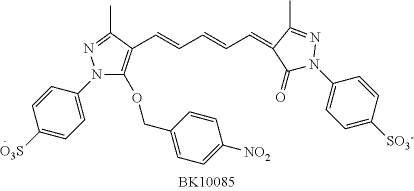

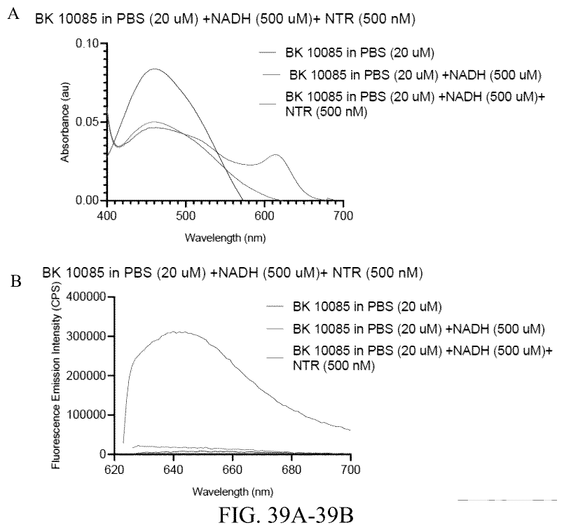

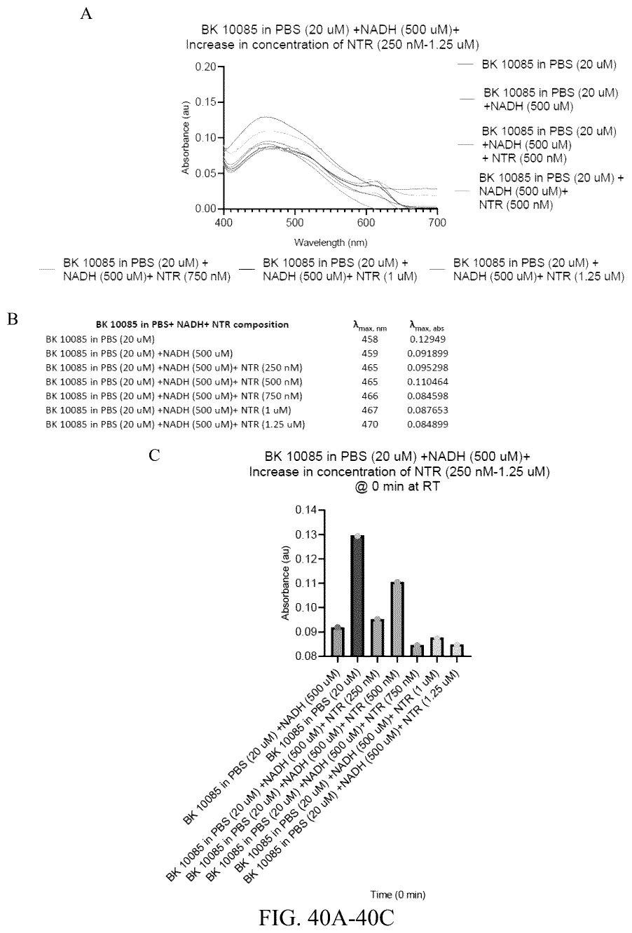

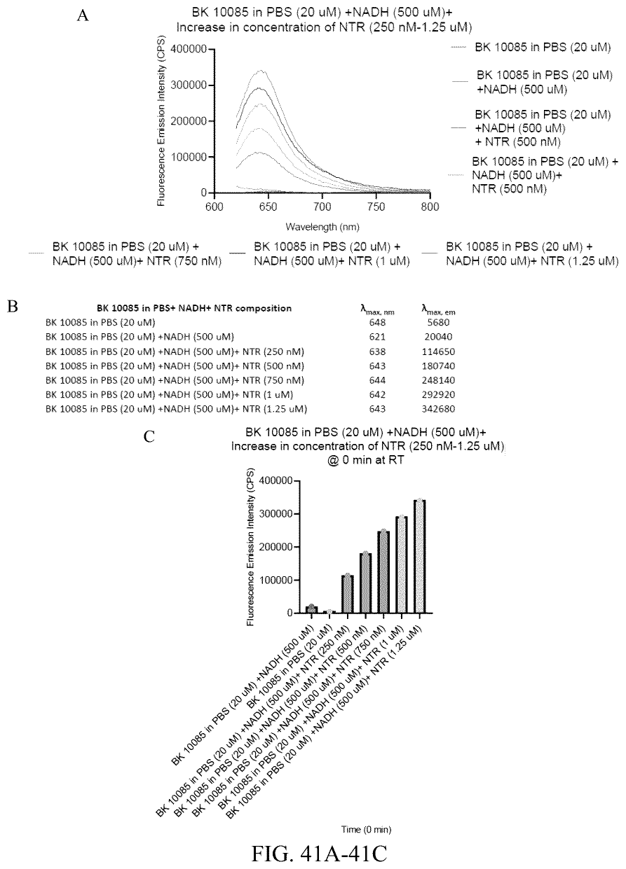

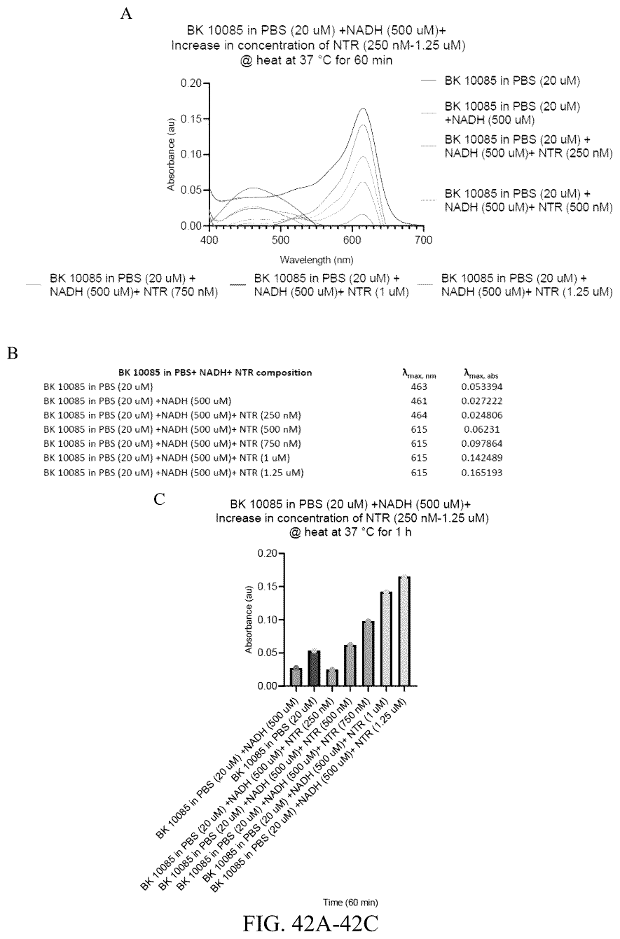

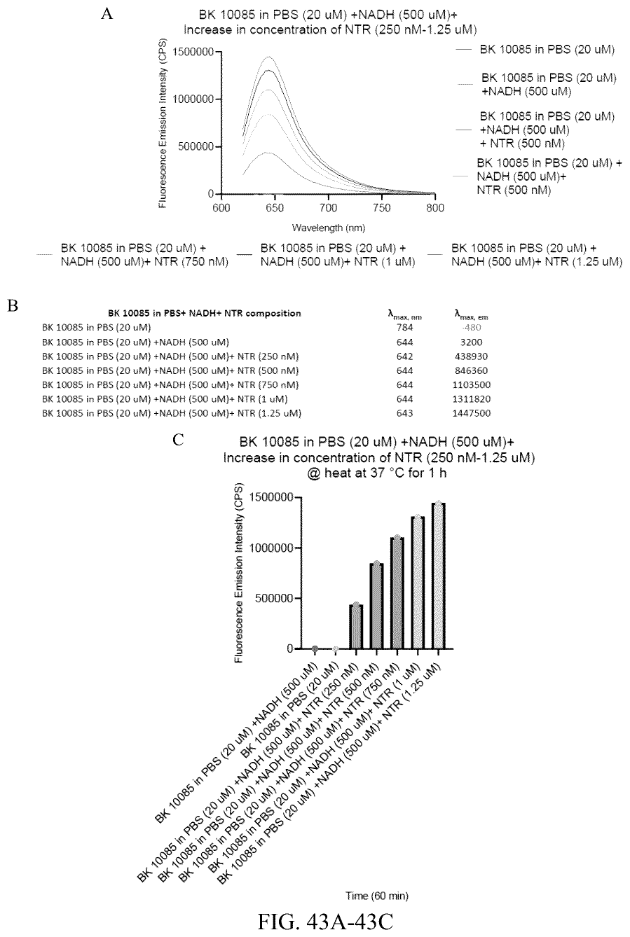

Results: Absorbance and fluorescence emission intensity can be found in FIGS. 39A and 39B. Increase in Concentration of NTR enzyme Vs Absorbance @RT at 0 min can be seen in FIG. 40A-40C. Increase in Concentration of NTR enzyme Vs Fluorescence @RT at 0 min can be seen in FIG. 41A-41C. Increase in Concentration of NTR enzyme Vs Absorbance @heat at 37° C. for 1 hr can be seen in FIG. 42A-42C. Increase in Concentration of NTR enzyme Vs Fluorescence @heat at 37° C. for 1 hr can be seen in FIG. 43A-43C.

### Example 10—Development of Conjugatable SAT-IR742-β

Added compound I and compound II were added to a 20 mL microwave vial and heated it at 70° C. for 12 hours. Purification: The solution was dissolved in isopropanol and precipitated in ether. 70.86% yield.

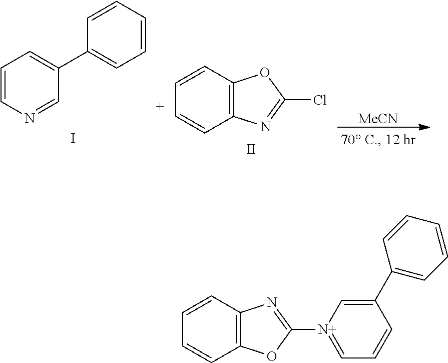

Compound I and compound II were added to a 20 mL microwave vial and heated it at 100° C. for 18 hours. The Solution was that of a pink color. Purification: dissolved in acetone and then precipitated in DCM, vortexed and then decanted final wash with ethyl acetate. % yield: 56.31%.

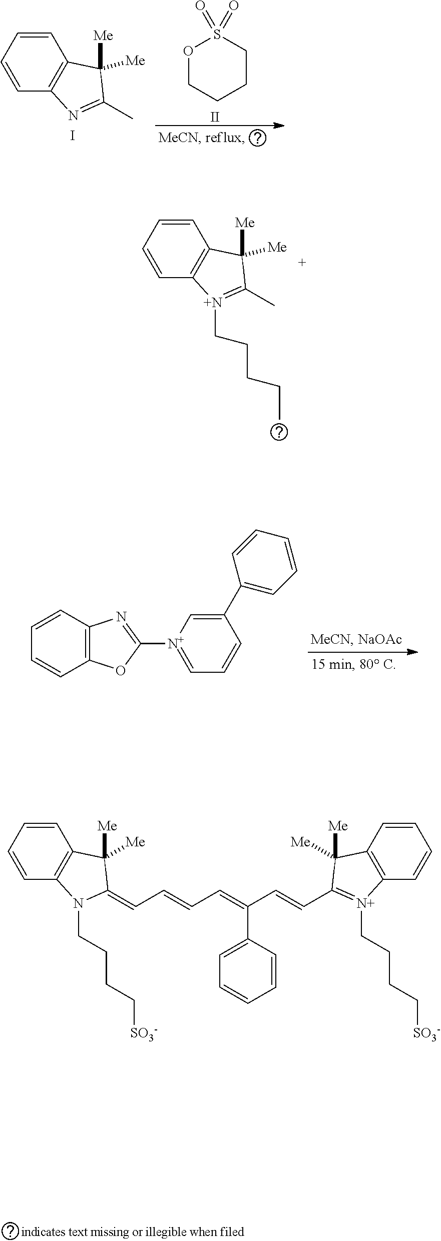

Compounds II and III as well as Acetonitrile (MeCN) and Sodium Acetate (NaOAc) were heated solution at 80° C. for 15 minutes. Reaction products were run on Normal phase ISCO with Acetonitrile and Dichloromethane. Product liquid solution dried in Genevac.

Further Development of Conjugatable SAT-IR742-β. Compounds I and II were added to a 20 mL vial as well as Acetonitrile (MeCN) and heated solution at 70° C. for 18 hours. Followed by precipitation in ether, vortex, centrifuged and dried overnight for a 68% yield.

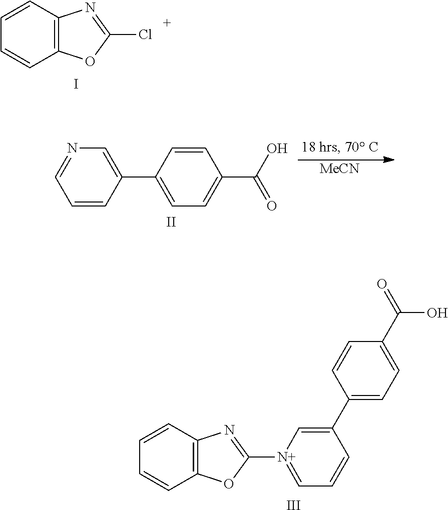

Compounds I and II were added to a 20 mL microwave vial as well as Acetonitrile (MeCN) and Sodium Acetate (NaOAc) heated for 15 minutes at 80° C. Reaction products were run through reverse phase ISCO with a gradient of 0-50% using Acetonitrile and MiliQ Water. Product was dried overnight in the Genevac. 41% yield.

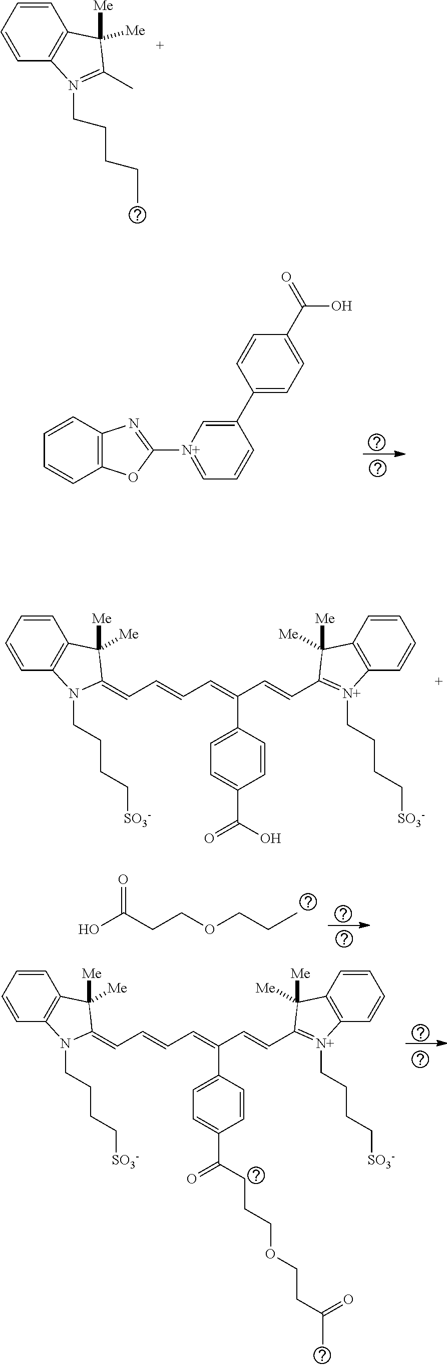

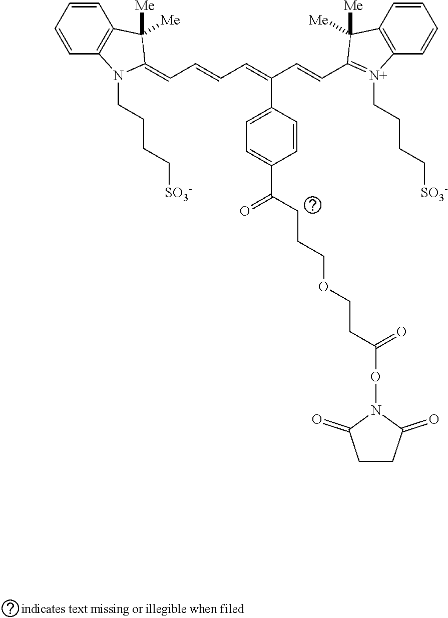

Compound III was added to a 20 mL microwave vial as well as DMF, HATU, and DIPEA stirred for 30 minutes then added compound IV and continued stirring for 12 hours. Reaction products were dissolved in methanol and precipitated in isopropanol. Vortexed and centrifuged then dried overnight in the vacuum.

Compound V was added to a 20 mL microwave vial with DIPEA, DMF, and TSTU stirred solution for 30 minutes at 25 C then added NHS and continued stirring for 2 more hours.

### Example 11—Development of Conjugatable SAT-IR742-β NHS

Compounds I and II were added to a 20 mL vial as well as Acetonitrile (MeCN) and heated solution at 70° C. for 18 hours. Followed by precipitation in ether, vortexed, centrifuged, and dried overnight for a 68% yield.

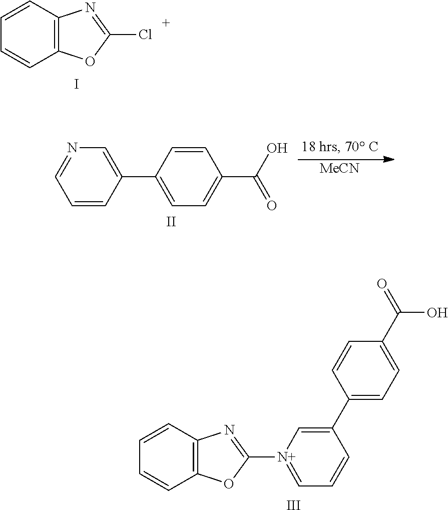

Compounds I and II were added to a 20 mL microwave vial as well as Acetonitrile (MeCN) and Sodium Acetate (NaOAc) heated solution up for 15 minutes at 80° C. The reaction products were ran through a reverse phase ISCO with a gradient of 0-50% using Acetonitrile and MiliQ Water and dried overnight in the Genevac.

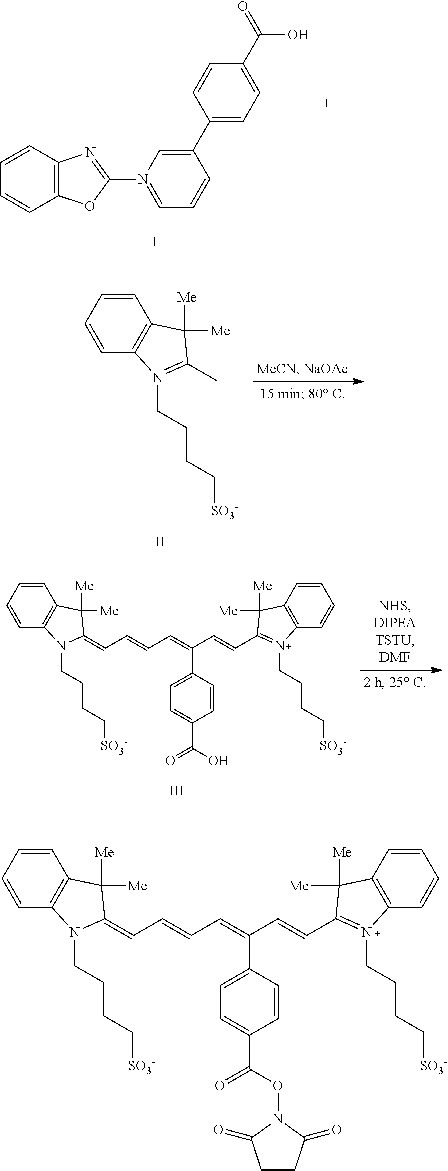

Compound III and compound IV were added to a 20 mL microwave vial as well as TSTU, DMF and DIPEA stirred solution at 25° C. for 30 minutes then added the NHS Ester stirred for 2 hours at 25° C. Reaction product was precipitated in Ether, vortexed, centrifuged, and dried overnight in Genevac.

### Example 12—Development of Conjugatable SAT-IR742-β Maleimide

Compounds I and II were added to a 20 mL vial as well as Acetonitrile (MeCN) and heated solution at 70° C. for 18 hours. Reaction product was precipitated in ether, vortex, centrifuged, and dried overnight with a 68% yield.

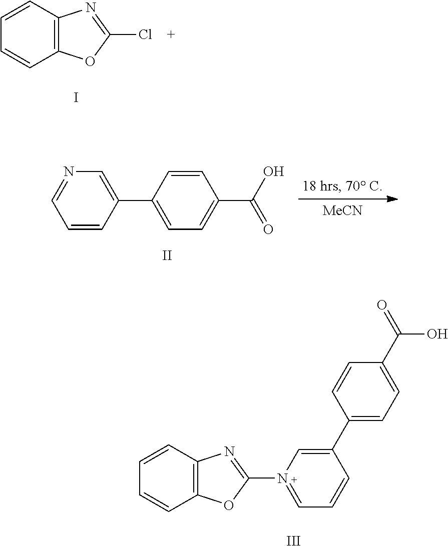

Compounds I and II were added to a 20 mL microwave vial as well as Acetonitrile (MeCN) and Sodium Acetate (NaOAc) heated solution up for 15 minutes at 80° C. Reaction products were ran through reverse phase ISCO with a gradient of 0-50% using Acetonitrile and MiliQ Water and precipitated and dried overnight in the Genevac with a 41% yield.

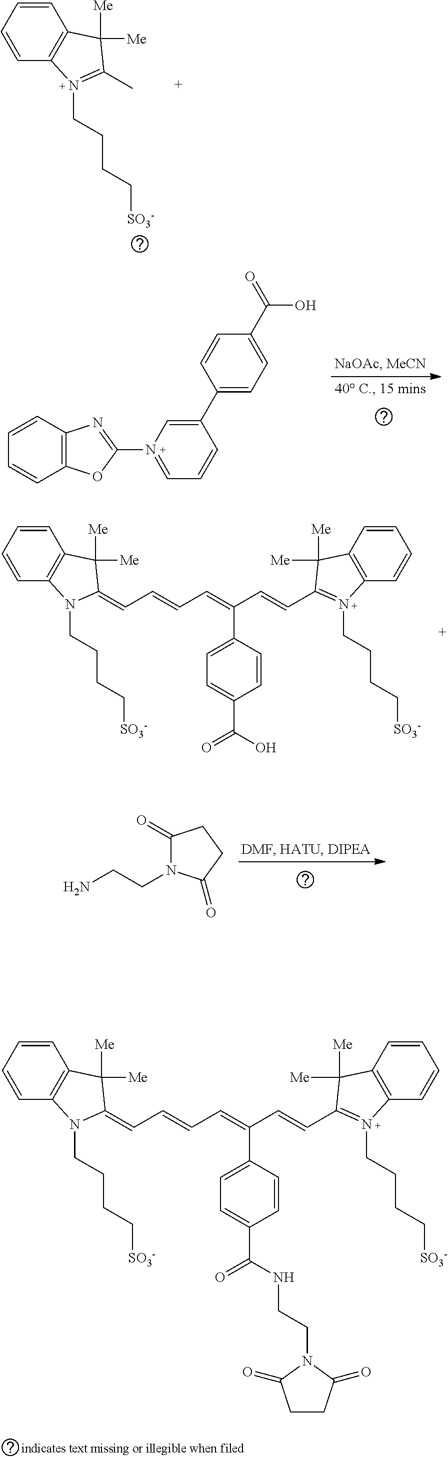

Compound III was added to a 20 mL microwave vial as well as DMF, HATU and DIPEA stirred at 25° C. for 30 minutes then added compound IV then stirred for 2 hours at 25° C. The reaction product was precipitated in ethyl acetate vortexed, centrifuged, and dried it overnight in the vacuum.

### Example 13—Development of Conjugatable SAT-IR742-α

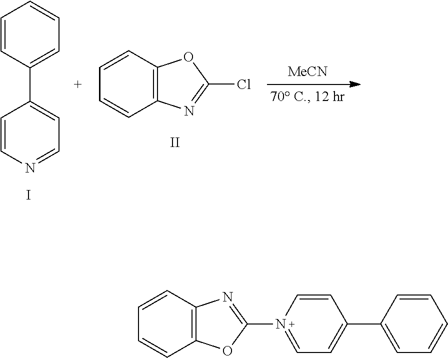

Compound I and compound II were added to a 20 mL microwave vial and heated it at 70° C. For 12 hours. The reaction solution was dissolved in isopropyl and precipitated in ether with an 88% yield.

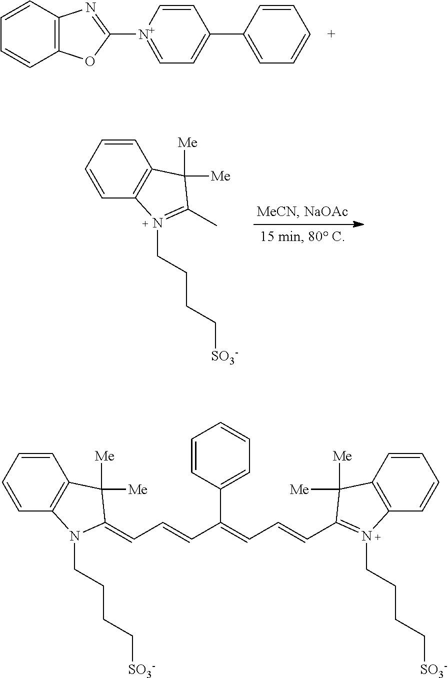

Compound I and compound II were added to a 20 mL microwave vial as well as sodium acetate (NaOAc) and Acetonitrile (MeCN) and heated it at 80° C. for 15 minutes. The solution was blue. The compound was purified using normal phase ISCO at 40% gradient with methanol, and dichloromethane. Product was dried in Genevac overnight with a 71.77% yield.

### Example 14—Development of Conjugatable SAT-IR745-β

Compound I and compound II were added to a 20 mL microwave vial and heated it at 70° C. for 12 hours. The reaction product was dissolved in isopropyl and precipitated in ether with a 70.86% yield.

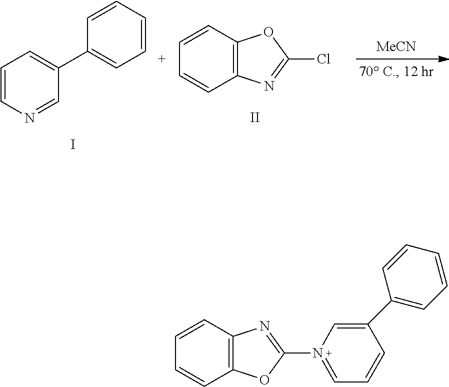

Compound I and compound II were added to a 20 mL microwave vial as well as Acetonitrile (MeCN) and Sodium Iodide and heated at 100° C. for 18 hours. The Solution was red and viscous.

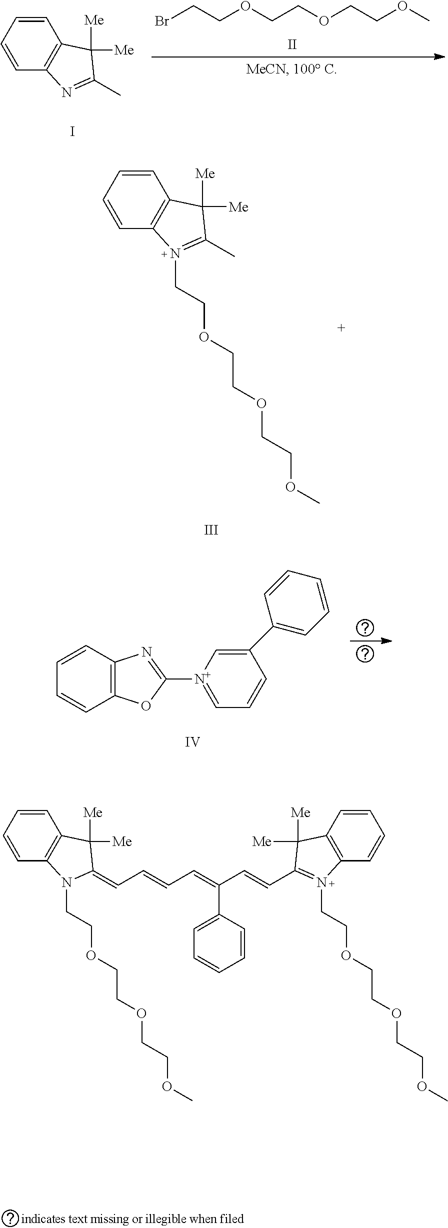

Compound III with compound IV were added to a 20 mL microwave vial as well as Ethanol and heated solution for 15 minutes at 80° C. Solution turned blue. The reaction product was purified by reverse phase ISCO at 5-95% % gradient with Acetonitrile, and MiliQ Water+0.005% Formic Acid. Liquid product was dried in a genevac overnight.

### Example 15—Development of Conjugatable SAT-IR741-β

Compound I and compound II were added to a 20 mL microwave vial and heated it at 70° C. for 12 hours. The solution was dissolved in isopropyl and precipitated in ether resulting in a 70.86% yield.

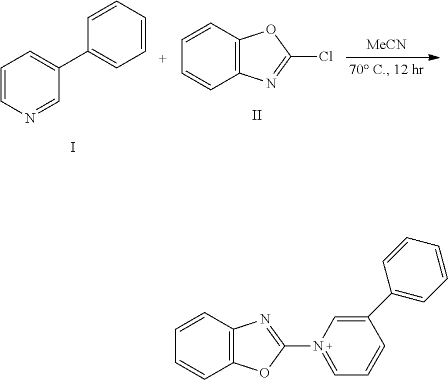

Compound I and compound II were added to a 20 mL microwave vial as well as sodium Iodide (NaI) and Acetonitrile (MeCN) and heated it at 100° C. for 18 hours. Reaction product was dissolved in Acetonitrile and Ether to precipitate, vortexed, centrifuged and let dry overnight.

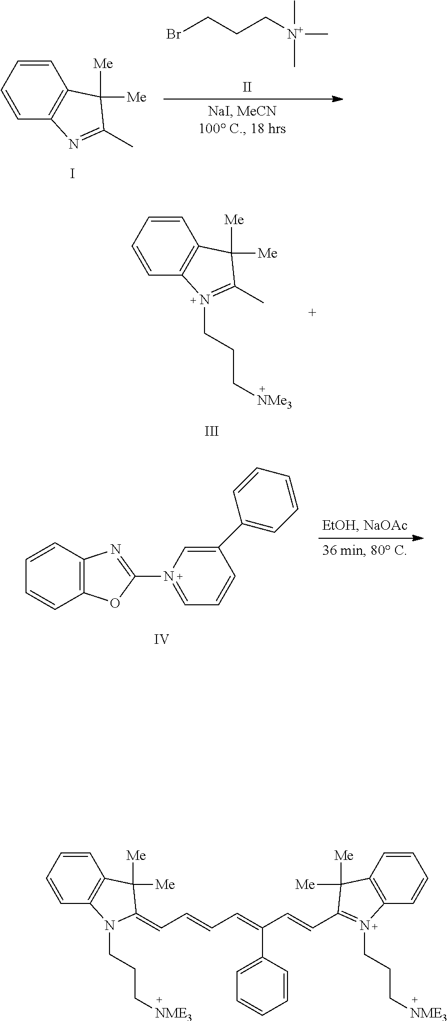

Compound III and compound IV were added to a 20 mL microwave vial as well as sodium Acetate (NaOAc) and Ethanol (EtOH) Heated solution at 80° C. for 35 minutes. The solution turned blue/green. Reaction product was dissolved compound in ethanol then precipitated in isopropanol, vortexed, centrifuged, and dried overnight in Genevac.

### Example 16—Development of Conjugatable SAT-IR746-β

Compound I and compound II were added to a 20 mL microwave vial and heated it at 70° C. for 12 hours. Reaction product was dissolved in isopropyl and precipitated in ether with a 70.86% yield.

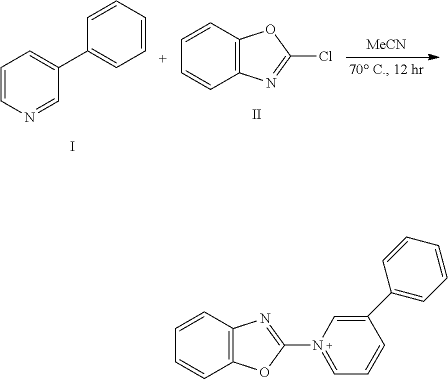

Compound I and compound II were added to a 20 mL microwave vial as well as sodium Iodide and Acetonitrile (MeCN) and heated it at 100° C. for 18 hours. Reaction product was dissolved in Acetonitrile and Ether precipitated, vortexed, centrifuged, and let dry overnight.

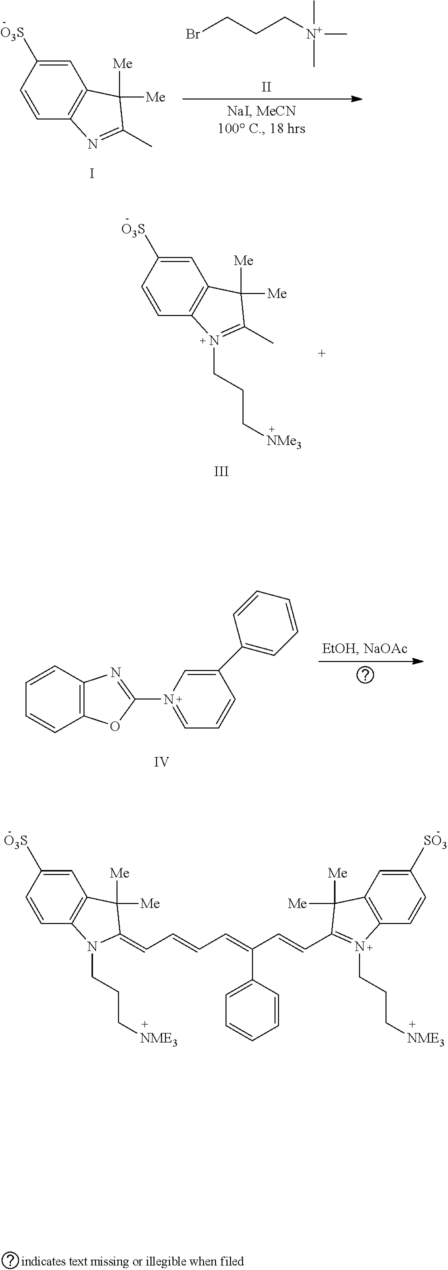

Compound III and compound IV were added to a 20 mL microwave vial as well as sodium Acetate (NaOAc) and Ethanol (EtOH) Heated solution at 80° C. for 35 minutes. The Solution turned blue/green. Solution was dissolved in methanol then precipitated in Ethyl acetate, washed with Isopropanol, vortexed, centrifuged and dried overnight in Genevac.

### Further Development of Conjugatable SAT-IR746-β

Compound I and II were added to a 20 mL vial as well as Acetonitrile (MeCN) and heated solution at 70° C. for 18 hours. Reaction product was precipitated in ether, vortex, centrifuged, and dried overnight with a 68% yield.

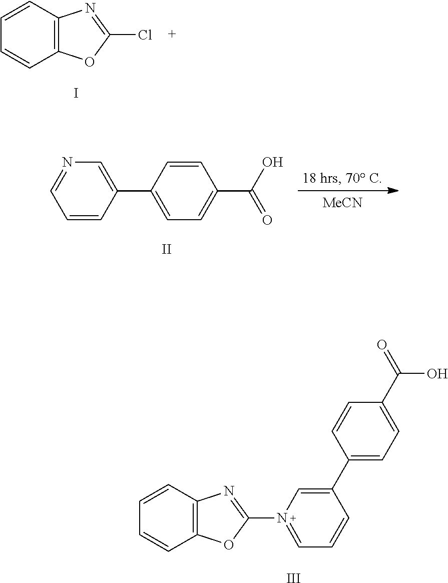

Compound I and compound II added to a 20 mL microwave vial, as well as Acetonitrile (MeCN) and Sodium Acetate (NaOAc), heated solution up for 15 minutes at 80° C. The reaction product was precipitated in ether, vortexed, and centrifuged with a 74% yield.

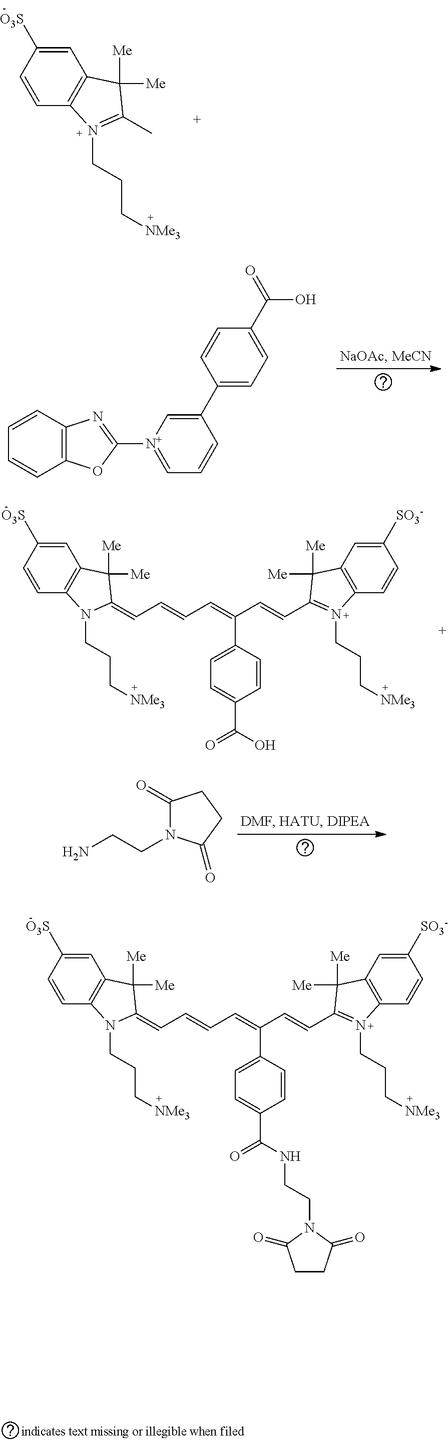

Compound III, DMF, HATU, and DIPEA were added to a 20 mL microwave vial. Heated the solution for 30 minutes then added compound IV and stirred the solution for 2 hours at 25° C. The reaction product was precipitated in ether, vortexed, centrifuged, and dried overnight in Genevac.

### Further Development of Conjugatable SAT-IR746-β

Compound I and compound II were added to a 20 mL vial as well as acetonitrile (MeCN) and heated solution at 70° C. for 18 hours. The reaction product was precipitated in ether, vortexed, centrifuged, and dried overnight with a 68% yield.

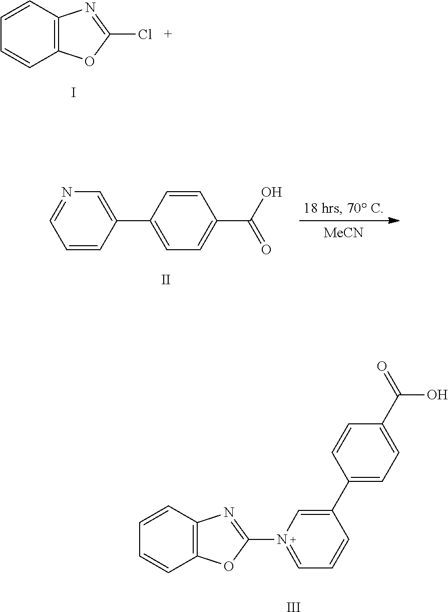

Compound I and compound II was added to a 20 mL microwave vial, as well as acetonitrile (MeCN) and sodium acetate (NaOAc), heated solution up for 15 minutes at 80° C. The reaction product was precipitated in ether, vortexed, and centrifuged with a 74% yield.

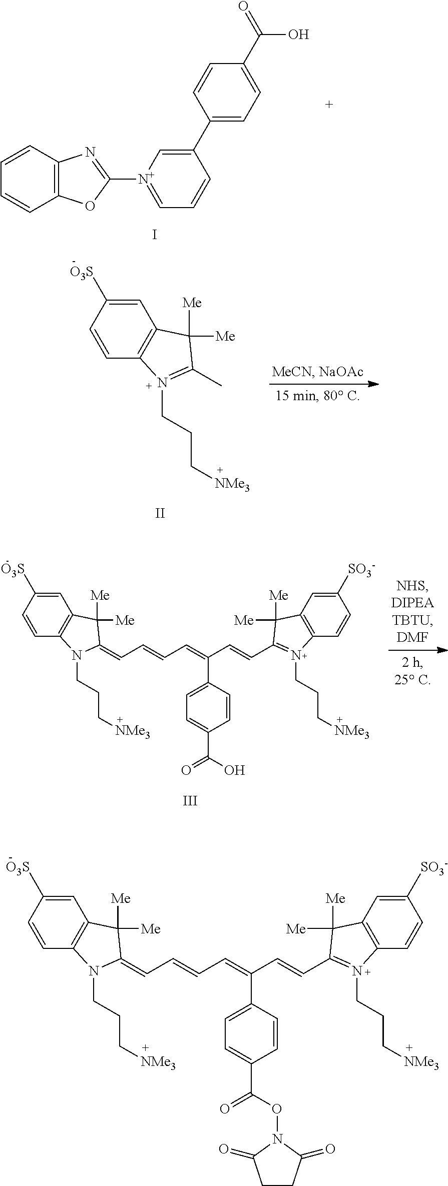

Compound III and compound IV were added to a 20 mL microwave vial as well as TSTU, DMF and DIPEA stirred solution at 25° C. for 30 minutes then added the NHS Ester for 2 hours. Reaction product was precipitated in ether, vortexed, centrifuged, and dried overnight in Genevac.

### Example 17—Thiol Labeling Igg with SAT-IR746-β-L

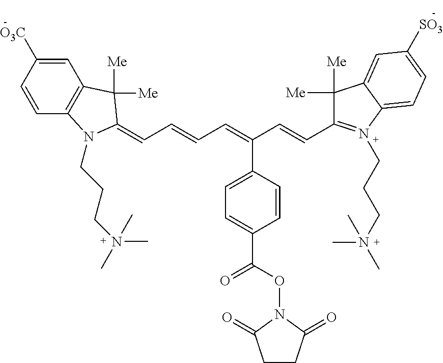

### Example 18—Thiol Labeling SAT-746-β-C

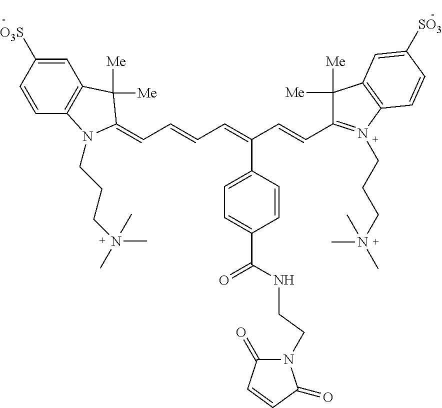

### Example 19—Serum Stability Assay for SAT-746-β

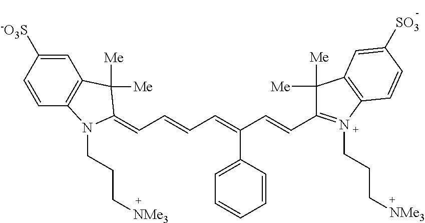

Serum stability was assessed in different solvents (pH 5.0, 10% FBS+Mili Q Water, pH 7.2) by absorbance recorded for every 30 minutes for 18 hrs.

### Example 20—Photostability Assay Comparing SAT-741-β, SAT-746-β, SAT-742-β, and ICG

Photostability assay were performed in PBS pH 7.2 in a 5 mL flask under 700 nM irradiating lamp for 30 minutes taking 0.5 mL every 5 minutes.

### REFERENCES

1. Yi, S. ChemMedChem 2024, 19(15), e202400227.
2. Cheng, P. H. Nat. Rev. Mater. 2021, 6(12), 1095-1113.
3. Usama, S. M. J. Am. Chem. Soc. 2021, 143(15), 5674-5679.
4. Atkinson, K. M. Org. Biomol. Chem. 2021, 19(18), 4100-4106.
5. Yang, Medchemcomm 2018, 9(10), 1754-1760.
6. Lauwerends, Lancet Oncol. 2021, 22(5), E186-E195.
7. Huang, X. Small 2022, 18(34), e2202728.
8. Luo, S. Small 2022, 18(27), e2201298.
9. Yu, Y. ACS Nano 2024, 18(20), 13019-13034.
10. Zhao, X. Acc. Chem. Res. 2024, 57(17), 2582-2593.
11. Usama, S. M. ACS Appl. Bio Mater. 2018.
12. Shrestha, P. J. Am. Chem. Soc. 2025, 147(10), 8280-8288.
13. Du, B. Bioconjug. Chem. 2020, 31(2), 241-247.
14. Olson, M. T. Mol. Cancer Ther. 2020, 19(8), 1670-1681.
15. Zhang, Y. Bioeng. Transl. Med. 2023, 8(4), e10532.
16. Mahalingam, S. M. J Med. Chem. 2018, 61(21), 9637-9646.
17. Tanyi, J. L. J. Clin. Oncol. 2023, 41(2), 276-284.
18. Nishio, N. Mol. Imaging Biol. 2020, 22(1), 156-164.
19. Mieog, J. S. D. Nat. Rev. Clin. Oncol. 2022, 19(1), 9-22.
20. Zhang, R. R. Nat. Rev. Clin. Oncol. 2017, 14(6), 347-364.
21. Stibbe, J. A. Lancet Oncol. 2023, 24(5), 457-467.
22. Warram, J. M. Br. J. Neurosurg. 2015, 29(6), 850-858.
23. Gamage, R. S. Chem. Biomed. Imaging 2024, 2(5), 384-397.
24. Rotermund, F. Chem. Phys. 1997, 220(3), 385-392.
25. Mauerer, M. J. Photochem. Photobiol. B 1998, 47(1), 68-73.
26. Li, B. Angew. Chem. Int. Ed. 2018, 57(25), 7483-7487.
27. Meador, W. E. J. Org. Chem. 2020, 85(6), 4089-4095.
28. Gayton, J. J. Org. Chem. 2019, 84(2), 687-697.
29. Ndaleh, D. J. Org. Chem. 2021, 86(21), 15376-15386.
30. Cosco, E. D. J. Am. Chem. Soc. 2021, 143(18), 6836-6846.
31. Cosco, E. D. Nat. Chem. 2020, 12(12), 1123-1130.
32. Cosco, E. D. Angew. Chem. Int. Ed. 2017, 56(42), 13126-13129.
33. Miranda, D. Theranostics 2019, 9(2), 381-390.
34. Kumari, P. ACS Appl. Bio Mater. 2024, 7(8), 5121-5135.
35. Gadde, S. J. Am. Chem. Soc. 2008, 130(50), 17114-17119.
36. Mordon, S. Microvasc. Res. 1998, 55(2), 146-152.
37. Ogawa, M. Cancer Res. 2009, 69(4), 1268-1272.
38. Zhou, Y. Bioconjug. Chem. 2014, 25(10), 1801-1810.
39. Tadatsu, Y. J. Med. Invest. 2006, 53(1-2), 52-60.
40. Tadatsu, M. Bioorg. Med. Chem. 2003, 11(15), 3289-3294.
41. Choi, H. S. Nat. Biotechnol. 2013, 31(2), 148-153.
42. Choi, H. S. Angew. Chem. Int. Ed. 2011, 50(28), 6258-6263.
43. Usama, S. M. J Am. Chem. Soc. 2023, 145(27), 14647-14659.
44. Li, D. H. Angew. Chem. Int. Ed. 2020, 59(29), 12154-12161.
45. Heist, R. S. J. Clin. Oncol. 2017, 35(24), 2790-2797.
46. Bhattacharyya, S. Medchemcomm 2014, 5(9), 1337-1346.
47. Rosenthal, E. L. Clin. Cancer Res. 2015, 21(16), 3658-3666.
48. Guo, L. JACS Au 2024, 4(8), 3007-3017.
49. Thavornpradit, S. Theranostics 2019, 9(10), 2856-2867.
50. Usama, S. M. Biosens. Bioelectron. 2022, 217, 114610.
51. Henary, M. Dyes Pigm. 2013, 99(3), 1107-1116.
52. Zeng, S. J Med. Chem. 2025, 68(8), 8174-8189.
53. Zhegalova, N. G. Contrast Media Mol. Imaging 2014, 9(5), 355-362.
54. Babity, S. J Org. Chem. 2025, 90(13), 4759-4763.
55. Lin, Y. Bioconjug. Chem. 2002, 13(3), 605-610.
56. Mujumdar, R. B. Bioconjug. Chem. 1993, 4(2), 105-111.
57. Okoh, O. A. J. Sulfur Chem. 2014, 35(1), 42-56.
58. Stackova, L. J. Am. Chem. Soc. 2019, 141(17), 7155-7162.
59. Lakowicz, J. R. Principles of Fluorescence Spectroscopy; Springer US, 1983; pp 187-215.
60. Ma, X. Angew. Chem. Int. Ed. 2023, 62(3), e202216109.
61. Wurthner, F. Angew. Chem. Int. Ed. 2011, 50(15), 3376-3410.
62. Bricks, J. L. Methods Appl. Fluoresc. 2017, 6(1), 012001.
63. Shigemitsu, H. Chem. Commun. 2021, 57(85), 11217-11220.
64. Haque, M. Chem. Commun. 2021, 57(82), 10689-10702.
65. Saphire, E. O. Science 2001, 293(5532), 1155-1159.
66. Harris, L. J. J. Mol. Biol. 1998, 275(5), 861-872.
67. Rijpkema, M. Mol. Imaging 2015, 14, 348-355.
68. Conner, K. P. Drug Metab. Dispos. 2014, 42(11), 1906-1913.
69. Aldrich, M. B. Mol. Imaging Biol. 2011, 13(1), 32-42.
70. Cilliers, C. Mol. Pharm. 2017, 14(5), 1623-1633.
71. Cohen, R. EJNMMI Res. 2011, 1(1), 31.
72. Withrow, K. P. Otolaryngol. Head Neck Surg. 2007, 137(5), 729-734.
73. Dewey, R. S. Chem. Ind. 1969, (45), 1632.
74. Ballou, B. Cancer Detect. Prev. 1998, 22(3), 251-257.
75. Gruber, H. J. Bioconjug. Chem. 2000, 11(5), 696-704.
76. Wang, F. Trends Cell Biol. 2013, 23(4), 175-184.
77. Pu, T. ACS Appl. Mater. Interfaces 2023, 15(27), 32226-32239.
78. Dang, X. Sci. Rep. 2019, 9(1), 3873.
79. Srivastava, I. ACS Nano 2023, 17(9), 8465-8482.
80. Harun, A. ACS Nano 2025, 19(21), 19757-19776.
81. Bendele, N. Adv. Funct. Mater. 2024, 34(38).

1. A method for synthesizing a non-aggregating unsymmetrical heptamethine cyanine fluorophore, comprising:
(a) preparing a pyridinium benzoxazole (PyBox) intermediate by reacting a pyridine derivative substituted at the 3- or 4-position with 2-chlorobenzoxazole in acetonitrile, optionally with sodium iodide; and
(b) synthesizing the non-aggregating unsymmetrical heptamethine cyanine fluorophore by reacting the PyBox intermediate with a sulfonated indolenine and a substituted indolenine in ethanol with sodium acetate, optionally adding triethylamine; and
(c) purifying the product.

2. The method of claim 1, wherein the unsymmetrical heptamethine cyanine is substituted at the β-position of the polymethine chain.

3. The method of claim 2, wherein the unsymmetrical heptamethine cyanine is SAT-IR-746.

4. A non-aggregating unsymmetrical heptamethine cyanine fluorophore produced by the method of claim 1.

5. The non-aggregating unsymmetrical heptamethine cyanine fluorophore of claim 4, further comprising a bioconjugation handle selected from the group consisting of NHS ester, maleimide, carboxylic acid, or thiol-reactive group.

6. A composition comprising the non-aggregating unsymmetrical heptamethine cyanine fluorophore of claim 4 and a biologically compatible carrier.

7. A method for labeling a biomolecule, comprising contacting the biomolecule with the non-aggregating unsymmetrical heptamethine cyanine fluorophore of claim 4 under conditions sufficient to form a labeled biomolecule, wherein the labeling achieves a DOL of 1-5 without aggregation-induced quenching.

8. The method of claim 7, wherein the biomolecule is selected from the group consisting of an antibody, peptide, nucleic acid, or cellular membrane component.

9. A kit for biomedical imaging, comprising the non-aggregating unsymmetrical heptamethine cyanine fluorophore of claim 4 and instructions for making and/or using the fluorophore.
